SEQUENCE LISTING (46 sequences)

SEQ ID NO: 1 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 2 - DNA, 564 bp, synthetic construct
  source 1..564: mol_type = other DNA
atgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactaa

SEQ ID NO: 3 - DNA, 564 bp, synthetic construct
  source 1..564: mol_type = other DNA
atgactaataagtgcctgctgcagatagccctgctcctgtgttttagcaccacggcactgagtatgtcctataatctgctgggctttctgcagaggtcatcaaacttccaatgccaaaagttgctctggcagctgaacggccggctggagtattgtctgaaagatcggatgaatttcgatattcccgaagaaatcaaacaattgcaacaatttcaaaaggaagatgcagccctgacgatttacgaaatgctgcaaaatattttcgcaatctttaggcaggacagcagctccacaggttggaacgaaaccatagtggaaaatctgctcgcgaacgtgtaccaccaaatcaatcacttgaaaaccgtgctcgaggagaagttggaaaaggaagactttacacgtggcaagcttatgtcttccttgcatctcaagaggtactacggccggatactccactatcttaaagctaaagaatattcccattgcgcgtggacaatcgtgcgcgttgagattctgcgcaatttctattttatcaatcgactgacgggctatttgcggaattga

SEQ ID NO: 4 - DNA, 564 bp, synthetic construct
  source 1..564: mol_type = other DNA
atgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggatgctgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctcagaaactaa

SEQ ID NO: 5 - protein, 189 aa, Homo sapiens
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 6 - DNA, 570 bp, synthetic construct
  source 1..570: mol_type = other DNA
atggcctcgccctttgctttactgatggtcctggtggtgctcagctgcaagtcaagctgctctctgggctgtgatctccctgagacccacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcaccgaactctaccagcagctgaatgacttggaagcctgtgtgatgcaggaggagagggtgggagaaactcccctgatgaatgcggactccatcttggctgtgaagaaatacttccgaagaatcactctctatctgacagagaagaaatacagcccttgtgcctgggaggttgtcagagcagaaatcatgagatccctctctttatcaacaaacttgcaagaaagattaaggaggaaggaataa

SEQ ID NO: 7 - DNA, 570 bp, synthetic construct
  source 1..570: mol_type = other DNA
atggcttctcctttcgcccttctcatggtactcgttgttttgtcctgtaaaagctcctgtagccttggatgcgaccttccggaaacacatagtcttgacaatcgaagaacactgatgttgcttgctcagatgtcacgcattagccccagcagttgccttatggatcgccacgatttcgggttcccgcaagaagaattcgacgggaatcaatttcagaaagcgcctgctatttcagtgctgcacgaattgattcaacaaatatttaatctgttcacaaccaaggacagttccgccgcctgggacgaagatcttctggataagttttgtacggagttgtatcaacaattgaacgatctggaagcttgcgtcatgcaagaagaacgcgtaggcgagaccccactcatgaacgcagattctattctggcagttaagaagtattttaggcggataacgctgtacttgactgaaaagaagtatagtccctgcgcttgggaagtggtgcgggccgagataatgcggtctcttagcctctctaccaatctgcaggagaggctgcgccgtaaagagtag

SEQ ID NO: 8 - DNA, 570 bp, synthetic construct
  source 1..570: mol_type = other DNA
atggcctcaccctttgctttactgatggtcctggtggtgctcagctgcaagtcaagctgctctctgggctgtgatctccctgagacccacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcactgaactctaccagcagctgaatgacttggaagcctgtgtgatgcaggaggagagggtgggagaaactcccctgatgaatgctgactccatcttggctgtgaagaaatacttcagaagaatcactctctatctgacagagaagaaatacagcccttgtgcctgggaggttgtcagagcagaaatcatgagatccctctctttatcaacaaacttgcaagaaagattaaggaggaaggaataa

SEQ ID NO: 9 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 10 - DNA, 501 bp, synthetic construct
  source 1..501: mol_type = other DNA
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaa

SEQ ID NO: 11 - DNA, 501 bp, synthetic construct
  source 1..501: mol_type = other DNA
atgaagtacacctcttacatacttgccttccaactgtgtatagtcctgggaagcctggggtgctattgtcaagatccctacgtcaaggaagctgagaatctgaagaagtacttcaacgccggccactccgacgttgccgacaacggcacgctgtttctgggtatcctgaagaactggaaggaagaatctgatcgaaagatcatgcaatctcagatcgtgtctttctatttcaagctcttcaagaacttcaaggacgaccaatcaattcagaaaagcgtggaaacgataaaagaggatatgaacgttaaattctttaactccaacaagaagaagcgcgacgatttcgagaaattgacgaactacagcgttaccgatctcaacgtgcagagaaaggccatccacgagctgattcaggtaatggccgagctctcacctgcggccaagaccggcaaaagaaagcggagccaaatgcttttcaggggcagacgggcttcacaatga

SEQ ID NO: 12 - DNA, 501 bp, synthetic construct
  source 1..501: mol_type = other DNA
atgaaatatacaagttatatcttggcttttcagctctgcatagttttgggttctcttggctgttactgccaggacccatatgtaaaagaagcagaaaaccttaagaaatattttaatgcaggtcattcagatgtagctgataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaaagggatgactttgaaaagctgactaattattcagtaactgacttgaatgtccaaaggaaagcaatacatgaactcatccaagtgatggctgaactgtcaccagcagctaaaacagggaagaggaaaaggagtcagatgctgtttagaggtaggagagcatcccagtaa

SEQ ID NO: 13 - protein, 182 aa, Mus musculus
MNNRWILHAAFLLCFSTTALSINYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQN

SEQ ID NO: 14 - DNA, 546 bp, synthetic construct
  source 1..546: mol_type = other DNA
aacaacaggtggatcctccacgctgcgttcctgctgtgcttctccaccacagccctctccatcaactataagcagctccagctccaagaaaggacgaacattcggaaatgtcaggagctcctggagcagctgaatggaaagatcaacctcacctacagggcggacttcaagatccctatggagatgacggagaagatgcagaagagttacactgcctttgccatccaagagatgctccagaatgtctttcttgtcttcagaaacaatttctccagcactgggtggaatgagactattgttgtacgtctcctggatgaactccaccagcagacagtgtttctgaagacagtactagaggaaaagcaagaggaaagattgacgtgggagatgtcctcaactgctctccacttgaagagctattactggagggtgcaaaggtaccttaaactcatgaagtacaacagctacgcctggatggtggtccgagcagagatcttcaggaactttctcatcattcgaagacttaccagaaacttccaaaactaa

SEQ ID NO: 15 - protein, 186 aa, Canis lupus
  source 1..186: note = subspecies: familiaris
MTSRCILQTTLLLYFSTMALAMSNDLLRSQLSSSSLECQELLLQLNGTTEYCLKDRINFEIPEEIEKSRQFQKEDIILITHEMFQKIFDIFRRNISRTGWNETTVENLLVKLHWQKEHLEIILEDVKEKENFTWDNRTLLHLKKYYLRIVQYLKAKEYSICAWTIVQAEICRNFFFLNILTDYLQN

SEQ ID NO: 16 - DNA, 558 bp, synthetic construct
  source 1..558: mol_type = other DNA
accagtagatgcatcctccaaacaactctcctgttgtatttctccaccatggctcttgccatgagcaacgacttgcttcgatcccagctaagcagcagcagtttggagtgtcaggagctcctattacagttgaatggaaccactgaatattgcctcaaggacaggataaacttcgagatccctgaggaaatcgagaaatcacgccagttccagaaggaggacatcatattgatcacccatgagatgttccagaagatctttgatattttcaggagaaatatctctagaacaggatggaatgagaccactgtcgagaaccttcttgtgaagctccactggcagaaggaacatctggagataatcctggaggacgtcaaagagaaggaaaacttcacctgggacaacaggactcttctgcacctgaagaaatattacttaaggattgtgcagtacctgaaggccaaggagtacagcatctgtgcctggacaatagtccaagcagaaatctgcaggaactttttcttccttaatatacttacagattatctccagaactga

SEQ ID NO: 17 - protein, 19 aa, synthetic construct
ATNFSLLKQAGDVEENPGP

SEQ ID NO: 18 - protein, 18 aa, synthetic construct
EGRGSLLTCGDVEENPGP

SEQ ID NO: 19 - DNA, 122 bp, synthetic construct
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 20 - DNA, 795 bp, synthetic construct
  source 1..795: mol_type = other DNA
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 21 - DNA, 795 bp, synthetic construct
  source 1..795: mol_type = other DNA
atgattgaacaagatggattgcatgcaggttctccagcagcttgggtggagaggctatttggctatgactgggcacaacagacaattggctgctctgatgcagcagtgttcaggctgtcagcccaggggagaccagttctttttgtcaagactgacctgtcaggtgccctgaatgaactgcaagatgaggcagccagactatcctggctggccaccactggtgttccttgtgcagctgtgcttgatgttgtcactgaagcaggaagggactggctgctattgggtgaagtgccagggcaggatctcctgtcatctcaccttgctcctgcagagaaagtatccatcatggctgatgcaatgagaagactgcatacccttgatccagctacctgcccatttgaccaccaagccaaacatagaattgagagagcaagaactagaatggaagcaggtcttgttgatcaggatgatctggatgaagagcatcaggggcttgccccagcagaactgtttgccaggctcaaggccagcatgccagatggtgaggatcttgttgtgacccatggtgatgcctgcttgccaaatatcatggtggaaaatggcagattttctggattcattgactgtggcagactgggtgtggcagacagatatcaggacatagccttggctaccagagatattgctgaagagcttggtggtgaatgggctgacagattccttgtgctttatggtattgcagctcctgattcccagagaattgccttctatagacttcttgatgagttcttctga

SEQ ID NO: 22 - DNA, 584 bp, synthetic construct
  source 1..584: mol_type = other DNA
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 23 - DNA, 933 bp, synthetic construct
  source 1..933: mol_type = other DNA
ttgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 24 - skipped

SEQ ID NO: 25 - DNA, 367 bp, synthetic construct
  source 1..367: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggt

SEQ ID NO: 26 - DNA, 304 bp, synthetic construct
  source 1..304: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 27 - DNA, 246 bp, synthetic construct
  source 1..246: mol_type = other DNA
ttgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 28 - DNA, 278 bp, synthetic construct
  source 1..278: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 29 - DNA, 315 bp, synthetic construct
  source 1..315: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagc

SEQ ID NO: 30 - DNA, 40 bp, synthetic construct
  source 1..40: mol_type = other DNA
ctctgctaaccatgttcatgccttcttctttttcctacag

SEQ ID NO: 31 - DNA, 6671 bp, synthetic construct
  source 1..6671: mol_type = other DNA
cttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatccgaaggggttcaagctagcttcgaattctgcagtcgacggtaccgcgggcccgggatccaccggtctttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgtggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttgaattgtgtgaaatttcaaatattggattaggaaatacaaagttactgaaagtgaggtactaatgtttataaaataaaaactttttcttgccatttgcagatttaacatttttgagtcaatccaagtgccaccatgcaggaggttcatgattgtgtagagtaagacataattttgttgaggtttaactctgaatacttaatgtggtactgaatacttaatgtggtactgagaggcagcctaactgaccacacagcattcatatttcatgttgttattttctctgatcctcattatggctcttcctgcgggtttgagtcttcatttgggtaatttagttattctttgctgcatttgattaaggaaatacgtatatgggaatttggagatttgtgactgacttcactatgtctaatttttttttgagacggagttttgctctgttgcccaggccggagtgcagtggcacagtcttggctcacagcaacctctacctcctgggttcaagcgattctcctgccttagcctcccgagtagctgggattacaggcatgtgccaccacgcctggctaatttttgtatttttagtagagacggggtttcaccatggtggccaggctgggttctggttgtttatgatctttattttttggtgatctaggaaccaaacaacaagaaattgttgtttcccgtgggtaagaggatccgtaagcaaaggcgaggagctattcaccggggtagtacccatcctagtcgagctagacggcgacgtaaacggccacaaattcagcgtatccggcgagggcgagggcgatgccacctacggcaaactaaccctaaaattcatctgcaccaccggcaaactacccgtaccctggcccaccctcgtaaccaccctaacctacggcgtacagtgcttcagccgctaccccgaccacatgaaacagcacgacttcttcaaatccgccatgcccgaaggctacgtccaggagcgcaccatcttcttcaaagacgacggcaactacaaaacccgcgccgaggtcaaattcgagggcgacaccctagtcaaccgcatcgagctaaaaggcatcgacttcaaagaggacggcaacatcctagggcacaaactagagtacaactacaacagccacaacgtctatatcatggccgacaaacagaaaaacggcatcaaagtcaacttcaaaatccgccacaacatcgaggacggcagcgtacagctcgccgaccactaccagcagaacacccccatcggcgacggccccgtactactacccgacaaccactacctaagcacccagtccgccctaagcaaagaccccaacgagaaacgcgatcacatggtcctactagagttcgttaccgccgccgggatcactctcggcatggacgagctatacaaataagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgct

SEQ ID NO: 32 - DNA, 1158 bp, synthetic construct
  source 1..1158: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgtggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttgaattgtgtgaaatttcaaatattggattaggaaatacaaagttactgaaagtgaggtactaatgtttataaaataaaaactttttcttgccatttgcagatttaacatttttgagtcaatccaagtatgcaggaggttcatgattgtgtagagtaagacataattttgttgaggtttaactctgaatacttaatgtggtactgaatacttaatgtggtactgagaggcagcctaactgaccacacagcattcatatttcatgttgttattttctctgatcctcattatggctcttcctgcgggtttgagtcttcatttgggtaatttagttattctttgctgcatttgattaaggaaatacgtatatgggaatttggagatttgtgactgacttcactatgtctaatttttttttgagacggagttttgctctgttgcccaggccggagtgcagtggcacagtcttggctcacagcaacctctacctcctgggttcaagcgattctcctgccttagcctcccgagtagctgggattacaggcatgtgccaccacgcctggctaatttttgtatttttagtagagacggggtttcaccatggtggccaggctgggttctggttgtttatgatctttattttttggtgatctaggaaccaaacaacaagaaattgttgtttcccgtgggtaaga

SEQ ID NO: 33 - DNA, 572 bp, synthetic construct
  source 1..572: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacaggaattgtgtgaaatttcaaatattggattaggaaatacaaagttactgaaagtgaggtactaatgtttataaaataaaaactttttcttgccatttgcagatttaacatttttgagtcaatccaagtgccaccatgcaggaggttcatgattgtgtagagtaagacataattttgttgaggtttaactctgaatacttaatgtggtactgaatacttaatgtggtactgagaggcagcctaactgaccacacagcattcacgcctggctaatttttgtatttttagtagagacggggtttcaccatggtggccaggctgggttctggttgtttatgatctttattttttggtgatctaggaaccaaacaacaagaaattgttgtttcccgtgggtaagaggatcc

SEQ ID NO: 34 - DNA, 861 bp, synthetic construct
  source 1..861: mol_type = other DNA
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 35 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 36 - DNA, 717 bp, synthetic construct
  source 1..717: mol_type = other DNA
gtaagcaaaggcgaggagctattcaccggggtagtacccatcctagtcgagctagacggcgacgtaaacggccacaaattcagcgtatccggcgagggcgagggcgatgccacctacggcaaactaaccctaaaattcatctgcaccaccggcaaactacccgtaccctggcccaccctcgtaaccaccctaacctacggcgtacagtgcttcagccgctaccccgaccacatgaaacagcacgacttcttcaaatccgccatgcccgaaggctacgtccaggagcgcaccatcttcttcaaagacgacggcaactacaaaacccgcgccgaggtcaaattcgagggcgacaccctagtcaaccgcatcgagctaaaaggcatcgacttcaaagaggacggcaacatcctagggcacaaactagagtacaactacaacagccacaacgtctatatcatggccgacaaacagaaaaacggcatcaaagtcaacttcaaaatccgccacaacatcgaggacggcagcgtacagctcgccgaccactaccagcagaacacccccatcggcgacggccccgtactactacccgacaaccactacctaagcacccagtccgccctaagcaaagaccccaacgagaaacgcgatcacatggtcctactagagttcgttaccgccgccgggatcactctcggcatggacgagctatacaaataa

SEQ ID NO: 37 - DNA, 735 bp, synthetic construct
  source 1..735: mol_type = other DNA
atggtgagcaagggcgaggagctgatcaaggagaacatgcacatgaagctgtacatggaaggcaccgtgaacaaccaccacttcaagtgcaccaccgaaggggagggcaagccctacgagggcacccagacccagaggattaaggtggtggagggaggccccctgccgttcgcattcgacatcctggccacctgctttatgtacgggagcaagaccttcatcaaccacacccagggcatccccgatttctttaagcagtccttccctgagggcttcacatgggagagagtcaccacatacgaagacgggggcgtgcttaccgttacccaggacaccagcctccaggacggctgcttgatctacaacgtcaagctcagaggggtgaacttcccatccaacggccctgtgatgcagaagaaaacactcggctgggaggccaccaccgagaccctgtaccccgctgacggcggcctggaaggcagatgcgacatggccctgaagctcgtgggcgggggccacctgcactgcaacctgaagaccacatacagatccaagaaacccgctaagaacctcaagatgcccggcgtctactttgtggaccgcagactggaaagaatcaaggaggccgacaatgagacctacgtcgagcagcacgaggtggctgtggccagatactgcgacctccctagcaaactggggcacaaacttaatggcatggacgagctgtacaagtaa

SEQ ID NO: 38 - DNA, 732 bp, synthetic construct
  source 1..732: mol_type = other DNA
gtcagcaaaggcgaggagctcatcaaagagaacatgcacatgaaactctacatggaaggcaccgtcaacaaccaccacttcaaatgcaccaccgaaggggagggcaaaccctacgagggcacccagacccagagaattaaagtagtagagggaggccccctcccgttcgcattcgacatcctagccacctgctttatgtacgggagcaaaaccttcatcaaccacacccagggcatccccgatttctttaaacagtccttccctgagggcttcacatgggagagagtcaccacatacgaagacgggggcgtcctaaccgttacccaggacaccagcctccaggacggctgcttaatctacaacgtcaaactcagaggggtcaacttcccatccaacggccctgtcatgcagaaaaaaacactcggctgggaggccaccaccgagaccctgtaccccgctgacggcggcctagaaggcagatgcgacatggccctcaaactcgtaggcgggggccacctacactgcaacctaaaaaccacatacagatccaaaaaacccgctaaaaacctaaaaatgcccggcgtctactttgtagaccgcagactagaaagaatcaaagaggccgacaatgagacctacgtagagcagcacgaggtagctgtagccagatactgcgacctccctagcaaactagggcacaaacttaatggcatggacgagctctacaaataa

SEQ ID NO: 39 - DNA, 1653 bp, synthetic construct
  source 1..1653: mol_type = other DNA
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 40 - DNA, 1650 bp, synthetic construct
  source 1..1650: mol_type = other DNA
gaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 41 - DNA, 1650 bp, synthetic construct
  source 1..1650: mol_type = other DNA
gaagatgccaaaaacattaaaaaaggcccagcgccattctacccactcgaagacgggaccgccggcgagcagctacacaaagccatgaaacgctacgccctagtacccggcaccatcgcctttaccgacgcacatatcgaggtagacattacctacgccgagtacttcgagatgagcgttcggctagcagaagctatgaaacgctatgggctcaatacaaaccatcggatcgtagtctgcagcgagaatagcttacagttcttcatgcccgtcttaggtgccctcttcatcggtgttgctgttgccccagctaacgacatctacaacgagcgcgagctactcaacagcatgggcatcagccagcccaccgtcgtattcgtcagcaaaaaagggctccaaaaaatcctcaacgtccaaaaaaaactaccgatcatacaaaaaatcatcatcatggatagcaaaaccgactaccagggcttccaaagcatgtacaccttcgtcacttcccatttaccacccggcttcaacgagtacgacttcgttcccgagagcttcgaccgggacaaaaccatcgccctcatcatgaacagtagtggcagtaccggattacccaaaggcgtagccctaccgcaccgcaccgcttgtgtccgattcagtcatgcccgcgaccccatcttcggcaaccagatcatccccgacaccgctatcctcagcgtagttccatttcaccacggcttcggcatgttcaccactctaggctacttaatctgcggctttcgggtcgtcctcatgtaccgcttcgaggaggagctattcttacgcagcttacaagactataaaattcaatctgccctcctcgttcccacactatttagcttcttcgctaaaagcactctcatcgacaaatacgacctaagcaacttacacgagatcgccagcggcggggcgccgctcagcaaagaggtaggtgaggccgttgccaaacgcttccacctaccaggcatccgccagggctacggcctcacagaaacaaccagcgccattctcatcacccccgaaggggacgacaaacctggcgcagtaggcaaagtcgttcccttcttcgaggctaaagtcgttgacttagacaccggtaaaacactaggtgtcaaccagcgcggcgagctctgcgtccgtggccccatgatcatgagcggctacgttaacaaccccgaggctacaaacgctctcatcgacaaagacggctggctacacagcggcgacatcgcctactgggacgaggacgagcacttcttcatcgttgaccggctaaaaagcctcatcaaatacaaaggctaccaggtagccccagccgaactagagagcatcctcctccaacaccccaacatcttcgacgccggggtcgccggcctacccgacgacgatgccggcgagctacccgccgcagtcgtcgttctagaacacggtaaaaccatgaccgagaaagagatcgttgactatgttgccagccaggttacaaccgccaaaaaactacgcggtggtgttgtcttcgttgacgaggttcctaaaggactaaccggcaagttagacgcccgcaaaatccgcgagattctcattaaagccaaaaaaggcggcaaaatcgccgtctaa

SEQ ID NO: 42 - DNA, 513 bp, synthetic construct
  source 1..513: mol_type = other DNA
gtcttcacactcgaagatttcgttggggactggcgacagacagccggctacaacctagaccaagtccttgaacagggaggtgtctccagtttatttcagaatctcggggtctccgtaactccgatccaaagaattgtcctcagcggtgaaaatgggctcaaaatcgacatccatgtcatcatcccgtatgaaggtctcagcggcgaccaaatgggccagatcgaaaaaatttttaaagtagtctaccctgtagatgatcatcactttaaagtaatcctccactatggcacactagtaatcgacggggttaccccgaacatgatcgactatttcggacggccgtatgaaggcatcgccgtcttcgacggcaaaaaaatcactgtaacagggaccctctggaacggcaacaaaattatcgacgagcgcctcatcaaccccgacggctccctcctcttccgagtaaccatcaacggagtcaccggctggcggctatgcgaacgcattctagcgtaa

SEQ ID NO: 43 - DNA, 843 bp, synthetic construct
  source 1..843: mol_type = other DNA
gtcagcaaaggcgaggagctcttcaccggggtagtccccatcctagtcgagctagacggcgacgtaaacggccacaaattcagcgtctccggcgagggcgagggcgatgccacctacggcaaactcaccctcaaattcatctgcaccaccggcaaactccccgtcccctggcccaccctcgtcaccaccctcacctacggcgtccagtgcttcagccgctaccccgaccacatgaaacagcacgacttcttcaaatccgccatgcccgaaggctacgtccaggagcgcaccatcttcttcaaagacgacggcaactacaaaacccgcgccgaggtcaaattcgagggcgacaccctagtcaaccgcatcgagctcaaaggcatcgacttcaaagaggacggcaacatcctagggcacaaactagagtacaactacaacagccacaacgtctatatcatggccgacaaacagaaaaacggcatcaaagtcaacttcaaaatccgccacaacatcgaggacggcagcgtccagctcgccgaccactaccagcagaacacccccatcggcgacggccccgtcctcctccccgacaaccactacctcagcacccagtccgccctcagcaaagaccccaacgagaaacgcgatcacatggtcctcctagagttcgtcaccgccgccgggatcactctcggcatggacgagctctacaaaaaacttagccatggcttcccgccggaggtagaggagcaggatgatggcacgctccccatgtcttgtgcccaggagagcgggatggaccgtcaccctgcagcctgtgcttctgctagaatcaatgtctag

SEQ ID NO: 44 - DNA, 991 bp, synthetic construct
  source 1..991: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 45 - DNA, 1648 bp, synthetic construct
  source 1..1648: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 46 - DNA, 561 bp, synthetic construct
  source 1..561: mol_type = other DNA
accaacaaatgtctcctccaaatagctctcctcttatgcttctccactacagctctttccatgagctacaacttacttggattcctacaaagaagcagcaattttcagtgtcagaaactcctctggcaattaaatgggagacttgaatactgcctcaaagacagaatgaactttgacatccctgaggagatcaaacagctccagcagttccagaaagaggatgctgcattaaccatctatgagatgctccagaacatctttgctatcttcagacaagattcatctagcactggctggaatgagactatagttgagaacctcctagctaatgtctatcatcagatcaaccatctcaaaacagtcctagaagaaaaactagagaaagaagatttcaccagaggaaaactcatgagcagtctccacctcaaaagatattatgggagaatcctccattacctcaaagccaaagagtacagtcactgtgcctggaccatagtcagagtagaaatcctaagaaacttttacttcatcaacagacttacaggttacctcagaaactaa

(19) United States
(12) Patent Application Publication
(10) Pub. No.: US 2026/0137809 A1
(43) Pub. Date: May 21, 2026

(54) GENE THERAPY VECTORS FOR TREATMENT OF CANCER

(71) Applicant: Siren Biotechnology, Inc., San Francisco, CA (US)

(72) Inventors: Nicole Kristen Paulk, San Francisco, CA (US); Siti Nur Sarah Morris, Vallejo, CA (US); Nathalie Andree Nancy Clement, Gainesville, CA (US)

(21) Appl. No.: 19/132,578

(22) PCT Filed: Nov. 28, 2023
(86) PCT No.: PCT/US2023/081357

§ 371 Date: May 23, 2025

Related U.S. Application Data
(60) Provisional application No. 63/385,172, filed on Nov. 28, 2022.

(51) Int. Cl. A61K 48/00; A61K 9/00; A61K 38/21; A61P 35/00; C12N 15/86

(52) U.S. Cl. CPC A61K 48/005; A61K 9/0085; A61K 38/215; A61K 48/0075; A61P 35/00; C12N 15/86; C12N 2750/14143; C12N 2830/002; C12N 2840/44

(57) ABSTRACT

Described herein are nucleic acids and recombinant viral vectors comprising said nucleic acids, wherein said nucleic acids comprise an interferon that is regulated by alternative splicing.

### CROSS REFERENCE TO RELATED APPLICATIONS

This application claims the benefit of and priority to U.S. Provisional Application No. 63/385,172, filed Nov. 28, 2022, the entire disclosure of which is hereby incorporated by reference in its entirety for all purposes.

### SUMMARY OF THE DISCLOSURE

Described herein, in some embodiments, are nucleic acids comprising a first polynucleotide comprising a minigene 5′ of a second polynucleotide encoding an interferon, wherein the minigene comprises a splice modulator binding site. In some embodiments, the first polynucleotide comprises an in-frame translation stop codon. In some embodiments, the second polynucleotide further comprises a translation stop codon. In some embodiments, the second polynucleotide does not comprise a start codon. In some embodiments, the second polynucleotide does not comprise an open reading frame. In some embodiments, the nucleic acids further comprise a promoter, an enhancer, an intron, a microRNA, a linker, a splicing element, a regulatory element, or a polyA signal. In some embodiments, the nucleic acids further comprise a promoter. In some embodiments, the promoter is selected from the group consisting of: a mini promoter, an inducible promoter, a constitutive promoter, and derivatives thereof. In some embodiments, the promoter is selected from the group consisting of: CMV, CBA, EF1a, CAG, PGK, TRE, U6, UAS, T7, Sp6, lac, araBad, trp, Ptac, p5, p19, p40, Synapsin, CaMKII, GRK1, and derivatives thereof. In some embodiments, the first polynucleotide and second polynucleotide are linked by a cleavable peptide. In some embodiments, the cleavable peptide is a self-cleaving peptide, a drug-sensitive protease, or a substrate for an endogenous endoprotease. In some embodiments, the minigene is regulated by a small molecule splicing modifier. In some embodiments, the small molecule splicing modifier is sudemycin, LMI070 (also known as branaplam), RG7916, RG7800, or a salt derivative thereof (e.g., RG7800-HCl). In some embodiments, the small molecule splicing modifier is selected from the group consisting of:

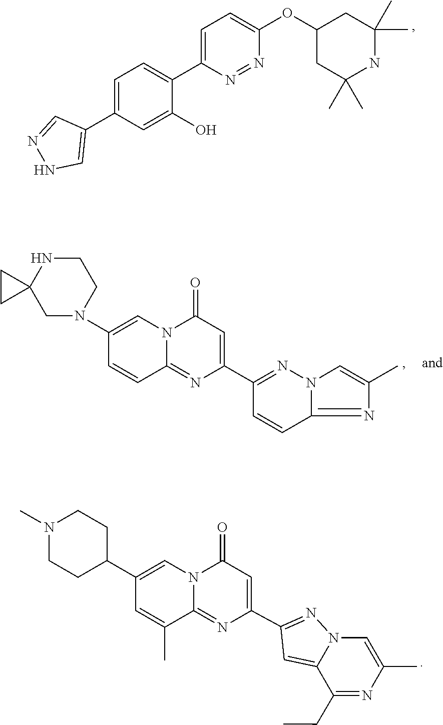

In some embodiments, the minigene comprises exons 6, 7, and 8 of the Survival of Motor Neuron 2 (SMN2) gene (SMN2) and the splice modulator binding site is recognized by LMI070. In some embodiments, the minigene comprises at least about 80% sequence identity to the nucleic acid sequence of SEQ ID NO: 32 or SEQ ID NO: 33. In some embodiments, the minigene is regulated by a disease state in a cell. In some embodiments, the disease state is cancer. In some embodiments, the cancer is glioblastoma. In some embodiments, the minigene is regulated by a cell type or tissue type. In some embodiments, the nucleic acid is provided within a plasmid, a phagemid, a phage derivative, a virus, a bacmid, a bacterial artificial chromosome (BAC), a minicircle, a doggybone, a yeast artificial chromosome (YAC), or a cosmid. In some embodiments, the virus is an alphavirus, a parvovirus, an adenovirus, an adeno-associated virus (AAV), a baculovirus, a Dengue virus, a lentivirus, a herpesvirus, a poxvirus, an anellovirus, a bocavirus, a vaccinia virus, or a retrovirus. In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof. In some embodiments, the herpesvirus (HSV) is HSV type 1 (HSV-1), HSV-2, varicella-zoster virus (VZV), Epstein-Barr virus (EBV), cytomegalovirus (CMV), human herpes virus (HHV) 6 (HHV-6), HHV-7, or HHV-8. In some embodiments, the interferon (IFN) is IFNα, IFNβ, IFNγ, IFNε, IFNκ, IFNω, IFNλ, derivatives thereof, or combinations thereof. In some embodiments, the nucleic acids further comprise a third polynucleotide comprising a minigene 5′ of a fourth polynucleotide encoding an interferon. In some embodiments, the nucleic acids further comprise a fifth polynucleotide comprising a minigene 5′ of a sixth polynucleotide encoding an interferon. In some embodiments, the interferons are the same. In some embodiments, the interferons are different. In some embodiments, the nucleic acids further comprise a backbone polynucleotide. In some embodiments, the backbone polynucleotide comprises an antibiotic resistance gene. In some embodiments, the antibiotic resistance gene encodes an aminoglycoside, a beta-lactam, a macrolide, a tetracycline, or a derivative thereof. In some embodiments, the antibiotic resistance gene encodes a kanamycin, spectinomycin, streptomycin, ampicillin, carbenicillin, bleomycin, erythromycin, polymyxin B, tetracycline, chloramphenicol, neomycin, or zeocin resistance gene, or a derivative thereof. In some embodiments, the antibiotic resistance gene encodes a kanamycin resistance gene. In some embodiments, the kanamycin resistance gene comprises a nucleic acid sequence having at least about 80% sequence identity to the nucleic acid sequence of any one of SEQ ID NOs: 20 or 21. In some embodiments, the antibiotic resistance gene encodes an ampicillin resistance gene. In some embodiments, the ampicillin resistance gene comprises a nucleic acid sequence having at least about 80% sequence identity to the nucleic acid sequence of SEQ ID NO: 34. In some embodiments, the backbone polynucleotide comprises an origin of replication. In some embodiments, the origin of replication is selected from the group consisting of: pMB1, pBR322, ColE1, R6K, p15A, pSC101, ColE2, F1, pUC, and combinations or a derivative thereof. In some embodiments, the viral vector comprises the nucleic acid sequence of SEQ ID NO: 31.

Described herein, in some embodiments, are viral vectors, comprising from 5′ to 3′: (a) a 5′ AAV inverted terminal repeat (ITR); (b) a CAG promoter; (c) a first polynucleotide comprising a minigene, the minigene comprising a splice modulator binding site; (d) a second polynucleotide encoding an interferon; and (e) a 3′ AAV ITR. In some embodiments, the CAG promoter comprises a nucleic acid sequence having at least 80% sequence identity to the nucleic acid sequence of any one of SEQ ID NOs: 22, 23, 44, or 45. In some embodiments, the minigene comprises at least about 80% sequence identity to the nucleic acid sequence of SEQ ID NO: 32 or SEQ ID NO: 33. In some embodiments, the interferon is encoded by a nucleic acid sequence having at least 80% sequence identity to the nucleic acid sequence of any one of SEQ ID NOs: 2-4, 6-8, 10-12, and 46.

Described herein, in some embodiments, are viral vectors, comprising from 5′ to 3′: (a) a 5′ AAV inverted terminal repeat (ITR); (b) a CAG promoter comprising a nucleic acid sequence having at least 80% sequence identity to the nucleic acid sequence of any one of SEQ ID NOs: 22, 23, 44, or 45; (c) a first polynucleotide comprising a minigene, the minigene comprising a splice modulator binding site and at least about 80% sequence identity to the nucleic acid sequence of SEQ ID NO: 32 or SEQ ID NO: 33; (d) a second polynucleotide encoding an interferon, the second polynucleotide comprising at least about 80% sequence identity to the nucleic acid sequence of SEQ ID NO: 4; and (e) a 3′ AAV ITR. In some embodiments, the minigene is regulated by a small molecule splicing modifier. In some embodiments, the small molecule splicing modifier is sudemycin, LMI070, RG7916, RG7800, or a salt derivative thereof. In some embodiments, the salt derivative thereof is RG7800-HCl. In some embodiments, the small molecule splicing modifier is selected from the group consisting of:

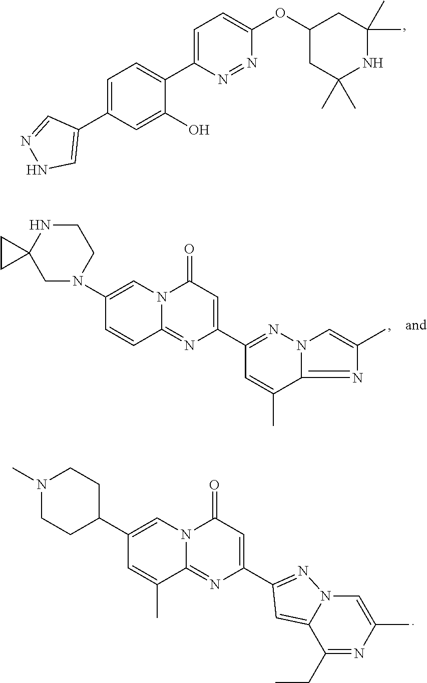

In some embodiments, the viral vector further comprises an antibiotic resistance gene sequence. In some embodiments, the antibiotic resistance gene is a kanamycin resistance gene. In some embodiments, the kanamycin resistance gene comprises a nucleic acid sequence having at least 80% sequence identity to the nucleic acid sequence of any one of SEQ ID NOs: 20 or 21. In some embodiments, the antibiotic resistance gene is an ampicillin resistance gene. In some embodiments, the ampicillin resistance gene comprises a nucleic acid sequence having at least 80% sequence identity to the nucleic acid sequence of any one of SEQ ID NOs: 20 or 21.

Described herein, in some embodiments, are viral vectors comprising a nucleic acid comprising a first polynucleotide comprising a minigene 5′ of a second polynucleotide encoding an interferon, wherein the minigene comprises a splice modulator binding site. In some embodiments, the first polynucleotide comprises an in-frame translation stop codon. In some embodiments, the second polynucleotide further comprises a translation stop codon. In some embodiments, the second polynucleotide does not comprise a start codon. In some embodiments, the second polynucleotide does not comprise an open reading frame. In some embodiments, the viral vectors further comprise a promoter, an enhancer, an intron, a microRNA, a linker, a splicing element, a regulatory element, or a polyA signal. In some embodiments, the viral vectors further comprise a promoter. In some embodiments, the promoter is selected from the group consisting of: a mini promoter, an inducible promoter, a constitutive promoter, and derivatives thereof. In some embodiments, the promoter is selected from the group consisting of: CMV, CBA, EF1a, CAG, PGK, TRE, U6, UAS, T7, Sp6, lac, araBad, trp, Ptac, p5, p19, p40, Synapsin, CaMKII, GRK1, and derivatives thereof. In some embodiments, the first polynucleotide and second polynucleotide are linked by a cleavable peptide. In some embodiments, the cleavable peptide is a self-cleaving peptide, a drug-sensitive protease, or a substrate for an endogenous endoprotease. In some embodiments, the minigene is regulated by a small molecule splicing modifier. In some embodiments, the small molecule splicing modifier is sudemycin, LMI070 (also known as branaplam), RG7916, RG7800, or a salt derivative thereof (e.g., RG7800-HCl). In some embodiments, the small molecule splicing modifier is selected from the group consisting of:

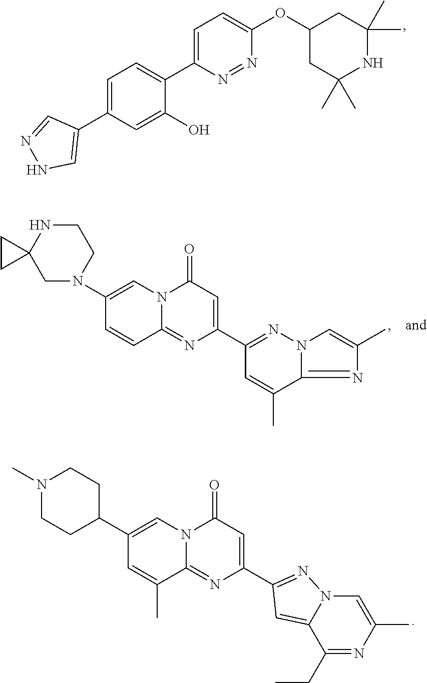

In some embodiments, the minigene comprises exons 6, 7, and 8 of SMN2 and the splice modulator binding site is recognized by LMI070. In some embodiments, the minigene comprises at least about 80% sequence identity to the nucleic acid sequence of SEQ ID NO: 32 or SEQ ID NO: 33. In some embodiments, the minigene is regulated by a disease state in a cell. In some embodiments, the disease state is cancer. In some embodiments, the cancer is glioblastoma. In some embodiments, the minigene is regulated by a cell type or tissue type. In some embodiments, the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus. In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof. In some embodiments, the herpesvirus is HSV-1, HSV-2, VZV, EBV, CMV, HHV-6, HHV-7, or HHV-8. In some embodiments, the interferon (IFN) is IFNα, IFNβ, IFNγ, IFNε, IFNκ, IFNω, IFNλ, derivatives thereof, or combinations thereof. In some embodiments, the viral vectors further comprise a third polynucleotide comprising a minigene 5′ of a fourth polynucleotide encoding an interferon. In some embodiments, the viral vectors further comprise a fifth polynucleotide comprising a minigene 5′ of a sixth polynucleotide encoding an interferon. In some embodiments, the interferons are the same. In some embodiments, the interferons are different. In some embodiments, the viral vectors further comprise a backbone polynucleotide. In some embodiments, the backbone polynucleotide comprises an antibiotic resistance gene. In some embodiments, the antibiotic resistance gene encodes an aminoglycoside, a beta-lactam, a macrolide, a tetracycline, or a derivative thereof. In some embodiments, the antibiotic resistance gene encodes a kanamycin, spectinomycin, streptomycin, ampicillin, carbenicillin, bleomycin, erythromycin, polymyxin B, tetracycline, chloramphenicol, neomycin, or zeocin resistance gene, or a derivative thereof. In some embodiments, the antibiotic resistance gene encodes a kanamycin resistance gene. In some embodiments, the kanamycin resistance gene comprises a nucleic acid sequence having at least about 80% sequence identity to the nucleic acid sequence of any one of SEQ ID NOs: 20 or 21. In some embodiments, the antibiotic resistance gene encodes an ampicillin resistance gene. In some embodiments, the ampicillin resistance gene comprises a nucleic acid sequence having at least about 80% sequence identity to the nucleic acid sequence of SEQ ID NO: 34. In some embodiments, the backbone polynucleotide comprises an origin of replication. In some embodiments, the origin of replication is selected from the group consisting of: pMB1, pBR322, ColE1, R6K, p15A, pSC101, ColE2, F1, pUC, and combinations or a derivative thereof.

Described herein, in some embodiments, are methods of treating cancer in a subject in need thereof comprising: a) administering a nucleic acid comprising a first polynucleotide comprising a minigene 5′ of a second polynucleotide encoding an interferon, wherein the mini gene comprises a splice modulator binding site; and b) administering a small molecule splice modulator for splicing the minigene of step a). In some embodiments, the subject is a human. In some embodiments, the subject is a mouse, rat, equine, sheep, cow, primate, dog, cat, pig, donkey, or rabbit. In some embodiments, the administration is to the central nervous system. In some embodiments, the administration is to a brain. In some embodiments, the administration is to a brain ventricle. In some embodiments, the administration is by convection enhanced delivery (CED). In some embodiments, the administration is by intratumoral injection, intracranial injection, intracerebral injection, intracerebroventricular, or injection into the cerebrospinal fluid (CSF) via the cerebral ventricular system, cisterna magna, or intrathecal space. In some embodiments, the first polynucleotide comprises an in-frame translation stop codon. In some embodiments, the second polynucleotide further comprises a translation stop codon. In some embodiments, the second polynucleotide does not comprise a start codon. In some embodiments, the second polynucleotide does not comprise an open reading frame. In some embodiments, the nucleic acid further comprises a promoter, an enhancer, an intron, a microRNA, a linker, a splicing element, a regulatory element, or a polyA signal. In some embodiments, the nucleic acid further comprises a promoter. In some embodiments, the promoter is selected from the group consisting of: a mini promoter, an inducible promoter, a constitutive promoter, and derivatives thereof. In some embodiments, the promoter is selected from the group consisting of: CMV, CBA, EF1a, CAG, PGK, TRE, U6, UAS, T7, Sp6, lac, araBad, trp, Ptac, p5, p19, p40, Synapsin, CaMKII, GRK1, and derivatives thereof. In some embodiments, the first polynucleotide and the second polynucleotide are linked by a cleavable peptide. In some embodiments, the cleavable peptide is a self-cleaving peptide, a drug-sensitive protease, or a substrate for an endogenous endoprotease. In some embodiments, the minigene is regulated by a small molecule splicing modifier. In some embodiments, the small molecule splicing modifier is sudemycin, LMI070 (also known as branaplam), RG7916, RG7800, or a salt derivative thereof (e.g., RG7800-HCl). In some embodiments, the small molecule splicing modifier is selected from the group consisting of:

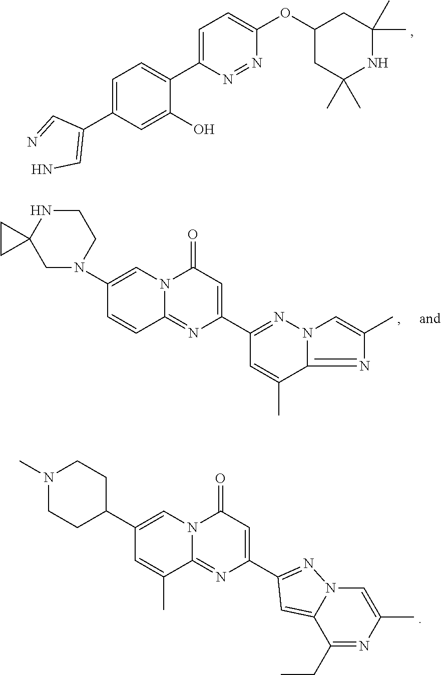

In some embodiments, the minigene comprises exons 6, 7, and 8 of SMN2 and the splice modulator binding site is recognized by LMI070. In some embodiments, the minigene comprises at least about 80% sequence identity to the nucleic acid sequence of SEQ ID NO: 32 or SEQ ID NO: 33. In some embodiments, the minigene is regulated by a disease state in a cell. In some embodiments, the disease state is cancer. In some embodiments, the cancer is glioblastoma. In some embodiments, the minigene is regulated by a cell type or tissue type. In some embodiments, the nucleic acid is provided within a plasmid, a phagemid, a phage derivative, a virus, a bacmid, a bacterial artificial chromosome (BAC), a minicircle, a doggybone, a yeast artificial chromosome (YAC), or a cosmid. In some embodiments, the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus. In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2YF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof. In some embodiments, the herpesvirus is HSV-1, HSV-2, VZV, EBV, CMV, HHV-6, HHV-7, or HHV-8. In some embodiments, the interferon (IFN) is IFNα, IFNβ, IFNγ, IFNε, IFNκ, IFNω, IFNλ, derivatives thereof, or combinations thereof. In some embodiments, the nucleic acid further comprises a third polynucleotide comprising a minigene 5′ of a fourth polynucleotide encoding an interferon. In some embodiments, the nucleic acid further comprises a fifth polynucleotide comprising a minigene 5′ of a sixth polynucleotide encoding an interferon. In some embodiments, the interferons are the same. In some embodiments, the interferons are different. In some embodiments, the nucleic acid further comprises a backbone polynucleotide. In some embodiments, the backbone polynucleotide comprises an antibiotic resistance gene. In some embodiments, the antibiotic resistance gene encodes an aminoglycoside, a beta-lactam, a macrolide, a tetracycline, or a derivative thereof. In some embodiments, the antibiotic resistance gene encodes a kanamycin, spectinomycin, streptomycin, ampicillin, carbenicillin, bleomycin, erythromycin, polymyxin B, tetracycline, chloramphenicol, neomycin, or zeocin resistance gene, or a derivative thereof. In some embodiments, the antibiotic resistance gene encodes a kanamycin resistance gene. In some embodiments, the kanamycin resistance gene comprises a nucleic acid sequence having at least about 80% sequence identity to the nucleic acid sequence of any one of SEQ ID NOs: 20 or 21. In some embodiments, the antibiotic resistance gene encodes an ampicillin resistance gene. In some embodiments, the ampicillin resistance gene comprises a nucleic acid sequence having at least about 80% sequence identity to the nucleic acid sequence of any one of SEQ ID NOs: 20 or 21. In some embodiments, the backbone polynucleotide comprises an origin of replication. In some embodiments, the origin of replication is selected from the group consisting of: pMB1, pBR322, ColE1, R6K, p15A, pSC101, ColE2, F1, pUC, and combinations or a derivative thereof.

### BRIEF DESCRIPTION OF THE DRAWINGS

The disclosure is more completely understood with reference to the following drawings.

FIG. 1 depicts an exemplary inducible AAV expression system plasmid map depicting the various component elements used in the inducible AAV expression systems throughout the specific embodiments.

FIG. 2 depicts an exemplary inducible AAV expression system plasmid map depicting a CAG promoter (variant 3), a splice modulator binding site (“X^{on} splicing element” or “X^{on}”), an eGFP payload (variant 2), and a marker gene that encode resistance to the antibiotic ampicillin (“AmpR”).

FIG. 3 depicts an exemplary inducible AAV expression system plasmid map depicting a CAG promoter (variant 3), X^{on}, a firefly luciferase (fLuc) payload (variant 3), and AmpR.

FIG. 4 depicts an exemplary inducible AAV expression system plasmid map depicting a CAG promoter (variant 3), X^{on}, an eGFP payload (variant 2), and a marker gene that encode resistance to the antibiotic kanamycin (“KanR”).

FIG. 5 depicts an exemplary inducible AAV expression system plasmid map depicting a CAG promoter (variant 3), X^{on}, an fLuc payload (variant 3), and KanR.

FIG. 6 depicts an exemplary inducible AAV expression system plasmid map depicting a CAG promoter (variant 3), X^{on}, a human IFNβ with reduced CpG dinucleotides (“hIFNβ-CpGdel”) payload, and KanR.

FIG. 7 depicts an exemplary inducible AAV expression system plasmid map depicting a CAG promoter (variant 3), X^{on}, an eGFP payload, and AmpR.

FIG. 8 depicts an exemplary inducible AAV expression system plasmid map depicting a short CAG promoter (variant 3), X^{on}, a destabilized eGFP (due to addition of PEST sequence from mouse Odc1 gene; “d2eGFP”) payload, and AmpR.

FIG. 9 depicts an exemplary inducible AAV expression system plasmid map depicting a short CAG promoter (variant 3), X^{on}, an fLuc payload (variant 3), and AmpR.

FIG. 10 depicts an exemplary inducible AAV expression system plasmid map depicting a short CAG promoter (variant 3), X^{on}, a hIFNβ-CpGdel payload, and AmpR.

FIG. 11 depicts an exemplary inducible AAV expression system plasmid map depicting a short CAG promoter (variant 3), X^{on}, a NanoLuc luciferase (nLuc) payload, and AmpR.

FIG. 12 depicts an exemplary inducible AAV expression system plasmid map depicting a CAG promoter (variant 3), X^{on}, an mCardinal (“mCard”) (variant 2) payload, and KanR.

FIG. 13 depicts an exemplary inducible AAV expression system plasmid map depicting a CAG promoter with an intron (variant 4), X^{on}, an mCard payload (variant 2), and KanR.

FIG. 14 depicts an exemplary inducible AAV expression system plasmid map depicting a CAG promoter (variant 3), X^{on}, a hIFNβ-CpGdel payload, and KanR-CpGdel (variant 2).

FIG. 15 depicts an exemplary inducible AAV expression system plasmid map depicting a long CAG promoter with an intron (variant 4), X^{on}, a hIFNβ-CpGdel payload, and KanR-CpGdel (variant 2).

FIG. 16 is a table with the standard human genetic code.

FIG. 17 depicts data from an in vitro validation of inducible AAV-GFP fluorescence.

FIG. 18 depicts data and a set of photomicrographs from an in vitro validation of inducible AAV-eGFP fluorescence (mean±SEM, 2 technical replicates). Photomicrographs from a representative well at multiplicity of infection (MOI) of 1E6 and 2E6 viral genomes (vg)/cell are shown (20× magnification). eGFP positive cells appear in shades of bright grey/white. The graph depicts in vitro data of kinetics of inducible GFP expression by viral dose escalation.

FIG. 19 depicts in vitro data of kinetics of inducible eGFP expression by viral dose escalation. The graph shows the mean fluorescence intensity per well from technical duplicates with readings every 30 minutes for up to 5 hours post-induction with branaplam.

FIGS. 20A-20H depicts in vitro data of inducible AAV-eGFP fluorescence by inducer dose escalation, with FIGS. 20A and 20C depict data with branaplam, FIGS. 20B and 20E depict data with RG7800, FIG. 20D depict data with LMI070, FIG. 20F depict data with RG7800-HCl, FIG. 20G depict data with branaplam and LMI070, and FIG. 20H depict data with RG7800 and RG7800-HCl.

FIGS. 21A-21D depict in vitro data of inducible AAV-eGFP fluorescence by inducer dose escalation, with FIGS. 21A and 21B depict data with branaplam and FIGS. 21C and 21D depict data with RG7800-HCl. The mean and standard deviation of 2 independent experiments are plotted, each performed with technical duplicates. For each experiment, plates were done in duplicate to allow reading for 6 to 7 hours on cells outside of the incubator. The dotted line represents when plate the plate was replaced by its duplicate to maintain cell survival. FIGS. 21A and 21C depict the cell count ratio between eGFP-expressing cells and the total number of cell after induction

FIGS. 22A-22D depict in vitro data of inducible AAV-eGFP fluorescence by inducer dose escalation, with FIGS. 22A and 22B depict data with branaplam and FIGS. 22C and 22D depict data with RG7800-HCl. FIGS. 22A and 22C depict the cell count ratio between eGFP-expressing cells and the total number of cells after induction. The mean and standard deviation of 2 independent experiments are plotted, each performed with technical duplicates.

FIG. 23 depict in vitro inducible AAV-GFP fluorescence by branaplam dose escalation, with the figure depicting the cell count ratio between GFP-expressing cells and the total number of cells after induction.

FIGS. 24A-24B depict in vitro data of inducible AAV-luciferase with branaplam induction, with FIG. 24A depicting data with firefly luciferase and FIG. 24B depicting data with NanoLuc luciferase luminescence in relative light units (RLU). The mean and standard deviation of 2 independent experiments are plotted, each performed with technical duplicates.

FIGS. 25A-25C depict a schematic of experimental design and in vitro data of inducible AAV-fLuc with multiple cycles of branaplam induction. FIG. 25A depicts an experimental schematic of cell transduction with AAV-fLuc. FIG. 25B depicts the relative expression of fLuc from the experiment described in FIG. 25A after the first induction with branaplam with dose escalation. FIG. 25C depicts the relative expression of firefly luciferase from the experiment described in FIG. 25A after the first and second inductions with branaplam, as well as the conditions without branaplam (after first or second induction, respectively) and with branaplam dose escalation. In FIGS. 25B and 25C background luminescence was subtracted out from all readings, and luciferase expression was expressed as relative expression to the signal obtained with the constitutive construct. The mean and standard deviation of 2 independent experiments are plotted, each performed with technical duplicates.

FIGS. 26A-26B depict in vitro data of inducible human IFNβ (hIFNβ) with multiple cycles of branaplam induction, as described in FIG. 25A. FIG. 26A depicts the amount of hIFNβ after the first and second inductions with branaplam, as well as the conditions without branaplam (after first or second induction, respectively) and with branaplam dose escalation. FIG. 26B depicts the relative expression of hIFNβ after the first and second inductions with branaplam, as well as the conditions without branaplam (after first or second induction, respectively) and with branaplam dose escalation. Relative expression was normalized to hIFNβ expression obtained with the constitutive construct. In FIGS. 25A and 25B, the mean and standard deviation of 2 independent experiments are plotted, each performed with technical duplicates.

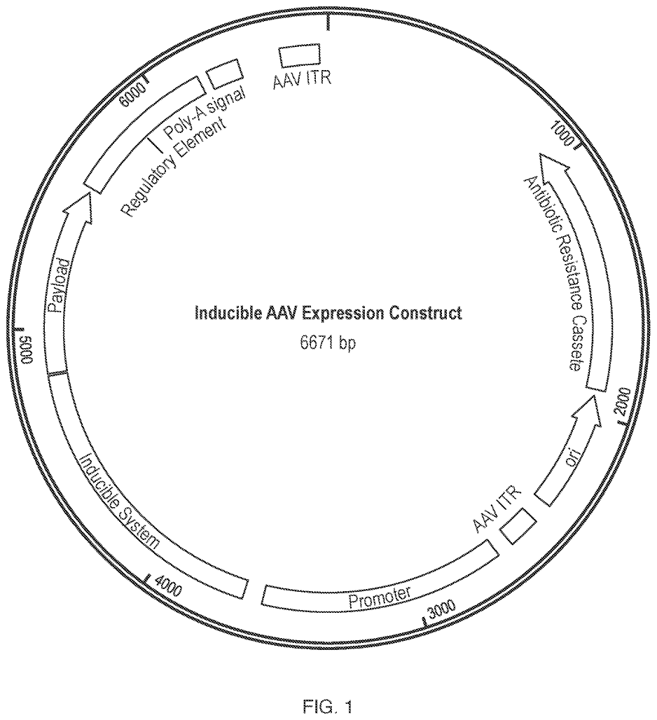

### DETAILED DESCRIPTION

Glioblastoma (GBM) is the most common and deadliest primary malignant brain cancer. Current therapies include surgery, radiation, and chemotherapy. However, complete removal of the tumor has proven difficult, and GBM is often resistant to radiation and chemotherapy. Additionally, chemotherapy is challenging because of the blood brain barrier that prevents effective delivery of reagents.

Additional therapies include small molecule chemotherapies, antibodies, medical devices, personalized CAR-T cell therapies, personalized vaccines, and oncolytic viral therapies. Each has limitations that influence efficacy, safety, or broad applicability for different GBM tumor types.

Type I interferons (IFNs) of the innate immune system play a vital pleiotropic role in helping to treat cancer, acting as endogenous host anti-tumor immuno-oncology agents. Interferon alpha (IFNα1-13) and interferon beta (IFNβ) do this through a variety of direct and indirect actions against tumor cells, including activating the JAK-STAT signaling pathway, recruiting T cells, activating NK cells, and acting as both pro-apoptotic agents and potent inhibitors of angiogenesis. Similarly, type II IFNs (e.g., interferon gamma) have been implicated as important effector molecules of anti-tumor immunity, capable of suppressing tumor growth through various mechanisms. However, clinical trials using IFNs to treat cancer have been hampered by secondary toxicity and the short half-life of IFNs in circulation.

Improved therapies comprising interferons for treating cancer, including glioblastoma, are needed including interferon therapies with improved delivery. Viral vectors are commonly used to deliver therapeutic genes to humans. In particular, adeno-associated viruses (AAV) have been used in recombinant viral vector systems for therapeutic delivery. Provided herein are compositions and methods relating to AAV delivery of regulatable interferon therapies.

### Definitions

Unless defined otherwise, all technical and scientific terms used herein have the same meaning as is commonly understood by one of skill in the art to which the claimed subject matter belongs. Generally, nomenclatures utilized in connection with, and techniques of, immunology, oncology, cell and tissue culture, molecular biology, and protein and oligonucleotide or polynucleotide chemistry and hybridization described herein are those well-known and commonly used in the art. Units of measure not otherwise defined accord with The International System of Units (SI), NIST Special Publication 330, 2019 edition.

As used herein, all numerical values or numerical ranges comprise whole integers within or encompassing such ranges and fractions of the values or the integers within or encompassing ranges unless the context clearly indicates otherwise. Thus, for example, reference to a range of 90-100%, comprises 91%, 92%, 93%, 94%, 95%, 96%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000-fold comprises 1-, 2-, 3-, 4-, 5-, 6-, 7-, 8-, 9-, 10-, 11-, 12-, 13-, 14-, 15-, 16-, 17-, 18-, 19-, or 20-fold, etc., as well as 1.1-, 1.2-, 1.3-, 1.4-, or 1.5-fold, etc., 2.1-, 2.2-, 2.3-, 2.4-, or 2.5-fold, etc., and so forth.

The terminology used herein is for the purpose of describing particular embodiments only and is not intended to be limiting of any embodiment. As used herein, the singular forms “a,” “an,” and “the” are intended to comprise the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” comprises any and all combinations of one or more of the associated listed items.

As used herein, the term “adeno-associated virus vector” or “AAV vector” refers to a vector derived from an AAV serotype, comprising without limitation, AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16. AAV vectors can have one or more of the AAV wild-type genes deleted in whole or part, e.g., the Rep and/or Cap genes, but retain functional flanking inverted terminal repeat (ITR) sequences. Functional ITR sequences promote the rescue, replication, and packaging of the AAV virion. Thus, an AAV vector is defined herein to comprise at least those sequences required in cis for replication and packaging (e.g., functional ITRs) of the virus. ITRs do not need to be the wild-type polynucleotide sequences and, in some embodiments, are altered, e.g., by the insertion, deletion, or substitution of nucleotides, so long as the sequences provide for functional rescue, replication, and packaging.

The terms “adeno-associated virus inverted terminal repeats” or “AAV ITRs” refer to regions flanking each end of the AAV genome which function together in cis as origins of DNA replication and as packaging signals for the virus. AAV ITRs, together with the AAV Rep coding region, can also provide for the efficient excision and integration of a polynucleotide sequence interposed between two flanking ITRs into a mammalian genome. As used herein, an “AAV ITR” does not necessarily comprise the wild-type polynucleotide sequence, because in some embodiments, AAV ITRs herein are altered, e.g., by the insertion, deletion, or substitution of nucleotides. Additionally, the AAV ITRs are derived from any suitable AAV serotype, comprising without limitation AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP.B, AAV-PHP.EB, AAV-2.5, AAV2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, among others. Furthermore, 5′ and 3′ ITRs which flank a selected polynucleotide sequence in an AAV vector need not be identical or derived from the same AAV serotype or isolate, so long as they function as intended, e.g., to allow for the desired therapeutic or genome editing effect. Additionally, AAV ITRs are derived from any of several AAV serotypes, including without limitation, AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP.B, AAV-PHP.EB, AAV-2.5, AAV2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, and AAV-HSC16.

Unless specifically stated or obvious from context, as used herein, the term “about” in reference to a number or range of numbers is understood to mean the stated number and numbers ±10% thereof, or 10% below the lower listed limit and 10% above the higher listed limit for the values listed for a range.

The term “parvovirus” as used herein encompasses the family parvoviridae, comprising but not limited to autonomously replicating parvoviral genera and virus-dependent genera. Autonomous parvoviruses comprise, for example, members of the genus Bocavirus (Bocavirus), the genus Dependovirus (dependovirus), the genus erythro (erythrovirus), the genus mink aleutis virus (Amdovirus), the genus Parvovirus (Parvovirus), the genus Densovirus (Densovirus), the genus repeat virus (iterovirus), the genus cottravirus (containvirus), the genus avarporvovirus, the genus Copiparvovirus, the genus Protoparvovirus (Protoparvovirus), the genus Tetraparvovirus (tetrapivorvirus), the genus ambisense Densovirus (Ambidensovirus), the genus brevicula (brevinnovovirus), the genus hepatodensovirus (hepdensovirus), the genus prawn Densovirus (pendensovirus). Exemplary autonomous parvoviruses comprise, but are not limited to, porcine parvovirus, mouse parvovirus, canine parvovirus, mink enterovirus, bovine parvovirus, chicken parvovirus, feline panleukopenia virus (feline panleukosis virus), feline parvovirus, goose parvovirus, H1 parvovirus, muscovy duck parvovirus, snake parvovirus, and B19 virus.

“Percent identity,” “% identity,” or “sequence identity” refer to the extent to which two sequences (nucleotide or amino acid) have the same residues at the same positions in an alignment. For example, “a nucleotide sequence is X % identical to SEQ ID NO: Y” refers to % identity of the nucleotide sequence to SEQ ID NO: Y and is elaborated as X % of residues in the nucleotide sequence are identical to the corresponding residues of sequence disclosed in SEQ ID NO: Y. A sequence said to be X % identical to a reference sequence may contain more nucleotide or amino acid residues than specified in the reference sequence but must contain a sequence corresponding to the reference sequence. In most cases, the sequence in question will contain a sequence that corresponds to all of the specified reference sequences. Generally, computer programs are employed for such calculations. Exemplary programs that compare and align pairs of sequences, comprise ALIGN, FASTA, gapped BLAST, BLASTP, BLASTN, and GCG.

The term “plasmid” refers to an extrachromosomal element that carries genes that can replicate independently of the chromosomes of the cell. The plasmid can be in the form of a circular double-stranded DNA molecule. Such elements can include autonomously replicating sequences, genome integrating sequences, phage or nucleotide sequences, as well as linear, circular or supercoiled, single- or double-stranded DNA or RNA of any origin. Exemplary plasmids include, but are not limited to, minicircles and doggybone plasmids.

“Polynucleotide” or “nucleic acid” are used interchangeably herein and refer to chains of nucleotides of any length, and comprise DNA and/or RNA. In some embodiments, the nucleotides are deoxyribonucleotides, ribonucleotides, modified nucleotides or bases, and/or their analogs, or any substrate that is incorporated into a chain by DNA or RNA polymerase. A polynucleotide may comprise modified nucleotides, such as methylated nucleotides and their analogs. If present, modification to the nucleotide structure is imparted before or after assembly of the chain. In some embodiments, the sequence of nucleotides is interrupted by non-nucleotide components. In some embodiments, a polynucleotide is further modified after polymerization, such as by conjugation with a labeling component. Other types of modifications comprise, for example, “caps;” substitution of one or more of the naturally occurring nucleotides with an analog; internucleotide modifications such as, for example, those with uncharged linkages (e.g., methyl phosphonates, phosphotriesters, phosphoamidates, and carbamates) and with charged linkages (e.g., phosphorothioates and phosphorodithioates), those containing pendant moieties, such as, for example, proteins (e.g., nucleases, toxins, antibodies, signal peptides, and poly-L-lysine), those with intercalators (e.g., acridine and psoralen), those containing chelators (e.g., metals, radioactive metals, boron, and oxidative metals), those containing alkylators, those with modified linkages (e.g., alpha anomeric nucleic acids); as well as unmodified forms of the polynucleotide(s). In some embodiments, any of the hydroxyl groups ordinarily present in the sugars are replaced, for example, by phosphonate groups, phosphate groups, or protected by standard protecting groups, or activated to prepare additional linkages to additional nucleotides, or conjugated to solid supports. In some embodiments, 5′ and 3′ terminal OH is phosphorylated or substituted with amines or organic capping group moieties of from 1 to 20 carbon atoms. Other hydroxyls may also be derivatized to standard protecting groups. In some embodiments, polynucleotides also contain analogous forms of ribose or deoxyribose sugars, comprising, for example, 2′-O-methyl-, 2′-O-allyl, 2′-fluoro-or 2′-azido-ribose, carbocyclic sugar analogs, alpha-or beta-anomeric sugars, epimeric sugars such as arabinose, xyloses or lyxoses, pyranose sugars, furanose sugars, sedoheptuloses, acyclic analogs and abasic nucleoside analogs such as methyl riboside. In some embodiments, one or more phosphodiester linkages are replaced by alternative linking groups. These alternative linking groups comprise, but are not limited to, embodiments wherein phosphate is replaced by P(O)S(“thioate”), P(S)S (“dithioate”), (O)NRi (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, comprising RNA and DNA.

“Promoter” and “promoter sequence” are used interchangeably and refer to a DNA sequence that controls the expression of a coding sequence or functional RNA. Typically, the coding sequence is located 3′ to the promoter sequence. Promoters can be derived from a native gene, or be composed of different elements derived from different promoters found in nature, or synthetic DNA segments. In some embodiments, different promoters direct the expression of a gene in different tissues or cell types, or at different stages of development, or in response to different environmental or physiological conditions or inducer molecules. Promoters that cause a gene to be expressed in most cell types most of the time are commonly referred to as “constitutive promoters.” Promoters that cause the expression of genes in a particular cell and tissue type are commonly referred to as “cell-specific promoters” or “tissue-specific promoters,” respectively. Promoters that cause the expression of genes at specific stages of development or cell differentiation are commonly referred to as “development-specific promoters” or “cell differentiation-specific promoters.” Promoters that induce and result in the expression of genes after exposing or treating cells with agents, biomolecules, chemicals, ligands, light, etc. that induce the promoters are commonly referred to as “inducible promoters” or “regulatable promoters.” It is further recognized, in some embodiments, that since the exact boundaries of regulatory sequences have not been completely defined in most cases, DNA fragments of different lengths have the same promoter activity.

The phrase “pharmaceutically acceptable” refers to molecular entities and compositions that are physiologically tolerable to a mammal, particularly a human or animal subject.

A “recombinant AAV vector (rAAV vector)” refers to a polynucleotide vector derived from an AAV and comprising one or more heterologous sequences (i.e., nucleic acid sequence not of AAV origin) that are flanked by at least one AAV ITR. In some embodiments, such rAAV vectors are replicated and packaged into viral particles when present in a host cell that has a suitable helper polynucleotide or virus (or that is expressing suitable helper functions) and that expresses AAV Rep and Cap gene products (i.e. AAV Rep and Cap proteins). When a rAAV vector is incorporated into a larger polynucleotide (e.g., in a chromosome or in another vector, such as a plasmid used for cloning or transfection), then the rAAV vector are referred to as a “pro-vector” which are “rescued” by replication and encapsidation in the presence of AAV packaging functions and suitable helper functions.

The term “transgene” refers to a polynucleotide that is introduced into a cell and is capable of being transcribed into RNA by the cell and optionally, translated into a protein and/or expressed under appropriate conditions. The transgene may confer a desired property to a cell into which it was introduced, or otherwise lead to a desired therapeutic or diagnostic outcome.

As used herein, the term “vector” comprises a nucleic acid vector, e.g., a DNA vector, such as a plasmid, an RNA vector, or another suitable replicon (e.g., viral vector). A variety of vectors have been developed for the delivery of polynucleotides encoding exogenous polynucleotides or proteins into a prokaryotic or eukaryotic cell. Examples of such expression vectors are disclosed in, e.g., WO 1994/011026; incorporated herein by reference as it pertains to vectors suitable for the expression of a nucleic acid molecule of interest. Expression vectors suitable for use with the compositions and methods described herein contain a polynucleotide sequence as well as, e.g., additional sequence elements used for the expression of heterologous nucleic acid materials (e.g., a nucleic acid molecule) in a cell. Certain vectors that are used for the expression of the nucleic acid molecules described herein comprise plasmids that contain regulatory sequences, such as promoter and enhancer regions, which direct gene transcription. In some embodiments, the compact bidirectional promoters do not contain an enhancer. Other useful vectors for expression of nucleic acid molecule agents disclosed herein contain polynucleotide sequences that enhance the rate of translation of these polynucleotides or improve the stability or nuclear export of the RNA that results from gene transcription. These sequence elements comprise, e.g., 5′ and 3′ untranslated regions, an internal ribosomal entry site (IRES), and polyadenylation signal (polyA) in order to direct efficient transcription of the gene carried in the expression vector. In some embodiments, the expression vectors suitable for use with the compositions and methods described herein contain a backbone polynucleotide, such as a polynucleotide encoding a marker for selection of cells that contain such a vector. Examples of a suitable marker are genes that encode resistance to antibiotics, such as ampicillin, chloramphenicol, neomycin, zeocin, kanamycin, nourseothricin, aminoglycoside, a beta-lactam, a glycopeptide, a macrolide, a polypeptide, a tetracycline, spectinomycin, streptomycin, carbenicillin, bleomycin, erythromycin, polymyxin B, and chloramphenicol, or a derivative thereof.

As used herein the term “wild-type” refers to the typical form of an organism, strain, gene, nucleic acid, vector, or vector component as it occurs in nature as distinguished from mutant or variant forms.

### Nucleic Acids of the Disclosure

Described herein, in some embodiments, are nucleic acids, comprising a first polynucleotide comprising a minigene 5′ of a second polynucleotide encoding an interferon, wherein the minigene comprises a splice modulator binding site. In some embodiments, the nucleic acids comprise one or more (e.g., 1, 2, 3, 4, 5, 6, 7, 8) exons, introns, pseudoexons, fragments thereof, or combinations thereof.

In some embodiments, the minigene comprises three exons, Exons 1-3, and Exon 2 is skipped in the basal state. When Exon 2 is skipped, the reading frame of Exon 3 is shifted, resulting in the creation of a nonsense mutation in Exon 3. As such, translation of the encoded protein stops in Exon 3, and nothing downstream is translated. Since the coding sequence is located downstream of the minigene, the protein is not expressed in the basal state. Alternatively, translation initiation regulatory sequences are located in Exon 2, and thus when Exon 2 is skipped no translation occurs.

In order to turn on expression of the protein, the inclusion of the skipped exon must be induced. Such induction can occur as a result of the presence of a small molecule splicing modifier. For example, the minigene may comprise Exons 6-8 of the Survival of Motor Neuron 2 (SMN2) gene (SMN2), in which case Exon 7 is skipped in the basal state. However, Exon 7 is included in the presence of certain splicing modifier small molecules (e.g., LMI070). As such, the downstream protein will be expressed in the presence of LMI070, but not expressed in its absence. In some embodiments, the minigene comprises exons 6, 7, and 8 of SMN2 and the splice modulator binding site is recognized by LM1070.

In some embodiments, inclusion of the skipped exon is induced in one cell type, but not another. For example, Exons 8 and 9 of FGFR2 are mutually exclusive, with Exon 9 being only included in mesenchymal tissue (Takeuchi et al., 2010 PLoS One 5.6: e10946). As such, the minigene may comprise Exons 7, 8, 9, and 10 of the FGFR2 gene to allow for expression of the protein only in mesenchymal cells. In this case, a stop codon is engineered into Exon 8 of the minigene to prevent protein expression in non-mesenchymal cells.

Additional examples of cell type-specific alternative splicing events that may be used in an expression control system of the present disclosure include Eps8 Exon 18B (chrl2: 15,792,360-15,792,395) and Eps8 Exon18C (chrl2: 15,787,673-15,787,696), which are specific for auditory hair cells.

In some embodiments, inclusion of the skipped exon is induced by a certain disease state. For example, Huntington's disease results in the generation of transcript isoforms generated by alternative splicing. As such, the minigene may comprise exons from a gene whose splicing is altered in Huntington's disease, i.e., when mutant HTT is expressed, such that an exon that is normally skipped in a healthy cell is included instead (e.g., PCDH1 (5:141869432-141878222). If needed, a stop codon may be engineered into the exon downstream of the alternatively spliced exon to ensure that no protein is produced in non-diseased cells. The result is that the protein will only be expressed when mutant HTT is present. In this example, the target gene may encode an inhibitory RNA that knocks down the expression of mutant HTT, thus creating an autoregulatory feedback loop: the presence of mutant HTT will induce expression of an inhibitory RNA that targets mutant HTT, thereby reducing mutant HTT levels to a level that causes the splicing of the minigene to return to the non-diseased state, thereby turning off the expression of the inhibitory RNA and allowing for expression of mutant HTT, which will reach a level that induces expression of the inhibitory RNA, and so on.

In another example, the minigene comprises three exons, Exons 1-3, and Exon 2 is included in the basal state. Inclusion of Exon 2 can either result in a downstream frameshift such that translation stops in Exon 3; or Exon 2 can be engineered to include a stop codon. As such, when Exon 2 is included, i.e., in the basal state, the protein is not expressed.

In order to turn on expression of the protein, skipping of Exon 2 must be induced. Such can occur as a result of the presence of a small molecule splicing modifier. For example, the minigene may comprise Exons 3, 4, 10, 11, and 12 of the MDM2 gene (Singh et al, 2009 Experimental cell research 315.19:3419-3432), which are all included in the basal state. However, Exons 4, 10, and 11 are skipped in the presence of certain splicing modifier small molecules (e.g., sudemycin) (Shi et al., 2015 Pharmacology research & perspectives 3.4: e00158). As such, the downstream transactivator will be expressed in the presence of sudemycin, but not in its absence. In this case, a stop codon may be engineered into Exon 4 of the minigene to ensure that no protein is produced in the absence of sudemycin.

In some embodiments, the first polynucleotide comprises an in-frame translation stop codon. In some embodiments, alternative splicing of the first polynucleotide removes the in-frame translation stop codon. In some embodiments, alternative splicing of the first polynucleotide modifies the transcript thereby deleting or nullifying the stop codon, introducing an initiation or start codon, restoring the open reading frame, or providing a missing portion of the protein. In some embodiments, alternative splicing of the first polynucleotide allows for transcription and translation of the second polynucleotide.

In some embodiments, the second polynucleotide further comprises a translation stop codon. In some embodiments, the second polynucleotide does not comprise a start codon. In some embodiments, the second polynucleotide does not comprise an open reading frame.

In some embodiments, the first polynucleotide and second polynucleotide are linked by a cleavable peptide. In some embodiments, the cleavable peptide is a self-cleaving peptide, a drug-sensitive protease, or a substrate for an endogenous endoprotease.

In some embodiments, the minigene comprises the nucleic acid sequence of SEQ ID NO: 32 or SEQ ID NO: 33. In some embodiments, the minigene comprises a nucleic acid sequence having at least 70% (e.g., 75%, 80%, 90%, 95%, 96%, 97%, 98%, or 99%) sequence identity to the nucleic acid sequence of SEQ ID NO: 32 or SEQ ID NO: 33. In some embodiments, the minigene comprises a nucleic acid sequence having at least 75% sequence identity to the nucleic acid sequence of SEQ ID NO: 32 or SEQ ID NO: 33. In some embodiments, the minigene comprises a nucleic acid sequence having at least 80% sequence identity to the nucleic acid sequence of SEQ ID NO: 32 or SEQ ID NO: 33. In some embodiments, the minigene comprises a nucleic acid sequence having at least 90% sequence identity to the nucleic acid sequence of SEQ ID NO: 32 or SEQ ID NO: 33. In some embodiments, the minigene comprises a nucleic acid sequence having at least 95% sequence identity to the nucleic acid sequence of SEQ ID NO: 32 or SEQ ID NO: 33. In some embodiments, the minigene comprises a nucleic acid sequence having at least 96% sequence identity to the nucleic acid sequence of SEQ ID NO: 32 or SEQ ID NO: 33. In some embodiments, the minigene comprises a nucleic acid sequence having at least 97% sequence identity to the nucleic acid sequence of SEQ ID NO: 32 or SEQ ID NO: 33. In some embodiments, the minigene comprises a nucleic acid sequence having at least 98% sequence identity to the nucleic acid sequence of SEQ ID NO: 32 or SEQ ID NO: 33. In some embodiments, the minigene comprises a nucleic acid sequence having at least 99% sequence identity to the nucleic acid sequence of SEQ ID NO: 32 or SEQ ID NO: 33.

In some embodiments, the minigene is regulated by a small molecule splicing modifier. In some embodiments, the small molecule splicing modifier is sudemycin (FR901464, pladienolide B), LMI070, RG7916, or RG7800 and derivatives thereof (e.g., a salt derivative thereof e.g., RG7800-HCl).

In some embodiments, the small molecule splicing modifier is LMI070, also known as branaplam, (CAS No.: 1562338-42-4) having the following structure:

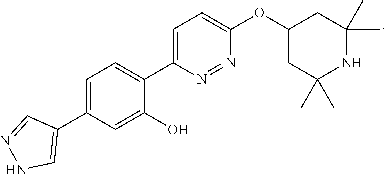 [SMILES: CC1(C)CC(Oc2ccc(-c3ccc(-c4cn[nH]c4)cc3O)nn2)CC(C)(C)N1]

In some embodiments, the small molecule splicing modifier is an analogue of LMI070. Exemplary analogues of LMI070 include, but are not limited to, 6-(naphthalen-2-yl)-N-(2,2,6,6-tetramethylpiperidin-4-yl)pyridazin-3-amine; 6-(benzo[b]thio-phen-2-yl)-N-methyl-N-(2,2,6,6-tetra-methylpiperidin-4-yl)pyridazin-3-amine; 2-(6-(2,2,6,6-tetramethylpiperidin-4-ylamino)-pyridazin-3-yl)phenol; 2-(6-(methyl-(2,2,6,6-tetra-methylpiperidin-4-yl)amino)pyridazin-3-yl)benzo[b]-thiophene-5-carbonitrile; 6-(quinolin-3-yl)-N-(2,2,6,6-tetramethyl-piperidin-4-yl)pyridazin-3-amine; 3-(benzo[b]-thiophen-2-yl)-6-(2,2,6,6-tetra-methylpiperidin-4-yloxy)pyridazine; 2-(6-(methyl-(2,2,6,6-tetra-methylpiperidin-4-yl)amino)-pyridazin-3-yl)phenol; 6-(6-(methyl-(2,2,6,6-tetra-methylpiperidin-4-yl)amino)-pyridazin-3-yl)naphthalen-2-ol; 6-(benzo[b]-thiophen-2-yl)-N-(2,2,6,6-tetra-methylpiperidin-4-yl)pyridazin-3-amine; 7-(6-((2,2,6,6-tetramethylpiperidin-4-yl)oxy)pyridazin-3-yl)isoquinoline; 6-(6-((2,2,6,6-tetramethylpiperidin-4-yl)oxy)pyridazin-3-yl)isoquinoline; N-methyl-6-(quinolin-7-yl)-N-(2,2,6,6-tetramethyl-piperidin-4-yl)pyridazin-3-amine; N-methyl-6-(quinolin-6-yl)-N-(2,2,6,6-tetramethylpiperidin-4-yl)pyridazin-3-amine; 6-(isoquinolin-7-yl)-N-methyl-N-(2,2,6,6-tetramethylpiperidin-4-yl)pyridazin-3-amine; 6-(isoquinolin-6-yl)-N-methyl-N-(2,2,6,6-tetramethylpiperidin-4-yl)pyridazin-3-amine; 6-(imidazo[1,2-a]pyridin-6-yl-pyridazin-3-yl)-methyl-(2,2,6,6-tetramethyl-piperidin-4-yl)-amine; methyl-[6-(6-phenyl-pyridin-3-yl)-pyridazin-3-yl]-(2,2,6,6-tetramethyl-piperidin-4-yl)-amine; methyl-[6-(6-pyrrol-1-yl-pyridin-3-yl)-pyridazin-3-yl]-(2,2,6,6-tetramethyl-piperidin-4-yl)-amine; methyl-[6-(6-pyrazol-1-yl-pyridin-3-yl)-pyridazin-3-yl]-(2,2,6,6-tetramethyl-piperidin-4-yl)-amine; methyl-(6-quinoxalin-2-yl-pyridazin-3-yl)-(2,2,6,6-tetramethyl-piperidin-4-yl)-amine; methyl-(6-quinolin-3-yl-pyridazin-3-yl)-(2,2,6,6-tetramethyl-piperidin-4-yl)-amine; N-methyl-6-(phthalazin-6-yl)-N-(2,2,6,6-tetramethylpiperidin-4-yl)pyridazin-3-amine; 6-(benzo[c][1,2,5]oxa-diazol-5-yl)-N-(2,2,6,6-tetramethyl-piperidin-4-yl)pyridazin-3-amine; 6-(benzo[d]thiazol-5-yl)-N-(2,2,6,6-tetramethyl-piperidin-4-yl)pyridazin-3-amine; 6-(2-methylbenzo-[d]oxazol-6-yl)-N-(2,2,6,6-tetramethyl-piperidin-4-yl)pyridazin-3-amine; 3-(6-(methyl(2,2,6,6-tetramethylpiperidin-4-yl)amino)pyridazin-3-yl)naphthalen-2-ol; 5-chloro-2-(6-(methyl(1,2,2,6,6-pentamethylpiperidin-4-yl)amino)pyridazin-3-yl)phenol; 3-(6-(2,2,6,6-tetramethylpiperidin-4-yl)amino)pyridazin-3-yl)naphthalen-2-ol; 5-chloro-2-(6-(1,2,2,6,6-pentamethylpiperidin-4-yl)amino)pyridazin-3-yl)phenol; 4-hydroxy-3-(6-(methyl(2,2,6,6-tetramethylpiperidin-4-yl)amino)pyridazin-3-yl)benzonitrile; 3-[6-(2,2,6,6-tetramethyl-piperidin-4-yloxy)-pyridazin-3-yl]-naphthalen-2-ol; 2-{6-[methyl-(2,2,6,6-tetramethyl-piperidin-4-yl)-amino]-pyridazin-3-yl}-4-trifluoromethyl-phenol; 2-fluoro-6-{6-[methyl-(2,2,6,6-tetramethyl-piperidin-4-yl)-amino]-pyridazin-3-yl}-phenol; 3,5-dimethoxy-2-{6-[methyl-(2,2,6,6-tetramethyl-piperidin-4-yl)-amino]-pyridazin-3-yl}-phenol; 4,5-dimethoxy-2-{6-[methyl-(2,2,6,6-tetramethyl-piperidin-4-yl)-amino]-pyridazin-3-yl}-phenol; 5-methoxy-2-{6-[methyl-(2,2,6,6-tetramethyl-piperidin-4-yl)-amino]pyridazin-3-yl}-phenol; 4,5-difluoro-2-{6-[methyl-(2,2,6,6-tetramethyl-piperidin-4-yl)-amino]-pyridazin-3-yl}-phenol; 5-fluoro-2-{6-[methyl-(2,2,6,6-tetramethyl-piperidin-4-yl)-amino]-pyridazin-3-yl}-phenol; 3-hydroxy-4-(6-(methyl(2,2,6,6-tetramethylpiperidin-4-yl)amino)pyridazin-3-yl)benzonitrile; 1-allyl-6-(6-(methyl(2,2,6,6-tetramethylpiperidin-4-yl)amino)pyridazin-3-yl)naphthalen-2-ol; 6-(benzo[b]thiophen-2-yl)-N-(1,2,2,6,6-pentamethylpiperidin-4-yl)pyridazin-3-amine; N-allyl-3-hydroxy-4-(6-(methyl(2,2,6,6-tetramethylpiperidin-4-yl)amino)pyridazin-3-yl)benzamide; 2-(6-(methyl(2,2,6,6-tetramethylpiperidin-4-yl)amino)pyridazin-3-yl)-5-(1H-pyrazol-1-yl)phenol; 5-(5-methyl-oxazol-2-yl)-2-{6-[methyl-(2,2,6,6-tetramethyl-piperidin-4-yl)-amino]pyridazin-3-yl}-phenol; 5-(4-hydroxymethyl)-1H-pyrazole-1-yl)-2-(6-(methyl(2,2,6,6-tetramethylpiperidin-4-yl)amino)pyridazin-3-yl)phenol; 5-(1H-imidazole-1-yl)-2-(6-(methyl(2,2,6,6-tetramethyl-piperidin-4-yl)amino)pyridazin-3-yl)phenol; 5-(4-amino-1H-pyrazole-1-yl)-2-(6-(methyl(2,2,6,6-tetramethylpiperidin-4-yl)amino)pyridazin-3-yl)phenol; 5-(4-amino-1H-pyrazol-1-yl)-2-(6-(methyl(2,2,6,6-tetramethylpiperidin-4-yl)amino)pyridazin-3-yl)phenol; 5-(3-amino-pyrazol-1-yl)-2-{6-[methyl-(2,2,6,6-tetramethyl-piperidin-4-yl)-amino]pyridazin-3-yl}-phenol; 2-(6-(methyl(2,2,6,6-tetramethylpiperidin-4-yl)amino)pyridazin-3-yl)-5-(1-(2-morpholino-ethyl)-1H-pyrazol-4-yl)phenol; 2-(6-(methyl (2,2,6,6-tetramethylpiperidin-4-yl)amino)pyridazin-3-yl)-5-(1-methyl-1H-pyrazol-4-yl)phenol; 5-(5-amino-1H-pyrazol-1-yl)-2-(6-(methyl-(2,2,6,6-tetramethyl-piperidin-4-yl)amino)pyridazin-3-yl)phenol; 2-(6-(methyl(2,2,6,6-tetramethylpiperidin-4-yl)amino)pyridazin-3-yl)-4-(1H-pyrazol-1-yl)phenol; 2-{6-[(2-hydroxy-ethyl)-(2,2,6,6-tetramethyl-piperidin-4-yl)-amino]pyridazin-3-yl}-5-pyrazol-1-yl-phenol; 2-(6-(piperidin-4-yloxy)pyridazin-3-yl)-5-(1H-pyrazol-1-yl)phenol; 2-(6-(((2S,4R,6R)-2,6-dimethylpiperidin-4-yl)oxy)pyridazin-3-yl)-5-(1H-pyrazol-1-yl)phenol; 2-(6-((-2,6-di methyl piperidin-4-yl)oxy)pyridazin-3-yl)-5-(1H-pyrazol-1-yl)phenol; 2-(6-((-2,6-dimethyl piperidin-4-yl)oxy)pyridazin-3-yl)-5-(1H-pyrazol-1-yl)phenol; 5-(1H-pyrazol-1-yl)-2-(6-(pyrrolidin-3-yloxy)pyridazin-3-yl)phenol; 2-(6-((-2-methylpiperidin-4-yl)oxy)pyridazin-3-yl)-5-(1H-pyrazol-1-yl)phenol; (S)-5-(1H-Pyrazol-1-yl)-2-(6-(pyrrolidin-3-ylmethoxy)pyridazin-3-yl)phenol; (R)-5-(1H-pyrazol-1-yl)-2-(6-(pyrrolidin-3-ylmethoxy)pyridazin-3-yl)phenol; 2-(6-((3-fluoropiperidin-4-yl)oxy)pyridazin-3-yl)-5-(1H-pyrazol-1-yl)-phenol; 2-[6-(1,2,2,6,6-pentamethyl-piperidin-4-yloxy)-pyridazin-3-yl]-5-pyrazol-1-yl-phenol; 5-pyrazol-1-y 1-2-[6-(2,2,6,6-tetramethyl-piperidin-4-yloxy)-pyridazin-3-yl]-phenol; 5-(1H-Pyrazol-4-yl)-2-(6-((2,2,6,6-tetramethylpiperidin-4-yl)oxy)pyridazin-3-yl)phenol; 2-(6-piperazin-1-yl-pyridazin-3-yl)-5-pyrazol-1-yl-phenol; 3-[6-(azetidin-3-ylamino)-pyridazin-3-yl]-naphthalen-2-ol; 2-[6-(azetidin-3-ylamino)-pyridazin-3-yl]-5-pyrazol-1-yl-phenol; 2-[6-(3,5-dimethyl-piperazin-1-yl)-pyridazin-3-yl]-5-pyrazol-1-yl-phenol; 2-[6-(7-methyl-2,7-diaza-spiro[4.4]non-2-yl)-pyridazin-3-yl]-5-pyrazol-1-yl-phenol; 2-(6-[1,4]diazepan-1-yl-pyridazin-3-yl)-5-pyrazol-1-yl-phenol; 2-{6-[4-(2-hydroxy-ethyl)-piperazin-1-yl]-pyridazin-3-yl}-5-pyrazol-1-yl-phenol; 2-[6-(3,6-diaza-bicyclo[3.2.1]oct-3-yl)-pyridazin-3-yl]-5-pyrazol-1-yl-phenol; 2-[6-(2,7-diaza-spiro[3.5]non-7-yl)-pyridazin-3-yl]-5-pyrazol-1-yl-phenol; 2-[6-(3-hydroxy-methyl-piperazin-1-yl)-pyridazin-3-yl]-5-pyrazol-1-yl-phenol; 2-[6-(1,7-diaza-spiro[4.4]non-7-yl)-pyridazin-3-yl]-5-pyrazol-1-yl-phenol; 2-[6-(4-amino-4-methyl-piperidin-1-yl)-pyridazin-3-yl]-5-pyrazol-1-yl-phenol; 2-[6-(3-dimethyl-amino-piperidin-1-yl)-pyridazin-3-yl]-5-pyrazol-1-yl-phenol; 2-[6-(1,2,2,6,6-pentamethyl-piperidin-4-ylamino)-pyridazin-3-yl]-5-pyrazol-1-yl-phenol; 2-[6-(3,3-dimethyl-piperazin-1-yl)-pyridazin-3-yl]-5-pyrazol-1-yl-phenol; 2-(6-(7-(2-hydroxyethyl)-2,7-diazaspiro[4.4]-nonan-2-yl)pyridazin-3-yl)-5-(1H-pyrazol-1-yl)phenol; 2-(6-((3aR,6aS)-hexahydropyrrolo[3,4-c]pyrrol-2(1H)-yl)pyridazin-3-yl)-5-(1H-pyrazol-1-yl)phenol; 3-(6-(piperazin-1-yl)pyridazin-3-yl)naphthalene-2,7-diol; 5-pyrazol-1-yl-2-[6-(1,2,3,6-tetrahydro-pyridin-4-yl)-pyridazin-3-yl]-phenol; 2-(6-piperidin-4-yl-pyridazin-3-yl)-5-pyrazol-1-yl-phenol; 3-(6-(1,2,3,6-tetra-hydropyridin-4-yl)pyridazin-3-yl)naphthalen-2-ol; 3-(6-(1,2,3,6-tetrahydropyridin-4-yl)pyridazin-3-yl)naphthalene-2,7-diol; 3-(6-(2,2,6,6-tetramethyl-1,2,3,6-tetrahydropyridin-4-yl)pyridazin-3-yl)naphthalene-2,7-diol; 3-(6-(1-methyl-1,2,3,6-tetrahydropyridin-4-yl)pyridazin-3-yl)naphthalene-2,7-diol; 3-(6-(piperidin-4-yl)pyridazin-3-yl)naphthalene-2,7-diol; 3-(6-((2,2,6,6-tetramethylpiperidin-4-yl)oxy)pyridazin-3-yl)naphthalene-2,7-diol; 3-(6-(methyl(2,2,6,6-tetramethylpiperidin-4-yl)amino)pyridazin-3-yl)naphthalene-2,7-diol; 3-(6-((2,2,6,6-tetramethylpiperidin-4-yl)amino)pyridazin-3-yl)naphthalene-2,7-diol; [3-(7-hydroxy-6-{6-[methyl-(2,2,6,6-tetramethyl-piperidin-4-yl)-amino]-pyridazin-3-yl}-naphthalen-2-yloxy)-propyl]-carbamic acid tert-butyl ester; 7-(3-amino-propoxy)-3-{6-[methyl-(2,2,6,6-tetramethyl-piperidin-4-yl)-amino]-pyridazin-3-yl}-naphthalen-2-ol; N-[3-(7-hydroxy-6-{6 [methyl-(2,2,6,6-tetramethyl-piperidin-4-yl)-amino]-pyridazin-3-yl}-naphthalen-2-yloxy)-propyl]-acetamide; 7-(3-hydroxypropoxy)-3-(6-(methyl(2,2,6,6-tetramethylpiperidin-4-yl)amino)pyridazin-3-yl)naphthalen-2-ol; 7-(3-methoxypropoxy)-3-(6-(methyl(2,2,6,6-tetramethylpiperidin-4-yl)amino)pyridazin-3-yl)naphthalen-2-ol; 7-(2-morphobnoethoxy)-3-(6-((2,2,6,6-tetramethylpiperidin-4-yl)oxy)pyridazin-3-yl)naphthalen-2-ol; 3-(6-(piperidin-4-ylmethyl)pyridazin-3-yl)naphthalen-2-ol; 5-(1H-pyrazol-1-yl)-2-(6-((2,2,6,6-tetramethylpiperidin-4-yl)methyl)pyridazin-3-yl)phenol; 3-methoxy-2-(6-(methyl(2,2,6-trimethylpiperidin-4-yl)amino)pyridazin-3-yl)-5-(5-methyloxazol-2-yl)phenol; 2-(6-((6S)-6-((S)-1-hydroxyethyl)-2,2-dimethylpiperidin-4-yloxy)pyridazin-3-yl)-5-(1H-pyrazol-1-yl)phenol; 7-hydroxy-6-(6-(methyl(2,2,6,6-tetramethylpiperidin-4-yl)amino)pyridazin-3-yl)-2-naphthonitrile; 3-(6-(methyl(2,2,6,6-tetramethylpiperidin-4-yl)amino)pyridazin-3-yl)-7-(piperidin-1-ylmethyl)naphthalen-2-ol; 3-(6-(methyl(2,2,6,6-tetramethylpiperidin-4-yl)amino)pyridazin-3-yl)-7-(pyrrobdin-1-ylmethyl)naphthalen-2-ol; 1-bromo-6-(6-(methyl(2,2,6,6-tetramethylpiperidin-4-yl)amino)pyridazin-3-yl)naphthalene-2,7-diol; 1-chloro-6-(6-(methyl(2,2,6,6-tetramethylpiperidin-4-yl)amino)pyridazin-3-yl)naphthalene-2,7-diol; 7-methoxy-3-(6-(methyl(2,2,6,6-tetramethylpiperidin-4-yl)amino)pyridazin-3-yl)naphthalen-2-ol; 7-methoxy-3-(6-(methyl(1,2,2,6,6-pentamethylpiperidin-4-yl)amino)pyridazin-3-yl)naphthalen-2-ol; 7-(3,6-dihydro-2H-pyran-4-yl)-3-(6-(methyl(2,2,6,6-tetramethylpiperidin-4-yl)amino)pyridazin-3-yl)naphthalen-2-ol; 3-(6-(methyl(2,2,6,6-tetramethylpiperidin-4-yl)amino)pyridazin-3-yl)-7-(tetrahydro-2H-pyran-4-yl)naphthalen-2-ol; 7-(difluoromethyl)-3-(6-(methyl(2,2,6,6-tetramethylpiperidin-4-yl)amino)pyridazin-3-yl)naphthalen-2-ol; 7-((4-hydroxy-2-methylbutan-2-yl)oxy)-3-(6-(methyl(2,2,6,6-tetramethylpiperidin-4-yl)amino)pyridazin-3-yl)naphthalen-2-ol; 7-(3-hydroxy-3-methylbutoxy)-3-(6-(methyl(2,2,6,6-tetramethylpiperidin-4-yl)amino)pyridazin-3-yl)naphthalen-2-ol; 2-(6-(methyl(2,2,6,6-tetramethylpiperidin-4-yl)amino)pyridazin-3-yl)-5-(1H-pyrazol-4-yl)benzene-1,3-diol; 3-methoxy-2-(6-(methyl(2,2,6,6-tetramethylpiperidin-4-yl)amino)pyridazin-3-yl)-5-(1H-pyrazol-4-yl)phenol; 5-(1H-pyrazol-4-yl)-2-(6-((2,2,6,6-tetramethylpiperidin-4-yl)amino)pyridazin-3-yl)-3-(trifluorc>methoxy)phenol; 2-(6-(methyl(2,2,6,6-tetramethylpiperidin-4-yl)amino)pyridazin-3-yl)-5-(1-methyl-1H-pyrazol-4-yl)-3-(trifluoromethoxy)phenol; 2-(6-(methyl(2,2,6,6-tetramethylpiperidin-4-yl)amino)pyridazin-3-yl)-5-(1H-pyrazol-4-yl)-3-(trifluoromethoxy)phenol; 4-(3-hydroxy-4-(6-(methyl(2,2,6,6-tetramethylpiperidin-4-yl)amino)pyridazin-3-yl)-5-(trifluoromethoxy)phenyl)-1-methylpyridin-2(1H)-one; 3-methoxy-2-(6-(methyl(2,2,6,6-tetramethylpiperidin-4-yl)amino)pyridazin-3-yl)-5-(1-methyl-1H-pyrazol-4-yl)phenol; 3-methoxy-2-(6-(methyl(2,2,6,6-tetramethylpiperidin-4-yl)amino)pyridazin-3-yl)-5-(5,6,7,8-tetrahydroimidazo[1,2-a]pyridin-3-yl)phenol; 3-methoxy-2-(6-(methyl(2,2,6,6-tetramethylpiperidin-4-yl)amino)pyridazin-3-yl)-5-(pyridin-3-yl)phenol; 5-(1-cyclopentyl-1H-pyrazol-4-yl)-3-methoxy-2-(6-(methyl(2,2,6,6-tetramethylpiperidin-4-yl)amino)pyridazin-3-yl)phenol; 3′,5-dimethoxy-4-(6-(methyl(2,2,6,6-tetramethylpiperidin-4-yl)amino)pyridazin-3-yl)-[l,r-biphenyl]-3-ol; 3-(benzyloxy)-2-(6-(methyl(2,2,6,6-tetramethylpiperidin-4-yl)amino)pyridazin-3-yl)-5-(5-methyloxazol-2-yl)phenol; 3-ethoxy-2-(6-(methyl(2,2,6,6-tebamethylpiperidin-4-yl)amino)pyridazin-3-yl)-5-(5-methyloxazol-2-yl)phenol; 3-(cyclopropylmethoxy)-2-(6-(methyl(2,2,6,6-tetramethylpiperidin-4-yl)amino)-pyridazin-3-yl)-5-(5-methyloxazol-2-yl)phenol; 2-methyl-5-(6-(methyl(2,2,6,6-tetramethylpiperidin-4-yl)amino)pyridazin-3-yl)-1H-benzo[d]imidazol-6-ol; 5-chloro-2-(6-(methyl(2,2,6,6-tetramethylpiperidin-4-yl)amino)pyridazin-3-yl)phenol; 5-(1H-pyrazol-1-yl)-2-(6-((2,2,6,6-tetramethylpiperidin-4-yl)amino)pyridazin-3-yl)phenol; 3-hydroxy-4-(6-((2,2,6,6-tetramethylpiperidin-4-yl)amino)pyridazin-3-yl)benzonitrile; 2-(6-((2,2-dimethylpiperidin-4-yl)oxy)pyridazin-3-yl)-5-(1H-pyrazol-1-yl)phenol; 2-(6-(methyl(2,2,6,6-tetramethylpiperidin-4-yl)amino)pyridazin-3-yl)-4-(1H-pyrazol-4-yl)phenol; 2-(6-(methyl(2,2,6,6-tetramethylpiperidin-4-yl)amino)pyridazin-3-yl)-4-(4,5,6,7-tetrahydropyrazolo[1,5-a]pyridin-3-yl)phenol; 2-(6-(methyl(2,2,6,6-tetramethylpiperidin-4-yl)amino)pyridazin-3-yl)-4-(4,5,6,7-tetrahydropyrazolo[1,5-a]pyrazin-3-yl)phenol; 4-(1H-indol-2-yl)-2-(6-(methyl(2,2,6,6-tetramethylpiperidin-4-yl)amino)pyridazin-3-yl)phenol; 4-(cyclopent-1-en-1-yl)-2-(6-(methyl(2,2,6,6-tetramethylpiperidin-4-yl)amino)pyridazin-3-yl)phenol; 2-(6-(methyl(2,2,6,6-tetramethylpiperidin-4-yl)amino)pyridazin-3-yl)-4-(1H-pyrazol-3-yl)phenol; 4-(4-hydroxy-3-(6-(methyl(2,2,6,6-tetramethylpiperidin-4-yl)amino)pyridazin-3-yl)phenyl)pyridin-2-ol; 4-(4-hydroxy-3-(6-((2,2,6,6-tetramethylpiperidin-4-yl)oxy)pyridazin-3-yl)phenyl)-1-methylpyridin-2(1H)-one; 4-(4-hydroxy-3-(6-((2,2,6,6-tetramethylpiperidin-4-yl)oxy)pyridazin-3-yl)phenyl)pyridin-2-ol; 5-(1H-indazol-7-yl)-2-(6-(methyl(2,2,6,6-tetramethylpiperidin-4-yl)amino)pyridazin-3-yl)phenol; 4-chloro-2-(6-(methyl(2,2,6,6-tetramethylpiperidin-4-yl)amino)pyridazin-3-yl)-5-(1H-pyrazol-4-yl)phenol; 4-fluoro-2-(6-(methyl(2,2,6,6-tetramethylpiperidin-4-yl)amino)pyridazin-3-yl)-5-(1H-pyrazol-4-yl)phenol; 5-fluoro-4-(1H-imidazol-4-yl)-2-(6-(methyl(2,2,6,6-tetramethylpiperidin-4-yl)amino)pyridazin-3-yl)phenol; 5-fluoro-2-(6-(methyl(2,2,6,6-tetramethylpiperidin-4-yl)amino)pyridazin-3-yl)-4-(1H-pyrazol-4-yl)phenol; 5-fluoro-2-(6-(methyl(2,2,6,6-tetramethylpiperidin-4-yl)amino)pyridazin-3-yl)-4-(1H-pyrazol-5-yl)phenol; 6-hydroxy-5-(6-(methyl(2,2,6,6-tetramethylpiperidin-4-yl)amino)pyridazin-3-yl)-2,3-dihydro-1H-inden-1-one; 6-(6-(methyl(2,2,6,6-tetramethylpiperidin-4-yl)amino)pyridazin-3-yl)-1,4-dihydroindeno[1,2-c]pyrazol-7-ol; 6-hydroxy-5-(6-(methyl(2,2,6,6-tetramethylpiperidin-4-yl)amino)pyridazin-3-yl)-2,3-dihydro-1H-inden-1-one oxime hydrochloride salt; 5-(6-(methyl(2,2,6,6-tetramethylpiperidin-4-yl)amino)pyridazin-3-yl)-2,3-dihydro-1H-indene-1,6-diol; 2-amino-6-(6-(methyl(2,2,6,6-tetramethylpiperidin-4-yl)amino)pyridazin-3-yl)-8H-indeno[1,2-d]thiazol-5-ol hydrochloride salt; 9-(6-(methyl(2,2,6,6-tetramethylpiperidin-4-yl)amino)pyridazin-3-yl)-5,6-dihydroimidazo[5,1-a]isoquinolin-8-ol hydrochloride salt; 4-hydroxy-3-(6-(methyl(2,2,6,6-tetramethylpiperidin-4-yl)amino)pyridazin-3-yl)-N-((1-methyl-1H-pyrazol-4-yl)methyl)benzamide; 4-(4-(hydroxymethyl)-1H-pyrazol-1-yl)-2-(6-(methyl(2,2,6,6-tetramethylpiperidin-4-yl)amino)pyridazin-3-yl)phenol; 5-(1H-pyrazol-4-yl)-2-(6-((2,2,6,6-tetramethylpiperidin-4-yl)methyl)pyridazin-3-yl)phenol; 6-(3-(benzyloxy) isoquinolin-6-yl)-N-methyl-N-(2,2,6,6-tetramethylpiperidin-4-yl)pyridazin-3-amine; 6-(1-(benzyloxy) isoquinolin-7-yl)-N-methyl-N-(2,2,6,6-tetramethylpiperidin-4-yl)pyridazin-3-amine; 3-fluoro-5-(2-methoxypyridin-4-yl)-2-(6-(methyl(2,2,6,6-tetramethylpiperidin-4-yl)amino)pyridazin-3-yl)phenol hydrochloride salt; 4-(3-fluoro-5-hydroxy-4-(6-(methyl(2,2,6,6-tetramethylpiperidin-4-yl)amino)pyridazin-3-yl)phenyl)pyridin-2(1H)-one hydrochloride salt; 4-(3-fluoro-5-hydroxy-4-(6-(methyl(2,2,6,6-tetramethylpiperidin-4-yl)amino)pyridazin-3-yl)phenyl)-1-methylpyridin-2(1H)-one hydrochloride salt; 5-(3-fluoro-5-hydroxy-4-(6-(methyl(2,2,6,6-tetramethylpiperidin-4-yl)amino)pyridazin-3-yl)phenyl)-1-methylpyridin-2(1H)-one hydrochloride salt; 3-fluoro-5-(1H-pyrazol-4-yl)-2-(6-((2,2,6,6-tetramethylpiperidin-4-yl)oxy)pyridazin-3-yl)phenol hydrochloride salt; 5-chloro-3-fluoro-2-(6-(methyl(2,2,6,6-tetramethylpiperidin-4-yl)amino)pyridazin-3-yl)phenol hydrochloride salt; 3-fluoro-2-(6-(methyl(2,2,6,6-tetramethylpiperidin-4-yl)amino)pyridazin-3-yl)-5-(1H-pyrazol-4-yl)phenol hydrochloride salt; 3-fluoro-2-(6-(methyl(2,2,6,6-tetramethylpiperidin-4-yl)amino)pyridazin-3-yl)-5-(1-methyl-1H-pyrazol-4-yl)phenol hydrochloride salt; 5-(5-methoxypyridin-3-yl)-2-(6-(methyl(2,2,6,6-tetramethylpiperidin-4-yl)amino)pyridazin-3-yl)phenol; 5-(3-hydroxy-4-(6-methyl(2,2,6,6-tetramethylpiperidin-4-yl)amino)pyridazin-3-yl)phenyl)pyridin-2-ol; 4-(3-hydroxy-4-(6-methyl(2,2,6,6-tetramethylpiperidin-4-yl)amino)pyridazin-3-yl)phenyl)pyridin-2-ol; 5-(6-methoxypyridin-3-yl)-2-(6-(methyl(2,2,6,6-tetramethylpiperidin-4-yl)amino)pyridazin-3-yl)phenol; 5-(3-hydroxy-4-(6-(methyl(2,2,6,6-tetramethylpiperidin-4-yl)amino)pyridazin-3-yl)phenyl)-3-(trifluoromethyl)pyridin-2-ol; 5-(3-hydroxy-4-(6-(methyl(2,2,6,6-tetramethylpiperidin-4-yl)amino)pyridazin-3-yl)phenyl)-1-methylpyridin-2 (1H)-one; 4-(3-hydroxy-4-(6-(methyl(2,2,6,6-tetramethylpiperidin-4-yl)amino)pyridazin-3-yl)phenyl)-1-methylpyridin-2(1H)-one; 5-(2-methoxypyridin-4-yl)-2-(6-(methyl(2,2,6,6-tetramethylpiperidin-4-yl)amino)pyridazin-3-yl)phenol; 4-(3-hydroxy-4-(6-((2,2,6,6-tetramethylpiperidin-4-yl)oxy)pyridazin-3-yl)phenyl)pyridin-2-ol; 5-(6-(dimethylamino)pyridin-3-yl)-2-(6-(methyl(2,2,6,6-tetramethylpiperidin-4-yl)amino)pyridazin-3-yl)phenol; 4-(3-hydroxy-4-(6-((2,2,6,6-tetramethylpiperidin-4-yl)oxy)pyridazin-3-yl)phenyl)-1-methylpyridin-2(1H)-one; 2-(6-(methyl(2,2,6,6-tetramethylpiperidin-4-yl)amino)pyridazin-3-yl)-5-(pyrimidin-5-yl)phenol; 5-(3-hydroxy-4-(6-(methyl(2,2,6,6-tetramethylpiperidin-4-yl)amino)pyridazin-3-yl)phenyl)pyridin-3-ol; 1-cy clopropyl-4-(3-hydroxy-4-(6-(methyl(2,2,6,6-tetramethylpiperidin-4-yl)amino)pyridazin-3-yl)phenyl)pyridin-2(1H)-one; 2-(6-(methyl(2,2,6,6-tetramethylpiperidin-4-yl)amino)pyridazin-3-yl)-5-(1,2,3,6-tetrahydropyridin-4-yl)phenol; 5-(cyclopent-1-en-1-yl)-2-(6-(methyl(2,2,6,6-tetramethylpiperidin-4-yl)amino)pyridazin-3-yl)phenol; 5-(3,6-dihydro-2H-pyran-4-yl)-2-(6-(methyl(2,2,6,6-tetramethylpiperidin-4-yl)amino)pyridazin-3-yl)phenol; 5-(imidazo[1,5-a]pyridin-7-yl)-2-(6-(methyl(2,2,6,6-tetramethylpiperidin-4-yl)amino)pyridazin-3-yl)phenol; 5-(imidazo[1,2-a]pyridin-7-yl)-2-(6-(methyl(2,2,6,6-tetramethylpiperidin-4-yl)amino)pyridazin-3-yl)phenol; 2-(6-(methyl(2,2,6,6-tetramethylpiperidin-4-yl)amino)pyridazin-3-yl)-5-(2-methylpyridin-4-yl)phenol; 5-(1H-imidazol-2-yl)-2-(6-(methyl(2,2,6,6-tetramethylpiperidin-4-yl)amino)pyridazin-3-yl)phenol; 5-(1H-imidazol-4-yl)-2-(6-(methyl(2,2,6,6-tetramethylpiperidin-4-yl)amino)pyridazin-3-yl)phenol; 5-(imidazo[1,2-a]pyrazin-3-yl)-2-(6-(methyl(2,2,6,6-tetramethylpiperidin-4-yl)amino)pyridazin-3-yl)phenol; 2-(6-(methyl(2,2,6,6-tetramethylpiperidin-4-yl)amino)pyridazin-3-yl)-5-(5,6,7,8-tetrahydroimidazo[1,2-a]pyrazin-3-yl)phenol; 2-(6-(methyl(2,2,6,6-tetramethylpiperidin-4-yl)amino)pyridazin-3-yl)-5-(4-methyl-1H-imidazol-2-yl)phenol; 2-(6-(methyl(2,2,6,6-tetramethylpiperidin-4-yl)amino)pyridazin-3-yl)-5-(1-methyl-1H-imidazol-4-yl)phenol; 2-(6-(methyl(2,2,6,6-tetramethylpiperidin-4-yl)amino)pyridazin-3-yl)-5-(1-methyl-1H-imidazol-5-yl)phenol; 2-(6-(methyl(2,2,6,6-tetramethylpiperidin-4-yl)amino)pyridazin-3-yl)-5-(4-nitro-1H-imidazol-2-yl)phenol; 2-(6-(methyl(2,2,6,6-tetramethylpiperidin-4-yl)amino)pyridazin-3-yl)-5-(2-methyl-1H-imidazol-4-yl)phenol; 5-(1,2-dimethyl-1H-imidazol-4-yl)-2-(6-(methyl(2,2,6,6-tetramethylpiperidin-4-yl)amino)pyridazin-3-yl)phenol; 1-(3-hydroxy-4-(6-(methyl(2,2,6,6-tetramethylpiperidin-4-yl)amino)pyridazin-3-yl)phenyl)-1H-pyrazole-4-carboxamide; 2-(6-((3aR,6aS)-5-(2-hydroxyethyl) hexahydropyrrolo[3,4-c]pyrrol-2(1H)-yl)pyridazin-3-yl)-5-(1H-pyrazol-4-yl)phenol; 2-(6-((3aR,6aS)-hexahydropyrrolo[3,4-c]pyrrol-2(1H)-yl)pyridazin-3-yl)-5-(1H-pyrazol-4-yl)phenol; 2-(6-((3aR,6aS)-5-methylhexahydropyrrolo[3,4-c]pyrrol-2(1H)-yl)pyridazin-3-yl)-5-(1H-pyrazol-4-yl)phenol; 4-(3-hydroxy-4-(6-(5-methylhexahydropyrrolo[3,4-c]pynOl-2(1H)-yl)pyridazin-3-yl)phenyl)-1-methylpyridin-2(1H)-one; 4-(3-hydroxy-4-(6-((3aR,6aR)-1-methylhexahydropyrrolo[3,4-b]pyrrol-5(1H)-yl)pyridazin-3-yl)phenyl)-1-methylpyridin-2(1H)-one; 2-(6-(2,7-diazaspiro[4.5]decan-2-yl)pyridazin-3-yl)-5-(1H-pyrazol-4-yl)phenol; and 4-(4-(6-(2,7-diazaspiro[4.5]decan-2-yl)pyridazin-3-yl)-3-hydroxyphenyl)-1-methylpyridin-2(1H)-one.

In some embodiments, the small molecule splice modulator is RG7916 (Roche/PTC/SMAF, 7-(4,7-diazaspiro[2.5]octan-7-yl)-2-(2,8-dimethylimidazo[1,2-b]pyridazin-6-yl)-4H-pyrido[1,2-a]pyrimidin-4-one) (CAS No.: 1825352-65-5) having the following structure:

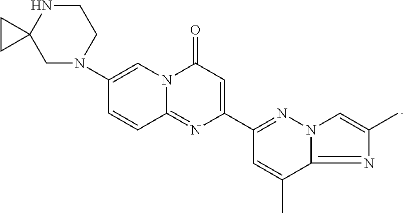 [SMILES: Cc1cn2nc(-c3cc(=O)n4cc(N5CCNC6(CC6)C5)ccc4n3)cc(C)c2n1]

In some embodiments, the small molecule splice modulator is RG7800 (Roche) (CAS No.: 1449598-06-4) having the following structure:

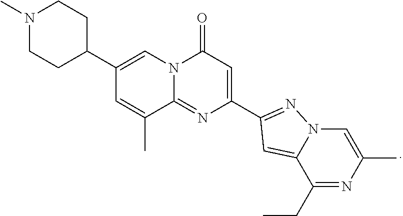

In some embodiments, the small molecule splice modulator is an analogue of RG7916 or RG7800. Exemplary analogues of RG7916 and RG7800 include, but are not limited to, 2-(2-methylimidazo[1,2-b]pyridazin-6-yl)-7-(4-methylpiperazin-1-yl)pyrido[1,2-a]pyrimidin-4-one; 7-[(8aR)-3,4,6,7,8,8a-hexahydro-1H-pyrrolo[1,2-a]pyrazin-2-y 1]-2-(2-methylimidazo[1,2-b]pyridazin-6-yl)pyrido[1,2-a]pyrimidin-4-one; 7-[(8aS)-3,4,6,7,8,8a-hexahydro-1H-pyrrolo[1,2-a]pyrazin-2-yl]-2-(2,8-dimethylimidazo[1,2-b]pyridazin-6-yl)pyrido[1,2-a]pyrimidin-4-one; 7-[(8aR)-3,4,6,7,8,8a-hexahydro-1H-pyrrolo[1,2-a]pyrazin-2-yl]-2-(2,8-dimethylimidazo[1,2-b]pyridazin-6-yl)pyrido[1,2-a]pyrimidin-4-one; 7-[(8aS)-8a-methyl-1,3,4,6,7,8-hexahydropyrrolo[1,2-a]pyrazin-2-yl]-2-(2,8-dimethylimidazo[1,2-b]pyridazin-6-yl)pyrido[1,2-a]pyrimidin-4-one; 7-[(8aR)-8a-methyl-1,3,4,6,7,8-hexahydropyrrolo[1,2-a]pyrazin-2-yl]-2-(2,8-dimethylimidazo[1,2-b]pyridazin-6-yl)pyrido[1,2-a]pyrimidin-4-one; 2-(2,8-dimethylimidazo[1,2-b]pyridazin-6-yl)-7-[(3S,5R)-3,5-dimethylpiperazin-1-yl]pyrido[1,2-a]pyrimidin-4-one; 2-(2,8-dimethylimidazo[1,2-b]pyridazin-6-yl)-7-[(3S)-3-methylpiperazin-1-yl]pyrido[1,2-a]pyrimidin-4-one; 2-(2,8-dimethylimidazo[1,2-b]pyridazin-6-yl)-7-[(3R)-3-methylpiperazin-1-yl]pyrido[1,2-a]pyrimidin-4-one; 7-(1,4-diazepan-1-yl)-2-(2,8-dimethylimidazo[1,2-b]pyridazin-6-yl)pyrido[1,2-a]pyrimidin-4-one; 2-(2-methyhmidazo[1,2-b]pyridazin-6-yl)-7-[(3S)-3-methylpiperazin-1-yl]pyrido[1,2-a]pyrimidin-4-one; 2-(2-methylimidazo[1,2-b]pyridazin-6-yl)-7-[(3R)-3-methylpiperazin-1-yl]pyrido[1,2-a]pyrimidin-4-one; 7-(1,4-diazepan-1-yl)-2-(2-methylimidazo[1,2-b]pyridazin-6-yl)pyrido[1,2-a]pyrimidin-4-one; 7-[(3R,5S)-3,5-dimethylpiperazin-1-yl]-2-(2-methylimidazo[1,2-b]pyridazin-6-yl)pyrido[1,2-a]pyrimidin-4-one; 7-[(8aS)-3,4,6,7,8,8a-hexahydro-1H-pyrrolo[1,2-a]pyrazin-2-y 1]-2-(2-methylimidazo[1,2-b]pyridazin-6-yl)pyrido[1,2-a]pyrimidin-4-one; 7-[(8aS)-8a-methyl-1,3,4,6,7,8-hexahydropyrrolo[1,2-a]pyrazin-2-yl]-2-(2-methylimidazo[1,2-b]pyridazin-6-yl)pyrido[1,2-a]pyrimidin-4-one; 7-[(8aR)-8a-methyl-1,3,4,6,7,8-hexahydropyrrolo[1,2-a]pyrazin-2-yl]-2-(2-methylimidazo[1,2-b]pyridazin-6-yl)pyrido[1,2-a]pyrimidin-4-one; 2-(2,8-dimethylimidazo[1,2-b]pyridazin-6-yl)-7-[(3R)-3-pyrrobdin-1-ylpynOlidin-1-yl]pyrido[1,2-a]pyrimidin-4-one; 7-(4,7-diazaspiro[2.5]octan-7-yl)-2-(2-methybmidazo[1,2-b]pyridazin-6-yl)pyrido[1,2-a]pyrimidin-4-one; 7-(4,7-diazaspiro[2.5]octan-7-yl)-2-(2,8-dimethybmidazo[1,2-b]pyridazin-6-yl)pyrido[1,2-a]pyrimidin-4-one; 2-(2-methylimidazo[1,2-b]pyridazin-6-yl)-7-[(3R)-3-pyrrobdin-1-ylpynObdin-1-yl]pyrido[1,2-a]pyrimidin-4-one; 2-(2,8-dimethylimidazo[1,2-b]pyridazin-6-yl)-7-(3,3-dimethylpiperazin-1-yl)pyrido[1,2-a]pyrimidin-4-one; 7-(3,3-dimethylpiperazin-1-yl)-2-(2-methybmidazo[1,2-b]pyridazin-6-yl)pyrido[1,2-a]pyrimidin-d one; 2-(2,8-dimethylimidazo[1,2-b]pyridazin-6-yl)-9-methyl-7-[(3S)-3-methylpiperazin-1-yl]pyrido[1,2-a]pyrimidin-4-one; 2-(2,8-dimethylimidazo[1,2-b]pyridazin-6-yl)-9-methyl-7-[(3R)-3-methylpiperazin-1-yl]pyrido[1,2-a]pyrimidin-4-one; 2-(2,8-dimethylimidazo[1,2-b]pyridazin-6-yl)-7-[(3R,5S)-3,5-dimethylpiperazin-1-yl]-9-methyl-pyrido[1,2-a]pyrimidin-4-one; 2-(2,8-dimethylimidazo[1,2-b]pyridazin-6-yl)-7-(3,3-dimethylpiperazin-1-yl)-9-methyl-pyrido[1,2-a]pyrimidin-4-one; 7-(4,7-diazaspiro[2.5]octan-7-yl)-2-(2,8-dimethybmidazo[1,2-b]pyridazin-6-yl)-9-methyl-pyrido[1,2-a]pyrimidin-4-one; 2-(2,8-dimethybmidazo[1,2-b]pyridazin-6-yl)-7-[(3S,5S)-3,5-dimethylpiperazin-1-yl]pyrido[1,2-a]pyrimidin-4-one; 2-(2,8-dimethylimidazo[1,2-b]pyridazin-6-yl)-7-[(3S)-3-pynObdin-1-ylpyrrolidin-1-yl]pyrido[1,2-a]pyrimidin-4-one; 2-(2-methylimidazo[1,2-b]pyridazin-6-yl)-7-[(3S)-3-pyrrobdin-1-ylpynObdin-1-yl]pyrido[1,2-a]pyrimidin-4-one; 7-[(3S,5S)-3,5-dimethylpiperazin-1-yl]-2-(2-methybmidazo[1,2-b]pyridazin-6-yl)pyrido[1,2-a]pyrimidin-4-one; 9-methyl-2-(2-methybmidazo[1,2-b]pyridazin-6-yl)-7-[(3S)-3-methylpiperazin-1-yl]pyrido[1,2-a]pyrimidin-4-one; 9-methyl-2-(2-methybmidazo[1,2-b]pyridazin-6-yl)-7-[(3R)-3-methylpiperazin-1-yl]pyrido[1,2-a]pyrimidin-4-one; 7-[(3R,5S)-3,5-dimethylpiperazin-1-yl]-9-methyl-2-(2-methybmidazo[1,2-b]pyridazin-6-yl)pyrido[1,2-a]pyrimidin-4-one; 7-(3,3-dimethylpiperazin-1-yl)-9-methyl-2-(2-methybmidazo[1,2-b]pyridazin-6-yl)pyrido[1,2-a]pyrimidin-4-one; 7-(4,7-diazaspiro[2.5]octan-7-yl)-9-methyl-2-(2-methybmidazo[1,2-b]pyridazin-6-yl)pyrido[1,2-a]pyrimidin-4-one; 7-[(3S,5S)-3,5-dimethylpiperazin-1-yl]-9-methyl-2-(2-methybmidazo[1,2-b]pyridazin-6-yl)pyrido[1,2-a]pyrimidin-4-one; and 7-[(3R)-3-ethylpiperazin-1-y1]-2-(2-methybmidazo[1,2-b]pyridazin-6-yl)pyrido[1,2-a]pyrimidin-4-one; 7-[(8aS)-3,4,6,7,8,8a-hexahydro-1H-pyrrolo[1,2-a]pyrazin-2-yl]-2-(2,8-dimethybmidazo[1,2-b]pyridazin-6-yl)pyrido[1,2-a]pyrimidin-4-one; 7-[(8aR)-3,4,6,7,8,8a-hexahydro-1H-pyrrolo[1,2-a]pyrazin-2-yl]-2-(2,8-dimethybmidazo[1,2-b]pyridazin-6-yl)pyrido[1,2-a]pyrimidin-4-one; 2-(2,8-dimethylimidazo[1,2-b]pyridazin-6-yl)-7-[(3S,5R)-3,5-dimethylpiperazin-1-yl]pyrido[1,2-a]pyrimidin-4-one; 7-[(3R,5S)-3,5-dimethylpiperazin-1-yl]-2-(2-methylimidazo[1,2-b]pyridazin-6-yl)pyrido[1,2-a]pyrimidin-4-one; 7-[(8aS)-3,4,6,7,8,8a-hexahydro-1H-pyrrolo[1,2-a]pyrazin-2-yl]-2-(2-methylimidazo[1,2-b]pyridazin-6-yl)pyrido[1,2-a]pyrimidin-4-one; 2-(2,8-dimethylimidazo[1,2-b]pyridazin-6-yl)-9-methy 1-7-[(3S)-3-methylpiperazin-1-yl]pyrido[1,2-a]pyrimidin-4-one; 7-fluoro-2-(2-methylimidazo[1,2-b]pyridazin-6-yl)pyrido[1,2-a]pyrimidin-4-one; 2-(2,8-dimethylimidazo[1,2-b]pyridazin-6-yl)-7-fluoro-pyrido[1,2-a]pyrimidin-4-one; 7-fluoro-9-methyl-2-(2-methylimidazo[1,2-b]pyridazin-6-yl)pyrido[1,2-a]pyrimidin-4-one; 2-(2,8-dimethylimidazo[1,2-b]pyridazin-6-yl)-7-fluoro-9-methyl-pyrido[1,2-a]pyrimidin-4-one; or a pharmaceutically acceptable salt thereof.

In some embodiments, the small molecule splice modulator is a sudemycin selected from the group consisting of: (5′,Z)-5-(((1R,4R)-4-((2JE′,4JE)-5-((3R,55′)-7,7-dimethyl-1,6-dioxaspiro[2.5]octan-5-yl)-3-methylpenta-2,4-dien-1-yl)cyclohexyl)amino)-5-oxopent-3-en-2-yl methylcarbamate and (5′,Z)-5-(((1R,4R)-4-((2JE′,4JE)-5-((3R,55′)-7,7-dimethyl-1,6-dioxaspiro[2.5]octan-5-yl)-3-methylpenta-2,4-dien-1-yl)cyclohexyl)amino)-5-oxopent-3-en-2-yl dimethylcarbamate.

In some embodiments, the small molecule splice modulator is a pladienolide compound, including those disclosed in the following patent applications: WO 2002/060890; WO 2004/011459; WO 2004/011661; WO 2004/050890; WO 2005/052152; WO 2006/009276; WO 2008/126918; and WO 2015/175594, each of which are incorporated herein by reference. One example, of a pladienolide compound is (8E,12E,14E)-7-((4-Cycloheptylpiperazin-1-yl) carbonyl)oxy-3,6,16,21-tetrahydroxy-6,10,12,16,20-pentamethyl-18,19-epoxytricosa-8,12,14-trien-11-olide, also known as E7107, which is a semisynthetic derivative of the natural product pladienolide D.

In some embodiments, the minigene is regulated by a disease state in a cell. In some embodiments, the disease state is cancer. In some embodiments, the cancer is glioblastoma.

In some embodiments, the minigene is regulated by a cell type or tissue type.

In some embodiments, the nucleic acids described herein comprise one or moreinterferons (i.e., payloads). In some embodiments, the nucleic acids described herein further comprise a third polynucleotide comprising a minigene 5′ of a fourth polynucleotide encoding an interferon. In some embodiments, the nucleic acids described herein further comprise a fifth polynucleotide comprising a minigene 5′ of a sixth polynucleotide encoding an interferon. In some embodiments, the interferons are the same. In some embodiments, the interferons are different.

In some embodiments, payloads (i.e., one or more interferons and/or a report) are modified such that the payload has ATG and/or a potential alternative in-frame start codon removed, while the protein sequence of the payload is unchanged.

In some embodiments, the interferon (IFN) is a Type I IFN, a Type II IFN, or a Type III IFN. In some embodiments, the IFN is IFN alpha (IFNα), IFN beta (IFNβ), IFN gamma (IFNγ), IFN epsilon (IFNε), IFN kappa (IFNκ), IFN omega (IFNω), IFN lambda (IFNλ), or derivatives thereof. In some embodiments, the interferon is IFNα, IFNβ, IFNγ, or combinations thereof. In some embodiments, the nucleic acids described herein comprise IFNα, IFNβ, IFNγ, IFNε, IFNκ, IFNω, IFNλ, or combinations thereof. In some embodiments, the nucleic acids described herein comprise IFNα, IFNβ, IFNγ, IFNα-IFNβ, IFNα-IFNγ, IFNβ-IFNγ, and IFNα-IFNβ-IFNγ. In some embodiments, the interferon is a subtype of IFNα. In some embodiments, the IFNα is IFNα1, IFNα2, IFNα4, IFNα5, IFNα6, IFNα7, IFNα8, IFNα10, IFNα13, IFNα14, IFNα16, IFNα17, IFNα21, or derivatives thereof.

In some embodiments, the IFN is mouse, rat, equine, sheep, cow, primate (e.g., chimpanzee, baboon, gorilla, orangutan, monkey), dog, cat, pig, donkey, rabbit, fish, fly, or human. In some embodiments, the nucleic acids described herein comprise IFNα, IFNβ, IFNγ, IFNα-IFNβ, IFNα-IFNγ, IFNβ-IFNγ, and IFNα-IFNβ-IFNγ and the IFN is mouse. In some embodiments, the nucleic acids described herein comprise IFNα, IFNβ, IFNγ, IFNα-IFNβ, IFNα-IFNγ, IFNβ-IFNγ, and IFNα-IFNβ-IFNγ and the IFN is human. In some embodiments, the nucleic acids described herein comprise a combination of human or mouse IFNα, IFNβ, IFNγ, IFNα-IFNβ, IFNα-IFNγ, IFNβ-IFNγ, and IFNα-IFNβ-IFNγ. In some embodiments, the nucleic acids described herein comprise human IFNα, human IFNβ, human IFNγ, or combinations thereof. In some embodiments, the nucleic acids described herein comprise mouse IFNα, mouse IFNβ, mouse IFNγ, or combinations thereof. In some embodiments, the nucleic acids described herein comprise human IFNα, human IFNβ, human IFNγ, mouse IFNα, mouse IFNβ, mouse IFNγ, or combinations thereof.

In some embodiments, the nucleic acids encoding one or more interferons comprise reduced CpG dinucleotides.

In some embodiments, the CpG dinucleotides are reduced by at least about 5%, 10%, 15%, 20%, 25%, 30%, 35%, 40%, 45%, 50%, 55%, 60%, 65%, 70%, 75%, 80%, 85%, 90%, 95%, or more than 95% as compared to a parental equivalent. In some embodiments, the CpG dinucleotides are reduced in a range of about 5% to about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, the CpG dinucleotides are reduced in a range of about 10% to about 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, the CpG dinucleotides are reduced in a range of about 15% to about 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, the CpG dinucleotides are reduced in a range of about 20% to about 25%, 30%, 35%, 40%, 45%, 50%, 55%, 60%, 65%, 70%, 75%, 80%, 85%, 90%, 95%, 96%, 97%, 98%, 99%, or 100%. In some embodiments, the CpG dinucleotides are reduced in a range of about 25% to about 30%, 35%, 40%, 45%, 50%, 55%, 60%, 65%, 70%, 75%, 80%, 85%, 90%, 95%, 96%, 97%, 98%, 99%, or 100%. In some embodiments, the CpG dinucleotides are reduced in a range of about 30% to about 35%, 40%, 45%, 50%, 55%, 60%, 65%, 70%, 75%, 80%, 85%, 90%, 95%, 96%, 97%, 98%, 99%, or 100%. In some embodiments, the CpG dinucleotides are reduced in a range of about 40% to about 45%, 50%, 55%, 60%, 65%, 70%, 75%, 80%, 85%, 90%, 95%, 96%, 97%, 98%, 99%, or 100%. In some embodiments, the CpG dinucleotides are reduced in a range of about 50% to about 55%, 60%, 65%, 70%, 75%, 80%, 85%, 90%, 95%, 96%, 97%, 98%, 99%, or 100%. In some embodiments, the CpG dinucleotides are reduced in a range of about 60% to about 65%, 70%, 75%, 80%, 85%, 90%, 95%, 96%, 97%, 98%, 99%, or 100%. In some embodiments, the CpG dinucleotides are reduced in a range of about 70% to about 75%, 80%, 85%, 90%, 95%, 96%, 97%, 98%, 99%, or 100%. In some embodiments, the CpG dinucleotides are reduced in a range of about 80% to about 85%, 90%, 95%, 96%, 97%, 98%, 99%, or 100%. In some embodiments, the CpG dinucleotides are reduced in a range of about 90% to about 95%, 96%, 97%, 98%, 99%, or 100%. In some embodiments, the CpG dinucleotides are reduced by at least about 50%. In some embodiments, the CpG dinucleotides are reduced by at least about 75%.

In some embodiments, the nucleic acid sequences are codon optimized for expression of the interferon. Codon optimization can be used to increase the rate of translation or to produce recombinant RNA transcripts having desirable properties, such greater expression efficiency, as compared with transcripts produced using a non-optimized sequence. In particular embodiments, the nucleic acid sequences are codon optimized for expression in human cells. Methods for codon optimization are readily available, for example, optimizer, accessible free of charge at http://genomes.urv.es/OPTIMIZER, and GeneGPS® Expression Optimization Technology from DNA 2.0 (Newark, California). See Raab et al., Syst Synth Biol 4:215 (2010).

In some embodiments, the human interferon (hINF) beta (hIFNβ) has the sequence represented by UniProt/SwissProt Database Entry No. P01574 (SEQ ID NO: 1). In some embodiments, the hIFNβ is encoded by the nucleic acid sequence of any one of SEQ ID NOs: 2-4. In some embodiments, the hIFNβ is encoded by a nucleic acid sequence having at least 70% (e.g., 75%, 80%, 90%, 95%, 96%, 97%, 98%, or 99%) sequence identity to the nucleic acid sequence of any one of SEQ ID NOs: 2-4. In some embodiments, the hIFNβ is encoded by a nucleic acid sequence having at least 75% sequence identity to the nucleic acid sequence of any one of SEQ ID NOs: 2-4. In some embodiments, the hIFNβ is encoded by a nucleic acid sequence having at least 80% sequence identity to the nucleic acid sequence of any one of SEQ ID NOs: 2-4. In some embodiments, the hIFNβ is encoded by a nucleic acid sequence having at least 90% sequence identity to the nucleic acid sequence of any one of SEQ ID NOs: 2-4. In some embodiments, the hIFNβ is encoded by a nucleic acid sequence having at least 95% sequence identity to the nucleic acid sequence of any one of SEQ ID NOs: 2-4. In some embodiments, the hIFNβ is encoded by a nucleic acid sequence having at least 96% sequence identity to the nucleic acid sequence of any one of SEQ ID NOs: 2-4. In some embodiments, the hIFNβ is encoded by a nucleic acid sequence having at least 97% sequence identity to the nucleic acid sequence of any one of SEQ ID NOs: 2-4. In some embodiments, the hIFNβ is encoded by a nucleic acid sequence having at least 98% sequence identity to the nucleic acid sequence of any one of SEQ ID NOs: 2-4. In some embodiments, the hIFNβ is encoded by a nucleic acid sequence having at least 99% sequence identity to the nucleic acid sequence of any one of SEQ ID NOs: 2-4. In some embodiments, the hIFNβ is encoded by a nucleic acid sequence having the nucleic acid sequence of any one of SEQ ID NOs: 2-4.

In some embodiments, the human interferon alpha 1 (hIFNα1) has the sequence represented by UniProt/SwissProt Database Entry No. P01562 (SEQ ID NO: 5). In some embodiments, the hIFNα is encoded by the nucleic acid sequence of any one of SEQ ID NOs: 6-8. In some embodiments, the hIFNα is encoded by a nucleic acid sequence having at least 70% (e.g., 75%, 80%, 90%, 95%, 96%, 97%, 98%, or 99%) sequence identity to the nucleic acid sequence of any one of SEQ ID NOs: 6-8. In some embodiments, the hIFNα is encoded by a nucleic acid sequence having at least 75% sequence identity to the nucleic acid sequence of any one of SEQ ID NOs: 6-8. In some embodiments, the hIFNα is encoded by a nucleic acid sequence having at least 80% sequence identity to the nucleic acid sequence of any one of SEQ ID NOs: 6-8. In some embodiments, the hIFNα is encoded by a nucleic acid sequence having at least 90% sequence identity to the nucleic acid sequence of any one of SEQ ID NOs: 6-8. In some embodiments, the hIFNα is encoded by a nucleic acid sequence having at least 95% sequence identity to the nucleic acid sequence of any one of SEQ ID NOs: 6-8. In some embodiments, the hIFNα is encoded by a nucleic acid sequence having at least 96% sequence identity to the nucleic acid sequence of any one of SEQ ID NOs: 6-8. In some embodiments, the hIFNα is encoded by a nucleic acid sequence having at least 97% sequence identity to the nucleic acid sequence of any one of SEQ ID NOs: 6-8. In some embodiments, the hIFNα is encoded by a nucleic acid sequence having at least 98% sequence identity to the nucleic acid sequence of any one of SEQ ID NOs: 6-8. In some embodiments, the hIFNα is encoded by a nucleic acid sequence having at least 99% sequence identity to the nucleic acid sequence of any one of SEQ ID NOs: 6-8. In some embodiments, the hIFNα is encoded by a nucleic acid sequence having the nucleic acid sequence of any one of SEQ ID NOs: 6-8.

In some embodiments, the human interferon gamma (hIFNγ) has the sequence represented by UniProt/SwissProt Database Entry No. P01579 (SEQ ID NO: 9). In some embodiments, the hIFNγ is encoded by the nucleic acid sequence of any one of SEQ ID NOs: 10-12. In some embodiments, the hIFNγ is encoded by a nucleic acid sequence having at least 70% (e.g., 75%, 80%, 90%, 95%, 96%, 97%, 98%, or 99%) sequence identity to the nucleic acid sequence of any one of SEQ ID NOs: 10-12. In some embodiments, the hIFNγ is encoded by a nucleic acid sequence having at least 75% sequence identity to the nucleic acid sequence of any one of SEQ ID NOs: 10-12. In some embodiments, the hIFNγ is encoded by a nucleic acid sequence having at least 80% sequence identity to the nucleic acid sequence of any one of SEQ ID NOs: 10-12. In some embodiments, the hIFNγ is encoded by a nucleic acid sequence having at least 90% sequence identity to the nucleic acid sequence of any one of SEQ ID NOs: 10-12. In some embodiments, the hIFNγ is encoded by a nucleic acid sequence having at least 95% sequence identity to the nucleic acid sequence of any one of SEQ ID NOs: 10-12. In some embodiments, the hIFNγ is encoded by a nucleic acid sequence having at least 96% sequence identity to the nucleic acid sequence of any one of SEQ ID NOs: 10-12. In some embodiments, the hIFNγ is encoded by a nucleic acid sequence having at least 97% sequence identity to the nucleic acid sequence of any one of SEQ ID NOs: 10-12. In some embodiments, the hIFNγ is encoded by a nucleic acid sequence having at least 98% sequence identity to the nucleic acid sequence of any one of SEQ ID NOs: 10-12. In some embodiments, the hIFNγ is encoded by a nucleic acid sequence having at least 99% sequence identity to the nucleic acid sequence of any one of SEQ ID NOs: 10-12. In some embodiments, the hIFNγ is encoded by a nucleic acid sequence having the nucleic acid sequence of any one of SEQ ID NOs: 10-12.

In some embodiments, the interferon is mouse interferon beta (mIFNβ). In some embodiments, the mouse interferon beta (mIFNβ) has the sequence represented by UniProt/SwissProt Database Entry No. P01575 (SEQ ID NO: 13). In some embodiments, the mIFNβ is encoded by the nucleic acid sequence of SEQ ID NO: 14. In some embodiments, the mIFNβ is encoded by a nucleic acid sequence having at least 70% (e.g., 75%, 80%, 90%, 95%, 96%, 97%, 98%, or 99%) sequence identity to the nucleic acid sequence of SEQ ID NO: 14. In some embodiments, the mIFNβ is encoded by a nucleic acid sequence having at least 75% sequence identity to the nucleic acid sequence of SEQ ID NO: 14. In some embodiments, the mIFNβ is encoded by a nucleic acid sequence having at least 80% sequence identity to the nucleic acid sequence of SEQ ID NO: 14. In some embodiments, the mIFNβ is encoded by a nucleic acid sequence having at least 90% sequence identity to the nucleic acid sequence of SEQ ID NO: 14. In some embodiments, the mIFNβ is encoded by a nucleic acid sequence having at least 95% sequence identity to the nucleic acid sequence of SEQ ID NO: 14. In some embodiments, the mIFNβ is encoded by a nucleic acid sequence having at least 96% sequence identity to the nucleic acid sequence of SEQ ID NO: 14. In some embodiments, the mIFNβ is encoded by a nucleic acid sequence having at least 97% sequence identity to the nucleic acid sequence of SEQ ID NO: 14. In some embodiments, the mIFNβ is encoded by a nucleic acid sequence having at least 98% sequence identity to the nucleic acid sequence of SEQ ID NO: 14. In some embodiments, the mIFNβ is encoded by a nucleic acid sequence having at least 99% sequence identity to the nucleic acid sequence of SEQ ID NO: 14. In some embodiments, the mIFNβ is encoded by a nucleic acid sequence having the nucleic acid sequence of SEQ ID NO: 14.

In some embodiments, the first interferon polypeptide is canine interferon beta (cIFNβ). In some embodiments, the canine interferon beta (cIFNβ) has the sequence represented by UniProt/UniProtKB Database Entry No. B6E116 (SEQ ID NO: 15). In some embodiments, the interferon is canine interferon beta (cIFNβ) and is encoded by the nucleic acid sequence of SEQ ID NO: 16. In some embodiments, the cIFNβ is encoded by a nucleic acid sequence having at least 70% (e.g., 75%, 80%, 90%, 95%, 96%, 97%, 98%, or 99%) sequence identity to the nucleic acid sequence of SEQ ID NO: 16. In some embodiments, the cIFNβ is encoded by a nucleic acid sequence having at least 75% sequence identity to the nucleic acid sequence of SEQ ID NO: 16. In some embodiments, the cIFNβ is encoded by a nucleic acid sequence having at least 80% sequence identity to the nucleic acid sequence of SEQ ID NO: 16. In some embodiments, the cIFNβ is encoded by a nucleic acid sequence having at least 90% sequence identity to the nucleic acid sequence of SEQ ID NO: 16. In some embodiments, the cIFNβ is encoded by a nucleic acid sequence having at least 95% sequence identity to the nucleic acid sequence of SEQ ID NO: 16. In some embodiments, the cIFNβ is encoded by a nucleic acid sequence having at least 96% sequence identity to the nucleic acid sequence of SEQ ID NO: 16. In some embodiments, the cIFNβ is encoded by a nucleic acid sequence having at least 97% sequence identity to the nucleic acid sequence of SEQ ID NO: 16. In some embodiments, the cIFNβ is encoded by a nucleic acid sequence having at least 98% sequence identity to the nucleic acid sequence of SEQ ID NO: 16. In some embodiments, the cIFNβ is encoded by a nucleic acid sequence having at least 99% sequence identity to the nucleic acid sequence of SEQ ID NO: 16. In some embodiments, the cIFNβ is encoded by a nucleic acid sequence having the nucleic acid sequence of SEQ ID NO: 16.

In some embodiments, the nucleic acid is DNA. In some embodiments, the nucleic acid is RNA.

In some embodiments, the nucleic acids described herein comprise one or more polynucleotides encoding one or more interferons. In some embodiments, the multiple polynucleotides encode polypeptides separated by a self-cleaving peptide. For example, the nucleic acid sequence encoding a first interferon polypeptide and the sequence encoding a second interferon polypeptide may be connected by a sequence encoding a first self-cleaving peptide. Optionally a sequence encoding a linker (e.g., Gly-Ser-Gly) is upstream (5′) from the sequence encoding the self-cleaving peptide. In some embodiments, the sequence encoding a second interferon polypeptide and the sequence encoding a third interferon polypeptide are connected by a sequence encoding a second linker peptide and a sequence encoding a second self-cleaving peptide. In some embodiments, the sequence encoding a second self-cleaving peptide is 3′ from the sequence encoding a second linker peptide.

Suitable self-cleaving peptides include a 2A self-cleaving peptide, such as a P2A self-cleaving peptide, a T2A self-cleaving peptide, a F2A self-cleaving peptide, or an E2A self cleaving peptide. See, e.g., Liu et al., Sci Reports 7 (1): 2193 (2017). In some embodiments, the self-cleaving peptide is a P2A self-cleaving peptide and has the amino acid sequence of SEQ ID NO: 17.

In some embodiments, a first self-cleaving peptide and a second self-cleaving peptide are the same. For example, in some embodiments, both are P2A. In some embodiments, a first self-cleaving peptide and a second self-cleaving peptide are not the same. For example, in some embodiments, a first self-cleaving peptide is a P2A self-cleaving peptide and a second self-cleaving peptide is a T2A self-cleaving peptide. In some embodiments, the second self-cleaving peptide is a T2A self-cleaving peptide and has the amino acid sequence of SEQ ID NO: 18.

In some embodiments, the nucleic acid is delivered using a plasmid (e.g., circular DNA molecules that can autonomously replicate inside a cell), cosmid (e.g., pWE or sCos vectors), artificial chromosome, human artificial chromosome (HAC), yeast artificial chromosomes (YAC), bacterial artificial chromosome (BAC), minicircle, doggybone, P1-derived artificial chromosomes (PAC), phagemid, phage derivative, bacmid, or virus. In some embodiments, the nucleic acid is within a vector selected from the list consisting of: pSF-CMV-NEO-NH2-PPT-3XFLAG, pSF-CMV-NEO-COOH-3XFLAG, pSF-CMV-PURO-NH2-GST-TEV, pCEP4 pDEST27, pSF-CMV-Ub-KrYFP, pSF-CMV-FMDV-daGFP, pEFla-mCherry-N1 vector, pEF1a-tdTomato vector, pSF-CMV-FMDV-Hygro, pSF-CMV-PGK-Puro, pMCP-tag(m), and pSF-CMV-PURO-NH2-CMYC, pSF-OXB20-BetaGal, pSF-OXB20-fLuc, pSF-OXB20, pSF-Tac, pSF-OXB20-COOH-TEV-FLAG(R)-6His, pRI 101-AN DNA, pCambia2301, pTYB21, pKLAC2, pAc5.1/V5-His A, and pDEST8.

In some embodiments, the nucleic acid is delivered using a virus. In some embodiments, the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus. In some embodiments, the virus is an alphavirus. In some embodiments, the virus is a parvovirus. In some embodiments, the virus is an adenovirus. In some embodiments, the virus is an AAV. In some embodiments, the virus is a baculovirus. In some embodiments, the virus is a Dengue virus. In some embodiments, the virus is a lentivirus. In some embodiments, the virus is a herpesvirus. In some embodiments, the virus is a poxvirus. In some embodiments, the virus is an anellovirus. In some embodiments, the virus is a bocavirus. In some embodiments, the virus is a vaccinia virus. In some embodiments, the virus is a retrovirus.

In some embodiments, the virus is an AAV. In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof. In some embodiments, the herpesvirus is HSV type 1, HSV-2, VZV, EBV, CMV, HHV-6, HHV-7, or HHV-8.

In some embodiments, the virus is AAV1 or a derivative thereof. In some embodiments, the virus is AAV2 or a derivative thereof. In some embodiments, the virus is AAV3 or a derivative thereof. In some embodiments, the virus is AAV4 or a derivative thereof. In some embodiments, the virus is AAV5 or a derivative thereof. In some embodiments, the virus is AAV6 or a derivative thereof. In some embodiments, the virus is AAV7 or a derivative thereof. In some embodiments, the virus is AAV8 or a derivative thereof. In some embodiments, the virus is AAV9 or a derivative thereof. In some embodiments, the virus is AAV10 or a derivative thereof. In some embodiments, the virus is AAV11 or a derivative thereof. In some embodiments, the virus is AAV12 or a derivative thereof. In some embodiments, the virus is AAV13 or a derivative thereof. In some embodiments, the virus is AAV14 or a derivative thereof. In some embodiments, the virus is AAV15 or a derivative thereof. In some embodiments, the virus is AAV16 or a derivative thereof. In some embodiments, the virus is AAV-rh8 or a derivative thereof. In some embodiments, the virus is AAV-rh10 or a derivative thereof. In some embodiments, the virus is AAV-rh20 or a derivative thereof. In some embodiments, the virus is AAV-rh39 or a derivative thereof. In some embodiments, the virus is AAV-rh74 or a derivative thereof. In some embodiments, the virus is AAV-rhM4-1 or a derivative thereof. In some embodiments, the virus is AAV-hu37 or a derivative thereof. In some embodiments, the virus is AAV-Anc80 or a derivative thereof. In some embodiments, the virus is AAV-Anc80L65 or a derivative thereof. In some embodiments, the virus is AAV-7m8 or a derivative thereof. In some embodiments, the virus is AAV-PHP-B or a derivative thereof. In some embodiments, the virus is AAV-PHP-EB or a derivative thereof. In some embodiments, the virus is AAV-2.5 or a derivative thereof. In some embodiments, the virus is AAV-2tYF or a derivative thereof. In some embodiments, the virus is AAV-3B or a derivative thereof. In some embodiments, the virus is AAV-LK03 or a derivative thereof. In some embodiments, the virus is AAV-HSC1 or a derivative thereof. In some embodiments, the virus is AAV-HSC2 or a derivative thereof. In some embodiments, the virus is AAV-HSC3 or a derivative thereof. In some embodiments, the virus is AAV-HSC4 or a derivative thereof. In some embodiments, the virus is AAV-HSC5 or a derivative thereof. In some embodiments, the virus is AAV-HSC6 or a derivative thereof. In some embodiments, the virus is AAV-HSC7 or a derivative thereof. In some embodiments, the virus is AAV-HSC8 or a derivative thereof. In some embodiments, the virus is AAV-HSC9 or a derivative thereof. In some embodiments, the virus is AAV-HSC10 or a derivative thereof. In some embodiments, the virus is AAV-HSC11 or a derivative thereof. In some embodiments, the virus is AAV-HSC12 or a derivative thereof. In some embodiments, the virus is AAV-HSC13 or a derivative thereof. In some embodiments, the virus is AAV-HSC14 or a derivative thereof. In some embodiments, the virus is AAV-HSC15 or a derivative thereof. In some embodiments, the virus is AAV-TT or a derivative thereof. In some embodiments, the virus is AAV-DJ/8 or a derivative thereof. In some embodiments, the virus is AAV-Myo or a derivative thereof. In some embodiments, the virus is AAV-NP40 or a derivative thereof. In some embodiments, the virus is AAV-NP59 or a derivative thereof. In some embodiments, the virus is AAV-NP22 or a derivative thereof. In some embodiments, the virus is AAV-NP66 or a derivative thereof. In some embodiments, the virus is AAV-HSC16 or a derivative thereof.

In some embodiments, the virus is HSV-1 or a derivative thereof. In some embodiments, the virus is HSV-2 or a derivative thereof. In some embodiments, the virus is VZV or a derivative thereof. In some embodiments, the virus is EBV or a derivative thereof. In some embodiments, the virus is CMV or a derivative thereof. In some embodiments, the virus is HHV-6 or a derivative thereof. In some embodiments, the virus is HHV-7 or a derivative thereof. In some embodiments, the virus is HHV-8 or a derivative thereof.

In some embodiments, the nucleic acid comprises a promoter, an enhancer, an intron, a microRNA, a linker, a splicing element, a regulatory element, a polyA signal sequence, or combinations thereof. In some embodiments, the regulatory element is Woodchuck Hepatitis Virus (WHP) Posttranscriptional Regulatory Element (WPRE). In some embodiments, the nucleic acid comprises a promoter. In some embodiments, the nucleic acid comprises an enhancer. In some embodiments, the nucleic acid comprises an intron. In some embodiments, the nucleic acid comprises a microRNA. In some embodiments, the nucleic acid comprises a linker. In some embodiments, the nucleic acid comprises a splicing element. In some embodiments, the nucleic acid comprises a polyA signal sequence.

In some embodiments, the polyA signal sequence is from SV40. In some embodiments, the polyA signal sequence comprises the nucleic acid sequence of SEQ ID NO: 19. In some embodiments, the polyA signal sequence is from bovine growth hormone (bGH). Examples of other suitable polyA signals include, a synthetic polyA signal, a polyA from human growth hormone (hGH), rabbit beta-globin (RGB), or modified RGB (mRGB).

In some embodiments, the nucleic acid comprises a promoter. In some embodiments, the promoter is selected from the group consisting of a mini promoter, an inducible promoter, a constitutive promoter, and derivatives thereof. In some embodiments, the promoter is selected from the group consisting of CMV, CBA, EF1a, CAG, PGK, TRE, U6, UAS, T7, Sp6, lac, araBad, trp, Ptac, p5, p19, p40, Synapsin, CaMKII, GRK1, and derivatives thereof. In some embodiments, the promoter is the CMV promoter or a derivative thereof. In some embodiments, the promoter is the CBA promoter or a derivative thereof. In some embodiments, the promoter is the EF1a promoter or a derivative thereof. In some embodiments, the promoter is the CAG promoter or a derivative thereof. In some embodiments, the promoter is the PGK promoter or a derivative thereof. In some embodiments, the promoter is the TRE promoter or a derivative thereof. In some embodiments, the promoter is the U6 promoter or a derivative thereof. In some embodiments, the promoter is the UAS promoter or a derivative thereof. In some embodiments, the promoter is the T7 promoter or a derivative thereof. In some embodiments, the promoter is the Sp6 promoter or a derivative thereof. In some embodiments, the promoter is the lac promoter or a derivative thereof. In some embodiments, the promoter is the araBad promoter or a derivative thereof. In some embodiments, the promoter is the trp promoter or a derivative thereof. In some embodiments, the promoter is the Ptac promoter or a derivative thereof. In some embodiments, the promoter is the p5 promoter or a derivative thereof. In some embodiments, the promoter is the p19 promoter or a derivative thereof. In some embodiments, the promoter is the p40 promoter or a derivative thereof. In some embodiments, the promoter is the Synapsin promoter or a derivative thereof. In some embodiments, the promoter is the CaMKII promoter or a derivative thereof. In some embodiments, the promoter is the GRK1 promoter or a derivative thereof. In some embodiments, the promoter is a mini promoter or a derivative thereof. In some embodiments, the promoter is an inducible promoter.

In some embodiments, the nucleic acid comprises a backbone polynucleotide. In some embodiments, the backbone polynucleotide comprises an antibiotic resistance gene, an origin of replication, an open reading frame, or combinations thereof. In some embodiments, the backbone polynucleotide comprises an antibiotic resistance gene, an origin of replication, and an open reading frame.

In some embodiments, the backbone polynucleotide comprises an antibiotic resistance gene. In some embodiments, the antibiotic resistance gene encodes an aminoglycoside, a beta-lactam, a macrolide, a tetracycline, or a derivative thereof. In some embodiments, the backbone polynucleotide comprises an aminoglycoside or a derivative thereof. In some embodiments, the backbone polynucleotide comprises a beta-lactam or a derivative thereof. In some embodiments, the backbone polynucleotide comprises a macrolide or a derivative thereof. In some embodiments, the backbone polynucleotide comprises a tetracycline or a derivative thereof.

In some embodiments, the antibiotic resistance gene encodes a kanamycin, spectinomycin, streptomycin, ampicillin, carbenicillin, bleomycin, erythromycin, polymyxin B, tetracycline, chloramphenicol, neomycin, or zeocin resistance gene, or a derivative thereof. In some embodiments, the antibiotic resistance gene encodes a kanamycin resistance gene.

In some embodiments, the kanamycin resistance gene comprises a nucleic acid sequence having at least 70% (e.g., 75%, 80%, 90%, 95%, 96%, 97%, 98%, or 99%) sequence identity to the nucleic acid sequence of any one of SEQ ID NOs: 20 or 21. In some embodiments, the kanamycin resistance gene comprises a nucleic acid sequence having at least 75% sequence identity to the nucleic acid sequence of any one of SEQ ID NOs: 20 or 21. In some embodiments, the kanamycin resistance gene comprises a nucleic acid sequence having at least 80% sequence identity to the nucleic acid sequence of any one of SEQ ID NOs: 20 or 21. In some embodiments, the kanamycin resistance gene comprises a nucleic acid sequence having at least 90% sequence identity to the nucleic acid sequence of any one of SEQ ID NOs: 20 or 21. In some embodiments, the kanamycin resistance gene comprises a nucleic acid sequence having at least 95% sequence identity to the nucleic acid sequence of any one of SEQ ID NOs: 20 or 21. In some embodiments, the kanamycin resistance gene comprises a nucleic acid sequence having at least 96% sequence identity to the nucleic acid sequence of any one of SEQ ID NOs: 20 or 21. In some embodiments, the kanamycin resistance gene comprises a nucleic acid sequence having at least 97% sequence identity to the nucleic acid sequence of any one of SEQ ID NOs: 20 or 21. In some embodiments, the kanamycin resistance gene comprises a nucleic acid sequence having at least 98% sequence identity to the nucleic acid sequence of any one of SEQ ID NOs: 20 or 21. In some embodiments, the kanamycin resistance gene comprises a nucleic acid sequence having at least 99% sequence identity to the nucleic acid sequence of any one of SEQ ID NOs: 20 or 21. In some embodiments, the kanamycin resistance gene comprises a nucleic acid sequence having the nucleic acid sequence of any one of SEQ ID NOs: 20 or 21.

In some embodiments, the ampicillin resistance gene comprises a nucleic acid sequence having at least 70% (e.g., 75%, 80%, 90%, 95%, 96%, 97%, 98%, or 99%) sequence identity to the nucleic acid sequence of SEQ ID NO: 34. In some embodiments, the ampicillin resistance gene comprises a nucleic acid sequence having at least 75% sequence identity to the nucleic acid sequence of SEQ ID NO: 34. In some embodiments, the ampicillin resistance gene comprises a nucleic acid sequence having at least 80% sequence identity to the nucleic acid sequence of SEQ ID NO: 34. In some embodiments, the ampicillin resistance gene comprises a nucleic acid sequence having at least 90% sequence identity to the nucleic acid sequence of SEQ ID NO: 34. In some embodiments, the ampicillin resistance gene comprises a nucleic acid sequence having at least 95% sequence identity to the nucleic acid sequence of SEQ ID NO: 34. In some embodiments, the ampicillin resistance gene comprises a nucleic acid sequence having at least 96% sequence identity to the nucleic acid sequence of SEQ ID NO: 34. In some embodiments, the ampicillin resistance gene comprises a nucleic acid sequence having at least 97% sequence identity to the nucleic acid sequence of SEQ ID NO: 34. In some embodiments, the ampicillin resistance gene comprises a nucleic acid sequence having at least 98% sequence identity to the nucleic acid sequence of SEQ ID NO: 34. In some embodiments, the ampicillin resistance gene comprises a nucleic acid sequence having at least 99% sequence identity to the nucleic acid sequence of SEQ ID NO: 34. In some embodiments, the ampicillin resistance gene comprises a nucleic acid sequence having the nucleic acid sequence of SEQ ID NO: 34.

In some embodiments, the backbone polynucleotide comprises an origin of replication. In some embodiments, the origin of replication is pMB1, pBR322, ColE1, R6K, p15A, pSC101, ColE2, F1, pUC, or combinations or a derivative thereof. In some embodiments, the origin of replication is pMB1 or a derivative thereof. In some embodiments, the origin of replication is pBR322 or a derivative thereof. In some embodiments, the origin of replication is ColE1 or a derivative thereof. In some embodiments, the origin of replication is R6K or a derivative thereof. In some embodiments, the origin of replication is p15A or a derivative thereof. In some embodiments, the origin of replication is pSC101 or a derivative thereof. In some embodiments, the origin of replication is ColE2 or a derivative thereof. In some embodiments, the origin of replication is F1 or a derivative thereof. In some embodiments, the origin of replication is pUC or a derivative thereof.

In some embodiments, nucleic acids of the disclosure comprises a woodchuck hepatitis virus post-transcriptional element or a derivative thereof.

Nucleic acids described herein, in some embodiments, comprise a reporter sequence for co-expression, such as but not limited to lacZ, eGFP, GFP, degradable GFP (d2eGFP), CFP, YFP, RFP, BFP, mCherry, mCardinal, Firefly luciferase (fLuc), Renilla luciferase, NanoLuc luciferase (nLuc), and tdTomato. In some embodiments, the vector comprises a selectable marker.

In some embodiments, the reporter is lacZ. In some embodiments, the reporter is eGFP. In some embodiments, the reporter is GFP. In some embodiments, the reporter is d2eGFP. In some embodiments, the reporter is CFP. In some embodiments, the reporter is YFP. In some embodiments, the reporter is RFP. In some embodiments, the reporter is BFP. In some embodiments, the reporter is mCherry. In some embodiments, the reporter is mCardinal. In some embodiments, the reporter is fLuc. In some embodiments, the reporter is Renilla luciferase. In some embodiments, the reporter is nLuc. In some embodiments, the reporter is tdTomato.

In some embodiments, the reporter has been modified such that the reporter has an ATG or a potential alternative in-frame start codon removed, while the protein sequence of the reporter is unchanged.

In some embodiments, the eGFP comprises a nucleic acid sequence having at least 70% (e.g., 75%, 80%, 90%, 95%, 96%, 97%, 98%, or 99%) sequence identity to the nucleic acid sequence of any one of SEQ ID NOs: 35 or 36. In some embodiments, the eGFP comprises a nucleic acid sequence having at least 75% sequence identity to the nucleic acid sequence of any one of SEQ ID NOs: 35 or 36. In some embodiments, the eGFP comprises a nucleic acid sequence having at least 80% sequence identity to the nucleic acid sequence of any one of SEQ ID NOs: 35 or 36. In some embodiments, the eGFP comprises a nucleic acid sequence having at least 90% sequence identity to the nucleic acid sequence of any one of SEQ ID NOs: 35 or 36. In some embodiments, the eGFP comprises a nucleic acid sequence having at least 95% sequence identity to the nucleic acid sequence of any one of SEQ ID NOs: 35 or 36. In some embodiments, the eGFP comprises a nucleic acid sequence having at least 96% sequence identity to the nucleic acid sequence of any one of SEQ ID NOs: 35 or 36. In some embodiments, the eGFP comprises a nucleic acid sequence having at least 97% sequence identity to the nucleic acid sequence of any one of SEQ ID NOs: 35 or 36. In some embodiments, the eGFP comprises a nucleic acid sequence having at least 98% sequence identity to the nucleic acid sequence of any one of SEQ ID NOs: 35 or 36. In some embodiments, the eGFP comprises a nucleic acid sequence having at least 99% sequence identity to the nucleic acid sequence of any one of SEQ ID NOs: 35 or 36. In some embodiments, the eGFP comprises a nucleic acid sequence having the nucleic acid sequence of any one of SEQ ID NOs: 35 or 36.

In some embodiments, the eGFP has been modified such that the eGFP has an ATG or a potential alternative in-frame start codon removed, while the protein sequence of the eGFP is unchanged.

In some embodiments, the mCardinal comprises a nucleic acid sequence having at least 70% (e.g., 75%, 80%, 90%, 95%, 96%, 97%, 98%, or 99%) sequence identity to the nucleic acid sequence of any one of SEQ ID NOs: 37 or 38. In some embodiments, the mCardinal comprises a nucleic acid sequence having at least 75% sequence identity to the nucleic acid sequence of any one of SEQ ID NOs: 37 or 38. In some embodiments, the mCardinal comprises a nucleic acid sequence having at least 80% sequence identity to the nucleic acid sequence of any one of SEQ ID NOs: 37 or 38. In some embodiments, the mCardinal comprises a nucleic acid sequence having at least 90% sequence identity to the nucleic acid sequence of any one of SEQ ID NOs: 37 or 38. In some embodiments, the mCardinal comprises a nucleic acid sequence having at least 95% sequence identity to the nucleic acid sequence of any one of SEQ ID NOs: 37 or 38. In some embodiments, the mCardinal comprises a nucleic acid sequence having at least 96% sequence identity to the nucleic acid sequence of any one of SEQ ID NOs: 37 or 38. In some embodiments, the mCardinal comprises a nucleic acid sequence having at least 97% sequence identity to the nucleic acid sequence of any one of SEQ ID NOs: 37 or 38. In some embodiments, the mCardinal comprises a nucleic acid sequence having at least 98% sequence identity to the nucleic acid sequence of any one of SEQ ID NOs: 37 or 38. In some embodiments, the mCardinal comprises a nucleic acid sequence having at least 99% sequence identity to the nucleic acid sequence of any one of SEQ ID NOs: 37 or 38. In some embodiments, the mCardinal comprises a nucleic acid sequence having the nucleic acid sequence of any one of SEQ ID NOs: 37 or 38.

In some embodiments, the mCardinal has been modified such that the mCardinal has an ATG or a potential alternative in-frame start codon removed, while the protein sequence of the mCardinal is unchanged.

In some embodiments, the fLuc comprises a nucleic acid sequence having at least 70% (e.g., 75%, 80%, 90%, 95%, 96%, 97%, 98%, or 99%) sequence identity to the nucleic acid sequence of any one of SEQ ID NOs: 39-41. In some embodiments, the fLuc comprises a nucleic acid sequence having at least 75% sequence identity to the nucleic acid sequence of any one of SEQ ID NOs: 39-41. In some embodiments, the fLuc comprises a nucleic acid sequence having at least 80% sequence identity to the nucleic acid sequence of any one of SEQ ID NOs: 39-41. In some embodiments, the fLuc comprises a nucleic acid sequence having at least 90% sequence identity to the nucleic acid sequence of any one of SEQ ID NOs: 39-41. In some embodiments, the fLuc comprises a nucleic acid sequence having at least 95% sequence identity to the nucleic acid sequence of any one of SEQ ID NOs: 39-41. In some embodiments, the fLuc comprises a nucleic acid sequence having at least 96% sequence identity to the nucleic acid sequence of any one of SEQ ID NOs: 39-41. In some embodiments, the fLuc comprises a nucleic acid sequence having at least 97% sequence identity to the nucleic acid sequence of any one of SEQ ID NOs: 39-41. In some embodiments, the fLuc comprises a nucleic acid sequence having at least 98% sequence identity to the nucleic acid sequence of any one of SEQ ID NOs: 39-41. In some embodiments, the fLuc comprises a nucleic acid sequence having at least 99% sequence identity to the nucleic acid sequence of any one of SEQ ID NOs: 39-41. In some embodiments, the fLuc comprises a nucleic acid sequence having the nucleic acid sequence of any one of SEQ ID NOs: 39-41.

In some embodiments, the fLuc has been modified such that the fLuc has an ATG or a potential alternative in-frame start codon removed, while the protein sequence of the fLuc is unchanged.

In some embodiments, the nLuc comprises a nucleic acid sequence having at least 70% (e.g., 75%, 80%, 90%, 95%, 96%, 97%, 98%, or 99%) sequence identity to the nucleic acid sequence of SEQ ID NO: 42. In some embodiments, the nLuc comprises a nucleic acid sequence having at least 75% sequence identity to the nucleic acid sequence of SEQ ID NO: 42. In some embodiments, the nLuc comprises a nucleic acid sequence having at least 80% sequence identity to the nucleic acid sequence of SEQ ID NO: 42. In some embodiments, the nLuc comprises a nucleic acid sequence having at least 90% sequence identity to the nucleic acid sequence of SEQ ID NO: 42. In some embodiments, the nLuc comprises a nucleic acid sequence having at least 95% sequence identity to the nucleic acid sequence of SEQ ID NO: 42. In some embodiments, the nLuc comprises a nucleic acid sequence having at least 96% sequence identity to the nucleic acid sequence of SEQ ID NO: 42. In some embodiments, the nLuc comprises a nucleic acid sequence having at least 97% sequence identity to the nucleic acid sequence of SEQ ID NO: 42. In some embodiments, the nLuc comprises a nucleic acid sequence having at least 98% sequence identity to the nucleic acid sequence of SEQ ID NO: 42. In some embodiments, the nLuc comprises a nucleic acid sequence having at least 99% sequence identity to the nucleic acid sequence of SEQ ID NO: 42. In some embodiments, the nLuc comprises a nucleic acid sequence having the nucleic acid sequence of SEQ ID NO: 42.

In some embodiments, the nLuc has been modified such that the nLuc has an ATG or a potential alternative in-frame start codon removed, while the protein sequence of the nLuc is unchanged.

In some embodiments, the d2eGFP comprises a nucleic acid sequence having at least 70% (e.g., 75%, 80%, 90%, 95%, 96%, 97%, 98%, or 99%) sequence identity to the nucleic acid sequence of SEQ ID NO: 43. In some embodiments, the d2eGFP comprises a nucleic acid sequence having at least 75% sequence identity to the nucleic acid sequence of SEQ ID NO: 43. In some embodiments, the d2eGFP comprises a nucleic acid sequence having at least 80% sequence identity to the nucleic acid sequence of SEQ ID NO: 43. In some embodiments, the d2eGFP comprises a nucleic acid sequence having at least 90% sequence identity to the nucleic acid sequence of SEQ ID NO: 43. In some embodiments, the d2eGFP comprises a nucleic acid sequence having at least 95% sequence identity to the nucleic acid sequence of SEQ ID NO: 43. In some embodiments, the d2eGFP comprises a nucleic acid sequence having at least 96% sequence identity to the nucleic acid sequence of SEQ ID NO: 43. In some embodiments, the d2eGFP comprises a nucleic acid sequence having at least 97% sequence identity to the nucleic acid sequence of SEQ ID NO: 43. In some embodiments, the d2eGFP comprises a nucleic acid sequence having at least 98% sequence identity to the nucleic acid sequence of SEQ ID NO: 43. In some embodiments, the d2eGFP comprises a nucleic acid sequence having at least 99% sequence identity to the nucleic acid sequence of SEQ ID NO: 43. In some embodiments, the d2eGFP comprises a nucleic acid sequence having the nucleic acid sequence of SEQ ID NO: 43.

In some embodiments, the deGFP has been modified such that the d2eGFP has an ATG or a potential alternative in-frame start codon removed, while the protein sequence of the d2eGFP is unchanged.

### Recombinant Viral Vectors

Viral genomes provide a rich source of vectors that are used for the efficient delivery of exogenous polynucleotides into a cell. In some embodiments, the viral vector is derived from an anellovirus, parvovirus (e.g., AAV and bocavirus), retrovirus (e.g., Retroviridae family viral vector), adenovirus (e.g., Ad5, Ad26, Ad34, Ad35, and Ad48), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses comprising adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses comprise Norwalk virus, togavirus, flavivirus, reoviruses, papovavirus, hepadnavirus, human papilloma virus, human foamy virus, and hepatitis virus, for example. Examples of retroviruses are avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus. Other examples are murine leukemia viruses, murine sarcoma viruses, murine mammary tumor virus, bovine leukemia virus, feline leukemia virus, feline sarcoma virus, avian leukemia virus, human T-cell leukemia virus, baboon endogenous virus, Gibbon ape leukemia virus, Pfizer monkey virus, simian immunodeficiency virus, simian sarcoma virus, Rous sarcoma virus, and lentiviruses.

Various recombinant viral vectors are contemplated herein. In some embodiments, the recombinant viral vector is a recombinant herpesvirus, such as Herpes Simplex Virus (HSV), such as HSV type 1 (HSV-1), HSV-2, Varicella Zoster Virus (VZV), Epstein-Barr Virus (EBV), Cytomegalovirus (CMV), human herpes virus (HHV) 6 (HHV-6), HHV-7, or HHV-8. In some embodiments, the HSV is HSV-1. In some embodiments, the HSV is HSV-2. In some embodiments, the HSV is VZV. In some embodiments, the HSV is EBV. In some embodiments, the HSV is CMV. In some embodiments, the HSV is HHV-6. In some embodiments, the HSV is HHV-7. In some embodiments, the HSV is HHV-8.

In some embodiments, the recombinant viral vector is an AAV, such as AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16.

In some embodiments, a recombinant AAV is AAV1 or a derivative thereof. In some embodiments, a recombinant AAV is AAV2 or a derivative thereof. In some embodiments, a recombinant AAV is AAV3 or a derivative thereof. In some embodiments, a recombinant AAV is AAV4 or a derivative thereof. In some embodiments, a recombinant AAV is AAV5 or a derivative thereof. In some embodiments, a recombinant AAV is AAV6 or a derivative thereof. In some embodiments, a recombinant AAV is AAV7 or a derivative thereof. In some embodiments, a recombinant AAV is AAV8 or a derivative thereof. In some embodiments, a recombinant AAV is AAV9 or a derivative thereof. In some embodiments, a recombinant AAV is AAV10 or a derivative thereof. In some embodiments, a recombinant AAV is AAV11 or a derivative thereof. In some embodiments, a recombinant AAV is AAV12 or a derivative thereof. In some embodiments, a recombinant AAV is AAV13 or a derivative thereof. In some embodiments, a recombinant AAV is AAV14 or a derivative thereof. In some embodiments, a recombinant AAV is AAV15 or a derivative thereof. In some embodiments, a recombinant AAV is AAV16 or a derivative thereof. In some embodiments, a recombinant AAV is AAV-rh8 or a derivative thereof. In some embodiments, a recombinant AAV is AAV-rh10 or a derivative thereof. In some embodiments, a recombinant AAV is AAV-rh20 or a derivative thereof. In some embodiments, a recombinant AAV is AAV-rh39 or a derivative thereof. In some embodiments, a recombinant AAV is AAV-rh74 or a derivative thereof. In some embodiments, a recombinant AAV is AAV-rhM4-1 or a derivative thereof. In some embodiments, a recombinant AAV is AAV-hu37 or a derivative thereof. In some embodiments, a recombinant AAV is AAV-Anc80 or a derivative thereof. In some embodiments, a recombinant AAV is AAV-Anc80L65 or a derivative thereof. In some embodiments, a recombinant AAV is AAV-7m8 or a derivative thereof. In some embodiments, a recombinant AAV is AAV-PHP-B or a derivative thereof. In some embodiments, a recombinant AAV is AAV-PHP-EB or a derivative thereof. In some embodiments, a recombinant AAV is AAV-2.5 or a derivative thereof. In some embodiments, a recombinant AAV is AAV-2tYF or a derivative thereof. In some embodiments, a recombinant AAV is AAV-3B or a derivative thereof. In some embodiments, a recombinant AAV is AAV-LK03 or a derivative thereof. In some embodiments, a recombinant AAV is AAV-HSC1 or a derivative thereof. In some embodiments, a recombinant AAV is AAV-HSC2 or a derivative thereof. In some embodiments, a recombinant AAV is AAV-HSC3 or a derivative thereof. In some embodiments, a recombinant AAV is AAV-HSC4 or a derivative thereof. In some embodiments, a recombinant AAV is AAV-HSC5 or a derivative thereof. In some embodiments, a recombinant AAV is AAV-HSC6 or a derivative thereof. In some embodiments, a recombinant AAV is AAV-HSC7 or a derivative thereof. In some embodiments, a recombinant AAV is AAV-HSC8 or a derivative thereof. In some embodiments, a recombinant AAV is AAV-HSC9 or a derivative thereof. In some embodiments, a recombinant AAV is AAV-HSC10 or a derivative thereof. In some embodiments, a recombinant AAV is AAV-HSC11 or a derivative thereof. In some embodiments, a recombinant AAV is AAV-HSC12 or a derivative thereof. In some embodiments, a recombinant AAV is AAV-HSC13 or a derivative thereof. In some embodiments, a recombinant AAV is AAV-HSC14 or a derivative thereof. In some embodiments, a recombinant AAV is AAV-HSC15 or a derivative thereof. In some embodiments, a recombinant AAV is AAV-TT or a derivative thereof. In some embodiments, a recombinant AAV is AAV-DJ/8 or a derivative thereof. In some embodiments, a recombinant AAV is AAV-Myo or a derivative thereof. In some embodiments, a recombinant AAV is AAV-NP40 or a derivative thereof. In some embodiments, a recombinant AAV is AAV-NP59 or a derivative thereof. In some embodiments, a recombinant AAV is AAV-NP22 or a derivative thereof. In some embodiments, a recombinant AAV is AAV-NP66 or a derivative thereof. In some embodiments, a recombinant AAV is AAV-HSC16 or a derivative thereof.

### Recombinant AAV Vectors

Recombinant adeno-associated viral vectors (rAAV) can be used for incorporation of genes to facilitate their introduction into a cell, such as a target cell.

Described herein, in certain embodiments, are viral vectors, comprising from 5′ to 3′: (a) a 5′ AAV inverted terminal repeat (ITR); (b) a CAG promoter; (c) a first polynucleotide comprising a minigene, the minigene comprising a splice modulator binding site; (d) a second polynucleotide encoding an interferon; and (e) a 3′ AAV ITR.

Described herein, in certain embodiments, are viral vectors, comprising from 5′ to 3′: (a) a 5′ AAV inverted terminal repeat (ITR); (b) a CAG promoter comprising a nucleic acid sequence having at least 80% sequence identity to the nucleic acid sequence of any one of SEQ ID NOs: 22, 23, 44, or 45; (c) a first polynucleotide comprising a minigene, the minigene comprising a splice modulator binding site and at least about 80% sequence identity to the nucleic acid sequence of SEQ ID NO: 32 or SEQ ID NO: 33; (d) a second polynucleotide encoding an interferon, the second polynucleotide comprising at least about 80% sequence identity to the nucleic acid sequence of SEQ ID NO: 4; and (e) a 3′ AAV ITR.

Described herein are rAAV vectors comprising nucleic acids encoding one or more interferons described herein. In some embodiments, the rAAV vector comprises a nucleic acid that encodes a single interferon polypeptide. In some embodiments, the rAAV vector comprises a bicistronic or tricistronic nucleic acid that encodes multiple interferon polypeptides. In some embodiments, the nucleic acids described herein encode one or more interferons to be delivered to a cell or tissue, as well as regulatory elements controlling expression of the one or more interferons. Regulatory elements include, but are not limited to, promoters, enhancers, terminator sequences, polyadenylation sequences, mRNA stability sequences (e.g. Woodchuck Hepatitis Virus Posttranscriptional Regulatory Element; WPRE), sequences that allow for internal ribosome entry sites (IRES) of bicistronic mRNA, sequences necessary for episome maintenance (e.g., ITRs), sequences that avoid or inhibit viral recognition by Toll-like or RIG-like receptors (e.g. TLR-7, -8, -9, MDA-5, RIG-1 and/or DAI), and/or sequences necessary for transduction into cells.

In some embodiments, the nucleic acids described herein comprise a CAG promoter operably linked to the polynucleotides encoding one or more interferon polypeptides. In some embodiments, the CAG promoter comprises a first segment comprising a cytomegalovirus (CMV) enhancer sequence, a second segment comprising a chicken beta-actin (CBA) gene promoter element, a third segment comprising a spacer sequence, and a fourth segment comprising a rabbit beta-globin splice acceptor. In some embodiments, the order of the segments 5′-to-3′ is first, second, third, and fourth.

In some embodiments, the CAG promoter comprises the nucleic acid sequence of any one of SEQ ID NOs: 22, 23, 44, or 45. In some embodiments, the CAG promoter comprises a nucleic acid sequence having at least 70% (e.g., 75%, 80%, 90%, 95%, 96%, 97%, 98%, or 99%) sequence identity to the nucleic acid sequence of any one of SEQ ID NOs: 22, 23, 44, or 45. In some embodiments, the CAG promoter comprises a nucleic acid sequence having at least 75% sequence identity to the nucleic acid sequence of any one of SEQ ID NOs: 22, 23, 44, or 45. In some embodiments, the CAG promoter comprises a nucleic acid sequence having at least 80% sequence identity to the nucleic acid sequence of any one of SEQ ID NOs: 22, 23, 44, or 45. In some embodiments, the CAG promoter comprises a nucleic acid sequence having at least 90% sequence identity to the nucleic acid sequence of any one of SEQ ID NOs: 22, 23, 44, or 45. In some embodiments, the CAG promoter comprises a nucleic acid sequence having at least 95% sequence identity to the nucleic acid sequence of any one of SEQ ID NOs: 22, 23, 44, or 45. In some embodiments, the CAG promoter comprises a nucleic acid sequence having at least 96% sequence identity to the nucleic acid sequence of any one of SEQ ID NOs: 22, 23, 44, or 45. In some embodiments, the CAG promoter comprises a nucleic acid sequence having at least 97% sequence identity to the nucleic acid sequence of any one of SEQ ID NOs: 22, 23, 44, or 45. In some embodiments, the CAG promoter comprises a nucleic acid sequence having at least 98% sequence identity to the nucleic acid sequence of any one of SEQ ID NOs: 22, 23, 44, or 45. In some embodiments, the CAG promoter comprises a nucleic acid sequence having at least 99% sequence identity to the nucleic acid sequence of any one of SEQ ID NOs: 22, 23, 44, or 45. In some embodiments, the CAG promoter comprises a nucleic acid sequence having the nucleic acid sequence of any one of SEQ ID NOs: 22, 23, 44, or 45. In some embodiments, the CAG promoter comprises contiguous nucleotides of about 20, about 25, about 40, about 50, about 75, about 100, about 125, about 150, about 175, about 180, about 200, about 220, about 240, about 260, about 280, about 300, about 320, about 340, about 360, about 380, about 400, or about 440 nucleotides of any one of SEQ ID NOs: 22, 23, 44, or 45. In some embodiments, the CAG promoter comprises a nucleic acid sequence having at least 70% (e.g., 75%, 80%, 90%, 95%, 96%, 97%, 98%, or 99%) sequence identity to contiguous nucleotides of about 20, about 25, about 40, about 50, about 75, about 100, about 125, about 150, about 175, about 180, about 200, about 220, about 240, about 260, about 280, about 300, about 320, about 340, about 360, about 380, about 400, or about 440 nucleotides of any one of SEQ ID NOs: 22, 23, 44, or 45.

In some embodiments, the CMV enhancer is derived from human CMV. In some embodiments, the CMV enhancer comprises various repeated sequence elements as described in Boshart et al. Cell 41 (2): 521-530 (1985), and in U.S. Pat. Nos. 5,168,062 and 5,385,839, each of which is incorporated herein by reference. In some embodiments, the CMV enhancer comprises the nucleic acid sequence of any one of SEQ ID NOs: 25-27. In some embodiments, the CMV enhancer comprises a nucleic acid sequence having at least 70% (e.g., 75%, 80%, 90%, 95%, 96%, 97%, 98%, or 99%) sequence identity to the nucleic acid sequence of any one of SEQ ID NOs: 25-27. In some embodiments, the CMV enhancer comprises a nucleic acid sequence having at least 75% sequence identity to the nucleic acid sequence of any one of SEQ ID NOs: 25-27. In some embodiments, the CMV enhancer comprises a nucleic acid sequence having at least 80% sequence identity to the nucleic acid sequence of any one of SEQ ID NOs: 25-27. In some embodiments, the CMV enhancer comprises a nucleic acid sequence having at least 90% sequence identity to the nucleic acid sequence of any one of SEQ ID NOs: 25-27. In some embodiments, the CMV enhancer comprises a nucleic acid sequence having at least 95% sequence identity to the nucleic acid sequence of any one of SEQ ID NOs: 25-27. In some embodiments, the CMV enhancer comprises a nucleic acid sequence having at least 96% sequence identity to the nucleic acid sequence of any one of SEQ ID NOs: 25-27. In some embodiments, the CMV enhancer comprises a nucleic acid sequence having at least 97% sequence identity to the nucleic acid sequence of any one of SEQ ID NOs: 25-27. In some embodiments, the CMV enhancer comprises a nucleic acid sequence having at least 98% sequence identity to the nucleic acid sequence of any one of SEQ ID NOs: 25-27. In some embodiments, the CMV enhancer comprises a nucleic acid sequence having at least 99% sequence identity to the nucleic acid sequence of any one of SEQ ID NOs: 25-27. In some embodiments, the CMV enhancer comprises a nucleic acid sequence having the nucleic acid sequence of any one of SEQ ID NOs: 25-27. In some embodiments, the CMV enhancer comprises contiguous nucleotides of about 20, about 25, about 40, about 50, about 75, about 100, about 125, about 150, about 175, about 180, about 200, about 220, about 240, about 260, about 280, about 300, about 320, about 340, about 360, about 380, about 400, or about 440 nucleotides of any one of SEQ ID NOs: 25-27. In some embodiments, the CMV enhancer comprises a nucleic acid sequence having at least 70% (e.g., 75%, 80%, 90%, 95%, 96%, 97%, 98%, or 99%) sequence identity to contiguous nucleotides of about 20, about 25, about 40, about 50, about 75, about 100, about 125, about 150, about 175, about 180, about 200, about 220, about 240, about 260, about 280, about 300, about 320, about 340, about 360, about 380, about 400, or about 440 nucleotides of any one of SEQ ID NOs: 25-27.

In some embodiments, the CAG promoter comprises a chicken beta-actin (CBA) gene promoter element. In some embodiments, the CBA gene promoter element comprises a CBA gene promoter sequence, a CBA gene first exon, and a CBA gene first intron. In some embodiments, the CBA promoter comprises the nucleic acid sequence of SEQ ID NO: 28. In some embodiments, the CBA promoter comprises a nucleic acid sequence having at least 70% (e.g., 75%, 80%, 90%, 95%, 96%, 97%, 98%, or 99%) sequence identity to the nucleic acid sequence of SEQ ID NO: 28. In some embodiments, the CBA promoter comprises a nucleic acid sequence having at least 75% sequence identity to the nucleic acid sequence of SEQ ID NO: 28. In some embodiments, the CBA promoter comprises a nucleic acid sequence having at least 80% sequence identity to the nucleic acid sequence of SEQ ID NO: 28. In some embodiments, the CBA promoter comprises a nucleic acid sequence having at least 90% sequence identity to the nucleic acid sequence of SEQ ID NO: 28. In some embodiments, the CBA promoter comprises a nucleic acid sequence having at least 95% sequence identity to the nucleic acid sequence of SEQ ID NO: 28. In some embodiments, the CBA promoter comprises a nucleic acid sequence having at least 96% sequence identity to the nucleic acid sequence of SEQ ID NO: 28. In some embodiments, the CBA promoter comprises a nucleic acid sequence having at least 97% sequence identity to the nucleic acid sequence of SEQ ID NO: 28. In some embodiments, the CBA promoter comprises a nucleic acid sequence having at least 98% sequence identity to the nucleic acid sequence of SEQ ID NO: 28. In some embodiments, the CBA promoter comprises a nucleic acid sequence having at least 99% sequence identity to the nucleic acid sequence of SEQ ID NO: 28. In some embodiments, the CBA promoter comprises a nucleic acid sequence having the nucleic acid sequence of SEQ ID NO: 28. In some embodiments, the CBA promoter comprises contiguous nucleotides of about 20, about 25, about 40, about 50, about 75, about 100, about 125, about 150, about 175, about 180, about 200, about 220, about 240, about 260, about 280, about 300, about 320, about 340, about 360, about 380, about 400, or about 440 nucleotides of SEQ ID NO: 28. In some embodiments, the CBA promoter comprises a nucleic acid sequence having at least 70% (e.g., 75%, 80%, 90%, 95%, 96%, 97%, 98%, or 99%) sequence identity to contiguous nucleotides of about 20, about 25, about 40, about 50, about 75, about 100, about 125, about 150, about 175, about 180, about 200, about 220, about 240, about 260, about 280, about 300, about 320, about 340, about 360, about 380, about 400, or about 440 nucleotides of SEQ ID NO: 28.

In some embodiments, the CAG promoter comprises a spacer sequence immediately 3′ to the CBA promoter element. In some embodiments, the spacer sequence is at least 250 nucleotides in length. In some embodiments the spacer sequence is 250 to 350 nucleotides in length. In some embodiments, the spacer sequence comprises the nucleic acid sequence of SEQ ID NO: 29. In some embodiments, the spacer sequence comprises a nucleic acid sequence having at least 70% (e.g., 75%, 80%, 90%, 95%, 96%, 97%, 98%, or 99%) sequence identity to the nucleic acid sequence of SEQ ID NO: 29. In some embodiments, the spacer sequence comprises a nucleic acid sequence having at least 75% sequence identity to the nucleic acid sequence of SEQ ID NO: 29. In some embodiments, the spacer sequence comprises a nucleic acid sequence having at least 80% sequence identity to the nucleic acid sequence of SEQ ID NO: 29. In some embodiments, the spacer sequence comprises a nucleic acid sequence having at least 90% sequence identity to the nucleic acid sequence of SEQ ID NO: 29. In some embodiments, the spacer sequence comprises a nucleic acid sequence having at least 95% sequence identity to the nucleic acid sequence of SEQ ID NO: 29. In some embodiments, the spacer sequence comprises a nucleic acid sequence having at least 96% sequence identity to the nucleic acid sequence of SEQ ID NO: 29. In some embodiments, the spacer sequence comprises a nucleic acid sequence having at least 97% sequence identity to the nucleic acid sequence of SEQ ID NO: 29. In some embodiments, the spacer sequence comprises a nucleic acid sequence having at least 98% sequence identity to the nucleic acid sequence of SEQ ID NO: 29. In some embodiments, the spacer sequence comprises a nucleic acid sequence having at least 99% sequence identity to the nucleic acid sequence of SEQ ID NO: 29. In some embodiments, the spacer sequence comprises a nucleic acid sequence having the nucleic acid sequence of SEQ ID NO: 29.

In some embodiments, the CAG promoter comprises a rabbit beta-globin splice acceptor. In some embodiments, the rabbit beta-globin splice acceptor comprises the nucleic acid sequence of SEQ ID NO: 30. In some embodiments, the rabbit beta-globin splice acceptor comprises a nucleic acid sequence having at least 70% (e.g., 75%, 80%, 90%, 95%, 96%, 97%, 98%, or 99%) sequence identity to the nucleic acid sequence of SEQ ID NO: 30. In some embodiments, the rabbit beta-globin splice acceptor comprises a nucleic acid sequence having at least 75% sequence identity to the nucleic acid sequence of SEQ ID NO: 30. In some embodiments, the rabbit beta-globin splice acceptor comprises a nucleic acid sequence having at least 80% sequence identity to the nucleic acid sequence of SEQ ID NO: 30. In some embodiments, the rabbit beta-globin splice acceptor comprises a nucleic acid sequence having at least 90% sequence identity to the nucleic acid sequence of SEQ ID NO: 30. In some embodiments, the rabbit beta-globin splice acceptor comprises a nucleic acid sequence having at least 95% sequence identity to the nucleic acid sequence of SEQ ID NO: 30. In some embodiments, the rabbit beta-globin splice acceptor comprises a nucleic acid sequence having at least 96% sequence identity to the nucleic acid sequence of SEQ ID NO: 30. In some embodiments, the rabbit beta-globin splice acceptor comprises a nucleic acid sequence having at least 97% sequence identity to the nucleic acid sequence of SEQ ID NO: 30. In some embodiments, the rabbit beta-globin splice acceptor comprises a nucleic acid sequence having at least 98% sequence identity to the nucleic acid sequence of SEQ ID NO: 30. In some embodiments, the rabbit beta-globin splice acceptor comprises a nucleic acid sequence having at least 99% sequence identity to the nucleic acid sequence of SEQ ID NO: 30. In some embodiments, the rabbit beta-globin splice acceptor comprises a nucleic acid sequence having the nucleic acid sequence of SEQ ID NO: 30.

In some embodiments, the interferon is encoded by the nucleic acid sequence of any one of SEQ ID NOs: 2-4, 6-8, 10-12, and 46. In some embodiments, the interferon is encoded by a nucleic acid sequence having at least 70% (e.g., 75%, 80%, 90%, 95%, 96%, 97%, 98%, or 99%) sequence identity to the nucleic acid sequence of any one of SEQ ID NOs: 2-4, 6-8, 10-12, and 46. In some embodiments, the interferon is encoded by a nucleic acid sequence having at least 75% sequence identity to the nucleic acid sequence of any one of SEQ ID NOs: 2-4, 6-8, 10-12, and 46. In some embodiments, the interferon is encoded by a nucleic acid sequence having at least 80% sequence identity to the nucleic acid sequence of any one of SEQ ID NOs: 2-4, 6-8, 10-12, and 46. In some embodiments, the interferon is encoded by a nucleic acid sequence having at least 90% sequence identity to the nucleic acid sequence of any one of SEQ ID NOs: 2-4, 6-8, 10-12, and 46. In some embodiments, the interferon is encoded by a nucleic acid sequence having at least 95% sequence identity to the nucleic acid sequence of any one of SEQ ID NOs: 2-4, 6-8, 10-12, and 46. In some embodiments, the interferon is encoded by a nucleic acid sequence having at least 96% sequence identity to the nucleic acid sequence of any one of SEQ ID NOs: 2-4, 6-8, 10-12, and 46. In some embodiments, the interferon is encoded by a nucleic acid sequence having at least 97% sequence identity to the nucleic acid sequence of any one of SEQ ID NOs: 2-4, 6-8, 10-12, and 46. In some embodiments, the interferon is encoded by a nucleic acid sequence having at least 98% sequence identity to the nucleic acid sequence of any one of SEQ ID NOs: 2-4, 6-8, 10-12, and 46. In some embodiments, the interferon is encoded by a nucleic acid sequence having at least 99% sequence identity to the nucleic acid sequence of any one of SEQ ID NOs: 2-4, 6-8, 10-12, and 46. In some embodiments, the interferon is encoded by a nucleic acid sequence having the nucleic acid sequence of any one of SEQ ID NOs: 2-4, 6-8, 10-12, and 46.

In some embodiments, the interferon is encoded by the nucleic acid sequence of SEQ ID NO: 4. In some embodiments, the interferon is encoded by a nucleic acid sequence having at least 70% (e.g., 75%, 80%, 90%, 95%, 96%, 97%, 98%, or 99%) sequence identity to the nucleic acid sequence of SEQ ID NO: 4. In some embodiments, the interferon is encoded by a nucleic acid sequence having at least 75% sequence identity to the nucleic acid sequence of SEQ ID NO: 4. In some embodiments, the interferon is encoded by a nucleic acid sequence having at least 80% sequence identity to the nucleic acid sequence of SEQ ID NO: 4. In some embodiments, the interferon is encoded by a nucleic acid sequence having at least 90% sequence identity to the nucleic acid sequence of SEQ ID NO: 4. In some embodiments, the interferon is encoded by a nucleic acid sequence having at least 95% sequence identity to the nucleic acid sequence of SEQ ID NO: 4. In some embodiments, the interferon is encoded by a nucleic acid sequence having at least 96% sequence identity to the nucleic acid sequence of SEQ ID NO: 4. In some embodiments, the interferon is encoded by a nucleic acid sequence having at least 97% sequence identity to the nucleic acid sequence of SEQ ID NO: 4. In some embodiments, the interferon is encoded by a nucleic acid sequence having at least 98% sequence identity to the nucleic acid sequence of SEQ ID NO: 4. In some embodiments, the interferon is encoded by a nucleic acid sequence having at least 99% sequence identity to the nucleic acid sequence of SEQ ID NO: 4. In some embodiments, the interferon is encoded by a nucleic acid sequence having the nucleic acid sequence of SEQ ID NO: 4.

In some embodiments, the minigene comprises the nucleic acid sequence of SEQ ID NO: 32 or SEQ ID NO: 33. In some embodiments, the minigene comprises a nucleic acid sequence having at least 70% (e.g., 75%, 80%, 90%, 95%, 96%, 97%, 98%, or 99%) sequence identity to the nucleic acid sequence of SEQ ID NO: 32 or SEQ ID NO: 33. In some embodiments, the minigene comprises a nucleic acid sequence having at least 75% sequence identity to the nucleic acid sequence of SEQ ID NO: 32 or SEQ ID NO: 33. In some embodiments, the minigene comprises a nucleic acid sequence having at least 80% sequence identity to the nucleic acid sequence of SEQ ID NO: 32 or SEQ ID NO: 33. In some embodiments, the minigene comprises a nucleic acid sequence having at least 90% sequence identity to the nucleic acid sequence of SEQ ID NO: 32 or SEQ ID NO: 33. In some embodiments, the minigene comprises a nucleic acid sequence having at least 95% sequence identity to the nucleic acid sequence of SEQ ID NO: 32 or SEQ ID NO: 33. In some embodiments, the minigene comprises a nucleic acid sequence having at least 96% sequence identity to the nucleic acid sequence of SEQ ID NO: 32 or SEQ ID NO: 33. In some embodiments, the minigene comprises a nucleic acid sequence having at least 97% sequence identity to the nucleic acid sequence of SEQ ID NO: 32 or SEQ ID NO: 33. In some embodiments, the minigene comprises a nucleic acid sequence having at least 98% sequence identity to the nucleic acid sequence of SEQ ID NO: 32 or SEQ ID NO: 33. In some embodiments, the minigene comprises a nucleic acid sequence having at least 99% sequence identity to the nucleic acid sequence of SEQ ID NO: 32 or SEQ ID NO: 33.

In some embodiments, the minigene is regulated by a small molecule splicing modifier. In some embodiments, the small molecule splicing modifier is sudemycin (FR901464, pladienolide B), LMI070, RG7916, or RG7800 and derivatives thereof (e.g., a salt derivative thereof e.g., RG7800-HCl).

In some embodiments, the small molecule splicing modifier is LMI070, also known as branaplam, (CAS No.: 1562338-42-4) having the following structure:

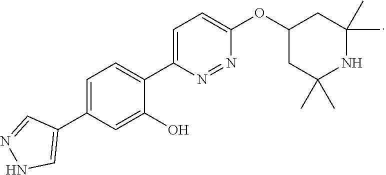 [SMILES: CC1(C)CC(Oc2ccc(-c3ccc(-c4cn[nH]c4)cc3O)nn2)CC(C)(C)N1]

In some embodiments, the viral vector further comprises an antibiotic resistance gene sequence. In some embodiments, the antibiotic resistance gene encodes kanamycin, spectinomycin, streptomycin, ampicillin, carbenicillin, bleomycin, erythromycin, polymyxin B, tetracycline, chloramphenicol, neomycin, zeocin, or a derivative thereof. In some embodiments, the antibiotic resistance gene encodes kanamycin.

In some embodiments, the kanamycin resistance gene comprises a nucleic acid sequence having at least 70% (e.g., 75%, 80%, 90%, 95%, 96%, 97%, 98%, or 99%) sequence identity to the nucleic acid sequence of any one of SEQ ID NOs: 20 or 21. In some embodiments, the kanamycin resistance gene comprises a nucleic acid sequence having at least 75% sequence identity to the nucleic acid sequence of any one of SEQ ID NOs: 20 or 21. In some embodiments, the kanamycin resistance gene comprises a nucleic acid sequence having at least 80% sequence identity to the nucleic acid sequence of any one of SEQ ID NOs: 20 or 21. In some embodiments, the kanamycin resistance gene comprises a nucleic acid sequence having at least 90% sequence identity to the nucleic acid sequence of any one of SEQ ID NOs: 20 or 21. In some embodiments, the kanamycin resistance gene comprises a nucleic acid sequence having at least 95% sequence identity to the nucleic acid sequence of any one of SEQ ID NOs: 20 or 21. In some embodiments, the kanamycin resistance gene comprises a nucleic acid sequence having at least 96% sequence identity to the nucleic acid sequence of any one of SEQ ID NOs: 20 or 21. In some embodiments, the kanamycin resistance gene comprises a nucleic acid sequence having at least 97% sequence identity to the nucleic acid sequence of any one of SEQ ID NOs: 20 or 21. In some embodiments, the kanamycin resistance gene comprises a nucleic acid sequence having at least 98% sequence identity to the nucleic acid sequence of any one of SEQ ID NOs: 20 or 21. In some embodiments, the kanamycin resistance gene comprises a nucleic acid sequence having at least 99% sequence identity to the nucleic acid sequence of any one of SEQ ID NOs: 20 or 21. In some embodiments, the kanamycin resistance gene comprises a nucleic acid sequence having the nucleic acid sequence of any one of SEQ ID NOs: 20 or 21.

In some embodiments, the ampicillin resistance gene comprises a nucleic acid sequence having at least 70% (e.g., 75%, 80%, 90%, 95%, 96%, 97%, 98%, or 99%) sequence identity to the nucleic acid sequence of SEQ ID NO: 34. In some embodiments, the ampicillin resistance gene comprises a nucleic acid sequence having at least 75% sequence identity to the nucleic acid sequence of SEQ ID NO: 34. In some embodiments, the ampicillin resistance gene comprises a nucleic acid sequence having at least 80% sequence identity to the nucleic acid sequence of SEQ ID NO: 34. In some embodiments, the ampicillin resistance gene comprises a nucleic acid sequence having at least 90% sequence identity to the nucleic acid sequence of SEQ ID NO: 34. In some embodiments, the ampicillin resistance gene comprises a nucleic acid sequence having at least 95% sequence identity to the nucleic acid sequence of SEQ ID NO: 34. In some embodiments, the ampicillin resistance gene comprises a nucleic acid sequence having at least 96% sequence identity to the nucleic acid sequence of SEQ ID NO: 34. In some embodiments, the ampicillin resistance gene comprises a nucleic acid sequence having at least 97% sequence identity to the nucleic acid sequence of SEQ ID NO: 34. In some embodiments, the ampicillin resistance gene comprises a nucleic acid sequence having at least 98% sequence identity to the nucleic acid sequence of SEQ ID NO: 34. In some embodiments, the ampicillin resistance gene comprises a nucleic acid sequence having at least 99% sequence identity to the nucleic acid sequence of SEQ ID NO: 34. In some embodiments, the ampicillin resistance gene comprises a nucleic acid sequence having the nucleic acid sequence of SEQ ID NO: 34.

In some embodiments, the rAAV comprises a nucleic acid sequence having at least 70% (e.g., 75%, 80%, 90%, 95%, 96%, 97%, 98%, or 99%) sequence identity to the nucleic acid sequence of SEQ ID NO: 31. In some embodiments, the rAAV comprises a nucleic acid sequence having at least 75% sequence identity to the nucleic acid sequence of SEQ ID NO: 31. In some embodiments, the rAAV comprises a nucleic acid sequence having at least 80% sequence identity to the nucleic acid sequence of SEQ ID NO: 31. In some embodiments, the rAAV comprises a nucleic acid sequence having at least 90% sequence identity to the nucleic acid sequence of SEQ ID NO: 31. In some embodiments, the rAAV comprises a nucleic acid sequence having at least 95% sequence identity to the nucleic acid sequence of SEQ ID NO: 31. In some embodiments, the rAAV comprises a nucleic acid sequence having at least 96% sequence identity to the nucleic acid sequence of SEQ ID NO: 31. In some embodiments, the rAAV comprises a nucleic acid sequence having at least 97% sequence identity to the nucleic acid sequence of SEQ ID NO: 31. In some embodiments, the rAAV comprises a nucleic acid sequence having at least 98% sequence identity to the nucleic acid sequence of SEQ ID NO: 31. In some embodiments, the rAAV comprises a nucleic acid sequence having at least 99% sequence identity to the nucleic acid sequence of SEQ ID NO: 31. In some embodiments, the rAAV comprises a nucleic acid sequence having the nucleic acid sequence of SEQ ID NO: 31.

In some embodiments, the nucleic acids comprise those sequences of AAV that are required in cis for replication and packaging (e.g., functional ITRs) of the DNA into a virion. In some embodiments, the rAAV vectors contain marker or reporter genes.

Useful rAAV vectors comprise those having one or more of the naturally occurring AAV genes deleted in whole or in part, but retain functional flanking ITR sequences. In some embodiments, the AAV ITRs are of any serotype suitable for a particular application. In some embodiments, the nucleic acid does not comprise ITRs. In some embodiments, the AAV ITRs are of any suitable AAV serotype, comprising any now known or later discovered serotypes or any engineered, evolved, selected, or chimeric capsid serotype.

The capsid proteins of AAV compose the exterior, non-nucleic acid portion of the virion and are encoded by the AAV Cap gene. In some embodiments, the Cap gene encodes VP1, VP2, VP3, MAAP, AAP, or combinations thereof.

In some embodiments, provided herein are Rep, Cap, or other polynucleotides required for producing the rAAV of the disclosure. In some embodiments, the Rep, Cap, or other polynucleotides are delivered to the packaging host cell using any appropriate genetic element (e.g., vector). In some embodiments, a single nucleic acid encoding all three capsid proteins (e.g., VP1, VP2 and VP3) is delivered into the packaging host cell in a single vector. In some embodiments, nucleic acids encoding the capsid proteins are delivered into the packaging host cell by two vectors; a first vector comprising a first nucleic acid encoding two capsid proteins (e.g., VP1 and VP2) and a second vector comprising a second nucleic acid encoding a single capsid protein (e.g., VP3). In some embodiments, three vectors, each comprising a nucleic acid encoding a single, different capsid protein, are delivered to the packaging host cell.

In some embodiments, a single nucleic acid encoding multiple replication proteins (e.g., Rep78, Rep68, Rep52, and Rep40) is delivered into the packaging host cell in a single vector. In some embodiments, nucleic acids encoding the replication proteins are delivered into the packaging host cell by two vectors; a first vector comprising a first nucleic acid encoding one to three replication proteins and a second vector comprising a second nucleic acid encoding one to three replication proteins. In some embodiments, four vectors, each comprising a nucleic acid encoding a single, different replication protein, are delivered to the packaging host cell.

In some embodiments, a single nucleic acid encoding multiple adenovirus helper proteins (e.g., E1A, E1B, E4, E2A, and VA RNA) is delivered into the packaging host cell in a single vector. In some embodiments, nucleic acids encoding the adenovirus helper proteins are delivered into the packaging host cell by two vectors. In some embodiments, more than two vectors, each comprising a nucleic acid encoding a different capsid protein, are delivered to the packaging host cell. The selected genetic elements are delivered by any suitable method, comprising those described herein. The methods used to construct any embodiment of this disclosure comprise genetic engineering, recombinant engineering, and synthetic techniques.

In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16.

In some embodiments, a rAAV is AAV1 or a derivative thereof. In some embodiments, a rAAV is AAV2 or a derivative thereof. In some embodiments, a rAAV is AAV3 or a derivative thereof. In some embodiments, a rAAV is AAV4 or a derivative thereof. In some embodiments, a rAAV is AAV5 or a derivative thereof. In some embodiments, a rAAV is AAV6 or a derivative thereof. In some embodiments, a rAAV is AAV7 or a derivative thereof. In some embodiments, a rAAV is AAV8 or a derivative thereof. In some embodiments, a rAAV is AAV9 or a derivative thereof. In some embodiments, a rAAV is AAV10 or a derivative thereof. In some embodiments, a rAAV is AAV11 or a derivative thereof. In some embodiments, a rAAV is AAV12 or a derivative thereof. In some embodiments, a rAAV is AAV13 or a derivative thereof. In some embodiments, a rAAV is AAV14 or a derivative thereof. In some embodiments, a rAAV is AAV15 or a derivative thereof. In some embodiments, a rAAV is AAV16 or a derivative thereof. In some embodiments, a rAAV is AAV-rh8 or a derivative thereof. In some embodiments, a rAAV is AAV-rh10 or a derivative thereof. In some embodiments, a rAAV is AAV-rh20 or a derivative thereof. In some embodiments, a rAAV is AAV-rh39 or a derivative thereof. In some embodiments, a rAAV is AAV-rh74 or a derivative thereof. In some embodiments, a rAAV is AAV-rhM4-1 or a derivative thereof. In some embodiments, a rAAV is AAV-hu37 or a derivative thereof. In some embodiments, a rAAV is AAV-Anc80 or a derivative thereof. In some embodiments, a rAAV is AAV-Anc80L65 or a derivative thereof. In some embodiments, a rAAV is AAV-7m8 or a derivative thereof. In some embodiments, a rAAV is AAV-PHP-B or a derivative thereof. In some embodiments, a rAAV is AAV-PHP-EB or a derivative thereof. In some embodiments, a rAAV is AAV-2.5 or a derivative thereof. In some embodiments, a rAAV is AAV-2tYF or a derivative thereof. In some embodiments, a rAAV is AAV-3B or a derivative thereof. In some embodiments, a rAAV is AAV-LK03 or a derivative thereof. In some embodiments, a rAAV is AAV-HSC1 or a derivative thereof. In some embodiments, a rAAV is AAV-HSC2 or a derivative thereof. In some embodiments, a rAAV is AAV-HSC3 or a derivative thereof. In some embodiments, a rAAV is AAV-HSC4 or a derivative thereof. In some embodiments, a rAAV is AAV-HSC5 or a derivative thereof. In some embodiments, a rAAV is AAV-HSC6 or a derivative thereof. In some embodiments, a rAAV is AAV-HSC7 or a derivative thereof. In some embodiments, a rAAV is AAV-HSC8 or a derivative thereof. In some embodiments, a rAAV is AAV-HSC9 or a derivative thereof. In some embodiments, a rAAV is AAV-HSC10 or a derivative thereof. In some embodiments, a rAAV is AAV-HSC11 or a derivative thereof. In some embodiments, a rAAV is AAV-HSC12 or a derivative thereof. In some embodiments, a rAAV is AAV-HSC13 or a derivative thereof. In some embodiments, a rAAV is AAV-HSC14 or a derivative thereof. In some embodiments, a rAAV is AAV-HSC15 or a derivative thereof. In some embodiments, a rAAV is AAV-TT or a derivative thereof. In some embodiments, a rAAV is AAV-DJ/8 or a derivative thereof. In some embodiments, a rAAV is AAV-Myo or a derivative thereof. In some embodiments, a rAAV is AAV-NP40 or a derivative thereof. In some embodiments, a rAAV is AAV-NP59 or a derivative thereof. In some embodiments, a rAAV is AAV-NP22 or a derivative thereof. In some embodiments, a rAAV is AAV-NP66 or a derivative thereof. In some embodiments, a rAAV is AAV-HSC16 or a derivative thereof.

In some embodiments, the rAAV vector is a pseudotyped rAAV vector. Pseudotyped vectors comprise AAV vectors of a given serotype pseudotyped with a Cap gene derived from a serotype other than the given serotype (e.g., AAV1, AAV3, AAV4, AAV5, AAV6, AAV7, AAV8, or AAV9, among others). For example, a representative pseudotyped vector is an AAV2 vector pseudotyped with a Cap gene derived from AAV serotype 8 or AAV serotype 9.

In some embodiments, the AAV have mutations within the virion capsid that are used to transduce particular cell types more effectively than non-mutated capsid virions. In some embodiments, suitable AAV mutants have ligand insertion mutations for the facilitation of targeting AAV to specific cell types. In some embodiments, the construction and characterization of AAV capsid mutants comprising insertion mutants, alanine screening mutants, and epitope tag mutants are used.

In some embodiments, artificial AAV capsids are used. Such an artificial capsid can be generated by any suitable technique using a selected AAV sequence (e.g., a fragment of a VPl capsid protein) in combination with heterologous sequences which are obtained from a different selected AAV serotype, non-contiguous portions of the same AAV serotype, from a non-AAV viral source, or from a non-viral source. An artificial AAV serotype is, without limitation, a pseudotyped AAV, a chimeric AAV capsid, a recombinant AAV capsid, or a “humanized” AAV capsid.

Other rAAV virions that are used in compositions and methods of the disclosure include, but are not limited to, those capsid hybrids that are generated by molecular breeding of viruses as well as by exon shuffling or generated through use of artificial intelligence or machine learning.

In some embodiments, the capsid is modified to improve therapy. In some embodiments, the capsid is modified for minimized immunogenicity, better stability and particle durability, efficient degradation, and/or accurate delivery of the heterologous coding sequence or a functional fragment or variant thereof to the nucleus. In some embodiments, the modification or mutation is an amino acid deletion, insertion, substitution, or any combination thereof in a capsid polypeptide. In some embodiments, the capsid polypeptide comprises 1, 2, 3, 4, 5, up to 10, or more amino acid substitutions and/or deletions and/or insertions. In some embodiments, one or more amino acid substitutions are introduced into one or more of VP1, VP2 and VP3. In some embodiments, a modified capsid polypeptide comprises 1, 2, 3, 4, 5, 6, 7, 8, 9, 10, 11, 12, 13, 14, or 15 conservative or non-conservative substitutions relative to the wild-type polypeptide.

In another embodiment, the modified capsid polypeptide of the disclosure comprises modified sequences, wherein such modifications can comprise both conservative and non-conservative substitutions, deletions, and/or additions, and typically comprise peptides that share at least 60%, at least 65%, at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% sequence identity to the corresponding wild-type capsid protein.

Methods for Generating rAAV Vectors

Described herein, are methods for generating rAAV vectors, comprising providing cells with the nucleic acids described herein, wherein the cells are transfected with the nucleic acids. In some embodiments, the cells are infected with the nucleic acids. In some embodiments, the cells are transduced with the nucleic acids. In some embodiments, the cells are in suspension. In some embodiments, the cells are adherent. In some embodiments, the cells are engineered stable cell lines.

In some embodiments, the cell is a eukaryotic cell (e.g., a plant cell, an animal cell, a protist cell, or a fungi cell), a mammalian cell (a Chinese hamster ovary (CHO) cell, baby hamster kidney (BHK), human embryo kidney (HEK-293), mouse myeloma (NS0), Vero, or human retinal cells), an immortalized cell (e.g., a HeLa cell, a COS cell, a HEK-293T cell, a MDCK cell, a 3T3 cell, a PC12 cell, a Huh7 cell, a HepG2 cell, a K562 cell, a N2a cell, or a SY5Y cell), an insect cell (e.g., a Spodoptera frugiperda cell, a Trichoplusiani cell, a Drosophila melanogaster cell, a S2 cell, or a Heliothis virescens cell), a yeast cell (e.g., a Saccharomyces cerevisiae cell, a Cryptococcus cell, or a Candida cell), a plant cell (e.g., a parenchyma cell, a collenchyma cell, or a sclerenchyma cell), a fungal cell (e.g., a Saccharomyces cerevisiae cell, a Cryptococcus cell, or a Candida cell), or a prokaryotic cell (e.g., a E. coli cell, a streptococcus bacterium cell, a streptomyces soil bacteria cell, or an archaea cell). In some embodiments, the cell is a eukaryotic cell. In some embodiments, the cell is a mammalian cell. In some embodiments, the cell is an immortalized cell. In some embodiments, the cell is an insect cell. In some embodiments, the cell is a yeast cell. In some embodiments, the cell is a plant cell. In some embodiments, the cell is a fungal cell. In some embodiments, the cell is a prokaryotic cell.

In some embodiments, the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, a primary cell, or derivative thereof. In some embodiments, the cell is a A549 cell or a derivative thereof. In some embodiments, the cell is a HEK-293 cell or a derivative thereof. In some embodiments, the cell is a HEK-293T cell or a derivative thereof. In some embodiments, the cell is a BHK cell or a derivative thereof. In some embodiments, the cell is a CHO cell or a derivative thereof. In some embodiments, the cell is a HeLa cell or a derivative thereof. In some embodiments, the cell is a MRC5 cell or a derivative thereof. In some embodiments, the cell is a Sf9 cell or a derivative thereof. In some embodiments, the cell is a Cos-1 cell or a derivative thereof. In some embodiments, the cell is a Cos-7 cell or a derivative thereof. In some embodiments, the cell is a Vero cell or a derivative thereof. In some embodiments, the cell is a BSC 1 cell or a derivative thereof. In some embodiments, the cell is a BSC 40 cell or a derivative thereof. In some embodiments, the cell is a BMT 10 cell or a derivative thereof. In some embodiments, the cell is a WI38 cell or a derivative thereof. In some embodiments, the cell is a HeLa cell or a derivative thereof. In some embodiments, the cell is a Saos cell or a derivative thereof. In some embodiments, the cell is a C2C12 cell or a derivative thereof. In some embodiments, the cell is a L cell or a derivative thereof. In some embodiments, the cell is a HT1080 cell or a derivative thereof. In some embodiments, the cell is a HepG2 cell or a derivative thereof. In some embodiments, the cell is a Huh7 cell or a derivative thereof. In some embodiments, the cell is a K562 cell or a derivative thereof. In some embodiments, the cell is a primary cell.

In some embodiments, the nucleic acids encoding one or more interferons described herein are expressed transiently. In some embodiments, the nucleic acids encoding one or more interferons described herein are expressed transiently from an inducible system. In some embodiments, the nucleic acids encoding one or more interferons described herein are stably expressed. In some embodiments, the nucleic acids encoding one or more interferons described herein are expressed stably from an inducible system.

In some embodiments, the recombinant viral vectors are produced by a producer cell line method. Briefly, for example, a cell line (e.g., a HEK-293 cell line) is stably transfected with a plasmid containing a Rep gene, a Cap gene, and a promoter-payload sequence. Cell lines are screened to select a lead clone for recombinant vector production, which in some embodiments, are expanded to a production bioreactor and infected with, a helper polynucleotide (e.g., a wild-type adenovirus) as a helper to initiate vector production. In some embodiments, the recombinant viral vector (e.g., rAAV) is subsequently harvested, the adenovirus is inactivated (e.g., by heat) and/or removed, and the viral particles are purified. In some embodiments, recombinant viral vectors are purified and formulated.

In some embodiments, suitable media is used for the production of recombinant vectors. These media comprise, without limitation, media produced by Hyclone Laboratories and JRH comprising Modified Eagle Medium (MEM), Roswell Park Memorial Institute (RPMI) 1640, Eagle's Minimal Essential Medium (EMEM), Dulbecco's Modified Eagle Medium (DMEM), and custom formulations, particularly with respect to custom media formulations for use in production of recombinant vectors.

In some embodiments, suitable production culture media of the present disclosure is supplemented with serum or serum-derived recombinant proteins at a level of 0.5-20 (v/v or w/v). In some embodiments, vectors are produced in serum-free conditions which are also referred to as media with no animal-derived products. In some embodiments, commercial or custom media is designed to support production of vectors, comprising supplementation of, without limitation glucose, vitamins, amino acids, and or growth factors, in order to increase the titer and/or yield of vector in production cultures.

Vector production cultures comprise a variety of conditions (over a wide temperature range, for varying lengths of time, and the like) suitable to the particular host cell being utilized. Vector production cultures comprise attachment-dependent cultures which are cultured in suitable attachment-dependent vessels such as, for example, plates, flasks, cell stacks, roller bottles, hollow fiber filters, microcarriers, and packed-bed or fluidized-bed bioreactors. In some embodiments, vector production cultures comprise suspension-adapted host cells such as HeLa, HEK-293, and SF-9 cells which are cultured in a variety of ways comprising, for example, spinner flasks, stirred tank bioreactors, single use bioreactors such as Cytiva Xcellerex, Sartorius, and Wave, and disposable systems.

In some embodiments, viral particles of the disclosure are harvested from vector production cultures by lysis of the host cells of the production culture or by harvest of the spent media from the production culture, provided the cells are cultured under conditions to cause release of viral particles into the media from intact cells. Suitable methods of lysing cells comprise, for example, multiple freeze/thaw cycles, sonication, microfluidization, and treatment with chemicals, such as detergents and/or proteases.

In a further embodiment, the viral particles are purified. The term “purified” as used herein comprises a preparation of viral particles devoid of at least some of the other components that are present where the viral particles naturally occur or are initially prepared from. Thus, for example, in some embodiments, isolated viral particles are prepared using a purification technique to enrich it from a source mixture, such as a culture lysate or production culture supernatant. In some embodiments, enrichment is measured in a variety of ways, such as, for example, by the proportion of dNase-resistant particles (DRPs) or genome copies (gc) present in a solution, or by infectivity, or measured in relation to a second, potentially interfering substance present in the source mixture, such as contaminants and impurities, comprising product-derived and process-derived impurities and the like.

In some embodiments, the vector production culture harvest is clarified to remove host cell debris. In some embodiments, the production culture harvest is clarified by filtration through a series of depth filters comprising, for example, a grade DOHC Millipore Millistak+HC Pod Filter, a grade A1HC Millipore Millistak+HC Pod Filter, and a 0.2 μm Filter Opticap XL 10 Millipore Express SHC Hydrophilic Membrane filter. Clarification can also be achieved by a variety of other standard techniques, such as, centrifugation or filtration through any cellulose acetate filter of 0.2 μm or greater pore size.

In some embodiments, the vector production culture harvest is further treated with a nuclease (e.g., DNA or RNA nuclease) to digest any high molecular weight DNA present in the production culture. In some embodiments, the nuclease digestion is performed under standard conditions.

In some embodiments, viral particles are isolated or purified using one or more of the following purification steps: equilibrium centrifugation; flow-through anion exchange filtration; tangential flow filtration (TFF) for concentrating the viral particles; vector capture by apatite chromatography; heat inactivation of helper virus; vector capture by hydrophobic interaction chromatography; buffer exchange by size exclusion chromatography (SEC); nanofiltration; vector capture by anion exchange chromatography, cation exchange chromatography, or affinity chromatography. In some embodiments, these steps are used alone, in various combinations, or in different orders. In some embodiments, the method comprises all the steps in the order as described below.

Described herein, are methods for generating a recombinant vector, wherein the methods comprise providing cells transfected with a helper polynucleotide. In some embodiments, the cells are transfected with a helper polynucleotide that provides helper functions to the AAV. In some embodiments, the helper polynucleotide provides adenovirus functions, comprising, e.g., E1A, E1B, E2A, E4ORF6, and/or VA RNA. The sequences of adenovirus genes providing these functions, in some embodiments, are obtained from any known adenovirus serotype, such as serotypes 2, 3, 4, 7, 12, and 40, and further comprising any of the presently identified human types. In some embodiments, the methods involve transfecting the cell with vectors expressing one or more genes necessary for AAV replication, AAV gene transcription, and/or AAV packaging.

Described herein, are methods for generating a recombinant vector, wherein the methods comprise providing cells transfected with a helper polynucleotide that is under the control of a promoter. In some embodiments, the cells are stable host cells comprising the required component(s) under the control of an inducible promoter. In some embodiments, the cells are stable host cells comprising the required component(s) under the control of a constitutive promoter. In some embodiments, the cells are stable host cells comprising the selected components under the control of a constitutive promoter and other selected components under the control of one or more inducible promoters. For example, a stable host cell is generated which is derived from HEK-293 cells (which contain El helper functions under the control of a constitutive promoter), and which contains the Rep and/or Cap proteins under the control of inducible promoters.

In some embodiments, the minigene, Rep sequences, Cap sequences, and helper functions required for producing the rAAV of the disclosure are delivered to the packaging host cell in the form of any genetic element which transfers the sequences. In some embodiments, the selected genetic elements are delivered by any suitable method.

Production of recombinant vectors, such as rAAV vectors, in some embodiments, comprises transfection, stable cell line production, and infectious hybrid virus production systems which comprise adenovirus-AAV hybrids, herpesvirus-AAV hybrids, and baculovirus-AAV hybrids.

Methods comprising transfection, stable cell line production, and infectious hybrid virus production systems, in some embodiments, are used for production of rAAV vectors. In some embodiments, the methods described herein comprise adenovirus-AAV hybrids, herpesvirus-AAV hybrids and baculovirus-AAV hybrids. rAAV production cultures for the production of rAAV virus particles all require; 1) suitable host cells, comprising, for example, human-derived cell lines such as HeLa, A549, or HEK-293 cells, or insect-derived cell lines such as SF-9, in the case of baculovirus production systems, or transgenic plant cells; 2) suitable helper virus function, provided by wild-type or mutant adenovirus (such as temperature-sensitive adenovirus), herpes virus, baculovirus, or a plasmid construct providing helper functions; 3) AAV Rep and Cap genes and gene products; 4) a desired genomic payload (such as a heterologous sequence encoding any desired sequence or fragment or variant thereof) flanked by at least one AAV ITR sequence; and 5) suitable media and media components to support rAAV production. Suitable media for the production of rAAV vectors comprise, without limitation, media produced by Hyclone Laboratories and JRH comprising Modified Eagle Medium (MEM), Roswell Park Memorial Institute (RPMI) 1640, Eagle's Minimal Essential Medium (EMEM), Dulbecco's Modified Eagle Medium (DMEM), and custom formulations, such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of recombinant AAV vectors.

In some embodiments, rAAV particles are generated by transfecting producer cells with a plasmid (cis plasmid) containing a rAAV genome comprising a payload flanked by one or more AAV ITRs and a separate construct expressing the AAV Rep and Cap genes in trans. In some embodiments, adenovirus helper factors such as E1A, E1B, E2A, E4ORF6, and VA RNAs are provided by either adenovirus infection or by transfecting a third plasmid providing adenovirus helper polynucleotides into the producer cells. In some embodiments, producer cells are HEK-293 cells. In some embodiments, the helper polynucleotides provided will vary depending on the producer cells used and whether the producer cells already carry some of these helper polynucleotides.

In some embodiments, the rAAV vectors described herein are produced by a triple transfection method, such as the exemplary triple transfection method provided infra. In some embodiments, the plasmid containing a Rep gene and a Cap gene, along with a helper adenoviral plasmid, is transfected (e.g., using squeeze-poration, lipofection, optical transfection, the calcium phosphate method, or polyethylenimine (PEI)) into a cell line (e.g., HEK-293 cells), and virus is collected and optionally purified.

In some embodiments, rAAV vectors are produced by a producer cell line method, such as the exemplary producer cell line method provided infra. Briefly, a cell line (e.g., a HEK-293 cell line) is stably transfected with a plasmid containing a Rep gene, a Cap gene, and a promoter-payload sequence. Cell lines are screened to select a lead clone for rAAV production, which are then expanded to a production bioreactor and infected with an adenovirus (e.g., a wild-type adenovirus) as helper to initiate rAAV production. Virus is subsequently harvested, adenovirus may be inactivated (e.g., by heat) and/or removed, and the rAAV vectors are purified.

In some embodiments, a method is provided for producing any rAAV vector as disclosed herein comprising: (a) culturing a host cell under a condition that rAAV vectors are produced, wherein the host cell comprises: (i) one or more AAV packaging genes, wherein each said AAV packaging gene encodes an AAV replication and/or encapsidation protein; (ii) a rAAV pro-vector comprising nucleic acids encoding a therapeutic polypeptide and/or nucleic acid as described herein flanked by at least one AAV ITR; and (iii) an AAV helper function; and (b) recovering the rAAV vectors produced by the host cell. In some embodiments, said at least one AAV ITR is selected from the group consisting of AAV ITRs derived from AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, AAV-HSC16, a goat AAV, bovine AAV, or mouse AAV, or the like. In some embodiments, the replication protein is an AAV2 replication protein.

### Methods of Treating Cancer

Described herein are methods of treating cancer in a subject in need thereof, comprising: a) administering a nucleic acid comprising a first polynucleotide comprising a minigene 5′ of a second polynucleotide encoding an interferon, wherein the minigene comprises a splicing binding site; and b) administering a small molecule splice modulator for splicing the minigene of step a). In some embodiments, administration includes administering an rAAV vector, an isolated cell, or a pharmaceutical composition comprising the nucleic acids described herein to a subject.

In some embodiments, the subject is a human. In some embodiments, the subject has cancer. In some embodiments, the subject has a glioma, such as a grade III or grade IV glioma (glioblastoma). In some embodiments, the subject has glioblastoma. In some embodiments, the subject has a primary tumor. In some embodiments, the subject has a metastase.

In some embodiments, administration is to the central nervous system. In some embodiments, administration is to a brain. In some embodiments, administration is to a brain ventricle. In some embodiments, administration is intratumoral.

Any suitable route of administration or combination of different routes can be used, including systemic administration (e.g., intravenous, intravascular, and intraarterial), local injection into the central nervous system (CNS; e.g., intratumoral injection; intracranial injection; intracerebral injection; intracerebroventricular; or injection into the cerebrospinal fluid (CSF) via the cerebral ventricular system, cisterna magna, or intrathecal space), or local injection at other bodily sites (e.g., intraocular, intramuscular, subcutaneous, intradermal injection, and transdermal). In some embodiments, the administration is by intratumoral injection, intracranial injection, intracerebral injection, intracerebroventricular, or injection into the CSF via the cerebral ventricular system, cisterna magna, or intrathecal space.

In some embodiments, intracerebroventricular injection occurs in the right lateral ventricle, left lateral ventricle, third ventricle, fourth ventricle, interventricular foramina (also called the foramina of Monro), cerebral aqueduct, central canal, median aperture, right lateral aperture, left lateral aperture, perivascular space, or the subarachnoid space.

Administration can be performed by use of an osmotic pump, by electroporation, or by other means. In some embodiments, administration of the nucleic acids described herein are performed before, after, or simultaneously with surgical tumor removal or biopsy.

In some embodiments, the administration is by convection enhanced delivery (CED). CED uses direct infusion of a drug-containing liquid into tissue so that transport is dominated by convection. In some embodiments, the device is an osmotic pump. In some embodiments, the device is an infusion pump. In some embodiments, CED is performed with a step-design cannula.

In some embodiments, magnetic resonance imaging (MRI)-guided CED is performed to deliver nucleic acids of the present disclosure. In some embodiments, CED further comprises the use of a tracing agent. In some embodiments, the tracing agent is an MRI contrast enhancing agent. In some embodiments, the MRI contrast enhancing agent is gadolinium or related chemical derivatives. In some embodiments, the MRI contrast enhancing agent and the nucleic acids are administered simultaneously. In some embodiments, the MRI contrast enhancing agent is mixed with the nucleic acids directly prior to administration.

### Pharmaceutical Compositions

Described herein, in some embodiments, are pharmaceutical compositions comprising the nucleic acids of the disclosure or rAAV comprising the nucleic acids of the disclosure. A composition or a medicament of the disclosure is in a form suitable for administration to an individual in need thereof.

According to a particular embodiment, the pharmaceutically acceptable excipient compositions of the disclosure are suitably selected from the group consisting of an injectable excipient liquid such as sterile water for injection and an aqueous solution, such as saline.

Acceptable excipients are physiologically acceptable to be administered to a subject and retain the therapeutic properties of the compounds with/in which it is administered. Acceptable excipients and their formulations are generally described in, for example, Remington's Pharmaceutical Sciences, supra. One exemplary excipient is physiological saline. The phrase “pharmaceutically acceptable excipient” as used herein means a pharmaceutically acceptable material, composition or vehicle, such as a liquid or solid filler, diluent, excipient, solvent or encapsulating material, involved in carrying or transporting the active compound from administration to a site, such as an organ, portion of the body, or in an in vitro assay system. Each excipient is acceptable in the sense of being compatible with the other ingredients of the formulation and not injurious to a subject to whom it is administered. Nor should an acceptable excipient alter the specific activity of the subject compounds.

In another embodiment, pharmaceutical compositions disclosed herein further comprise an acceptable additive to improve the stability of the compounds in composition and/or to control the release rate of the compositions. Acceptable additives do not alter the specific activity of the subject compounds. Exemplary acceptable additives include, but are not limited to, a sugar such as mannitol, sorbitol, glucose, xylitol, trehalose, sorbose, sucrose, galactose, dextran, dextrose, fructose, lactose, and mixtures thereof. Acceptable additives are combined with acceptable carriers and/or excipients such as dextrose in some embodiments. Alternatively, exemplary acceptable additives include, but are not limited to, a surfactant such as polysorbate 20 or polysorbate 80 to increase stability of a peptide and decrease gelling of a solution. In some embodiments, the surfactant is added to a composition in an amount of 0.01% to 5% of the solution. Addition of such acceptable additives increases the stability and half-life of the composition in storage.

Suspensions, lyophilized, and crystal forms of nucleic acids or compositions herein are also contemplated herein; methods to make suspensions, lyophilizations, and crystal forms are known to one of skill in the art.

In some embodiments, pharmaceutical compositions disclosed herein are sterile. In some embodiments, pharmaceutical compositions disclosed herein are sterilized by conventional, well known sterilization techniques. For example, sterilization is readily accomplished by filtration through sterile filtration membranes. In some embodiments, the resulting solution is packaged for use or filtered under aseptic conditions and lyophilized, the lyophilized preparation being combined with a sterile solution prior to administration.

Freeze-drying is employed to stabilize polypeptides for long-term storage, such as when a polypeptide is relatively unstable in liquid compositions, in some embodiments.

In some embodiments, some excipients such as, for example, polyols (including mannitol, sorbitol, and glycerol); sugars (including glucose, sucrose, and trehalose); and amino acids (including alanine, glycine, and glutamic acid), act as stabilizers for freeze-dried products. Polyols and sugars are also used to protect polypeptides from freezing- and drying-induced damage and to enhance the stability during storage in the dried state, in some embodiments. Sugars are, in some embodiments, effective in both the freeze-drying process and during storage. Other classes of molecules, including mono- and disaccharides and polymers such as PVP, have also been reported as stabilizers of lyophilized products.

For injection, in some embodiments, pharmaceutical compositions disclosed herein are in a powder suitable for reconstitution with an appropriate solution as described above. Examples of these include, but are not limited to, freeze-dried, rotary-dried, or spray-dried powders, amorphous powders, granules, precipitates, or particulates. For injection, the compositions herein optionally contain stabilizers, pH modifiers, surfactants, bioavailability modifiers, and combinations of these.

In some embodiments, sustained-release preparations are prepared. Suitable examples of sustained-release preparations include semipermeable matrices of solid hydrophobic polymers containing pharmaceutical compositions herein, which matrices are in the form of shaped articles, e.g., films or microcapsule. Examples of sustained-release matrices include polyesters, hydrogels (for example, poly(2-hydroxyethyl-methacrylate), or poly(vinylalcohol)), polylactides (see, e.g., U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

In some embodiments, pharmaceutical compositions disclosed herein are designed to be short-acting, fast-releasing, long-acting, or sustained-releasing as described herein. In some embodiments, pharmaceutical compositions disclosed herein are formulated for controlled release or for slow release.

In some embodiments, the pharmaceutical compositions herein are comprised in a container, pack, or dispenser together with instructions for administration.

### Examples

Below are examples of specific embodiments for carrying out the present disclosure. The examples are offered for illustrative purposes only and are not intended to limit the scope of the present disclosure in any way. Efforts have been made to ensure accuracy with respect to numbers used (e.g., amounts, temperatures, etc.), but some experimental error and deviation should, of course, be allowed for.

### Example 1. Generation of Viral Vectors

This Example describes the generation of nucleic acids including a splice modulator binding of the disclosure.

Adeno-associated virus (AAV) expression vectors including a splice modulator binding site (“X^{on} splicing control element” or “X^{on}”) with various payloads were generated (Table 1). All the vectors containing a minigene including a splice modulator binding site were modified at the DNA level such that the payloads had the first ATG start codon removed, as well as all potential alternative in-frame start codon(s) altered to maintain the amino acid but to not be a start codon. The protein sequence of each payload was unchanged. Plasmids containing the various AAV genome sequences were cloned and used to produce the corresponding AAV vector stocks (exemplified in Table 2). Constructs with a matching payload sequence but without the X^{on} splicing control element were also engineered for each new X^{on}-containing AAV, to serve as a constitutive expression control (not shown). A series of initial test payloads were designed to verify and quantify payload expression with and without splice induction, including: enhanced green fluorescent protein (eGFP), degradable eGFP (d2eGFP), firefly Luciferase (fLuc), NanoLuc Luciferase (nLuc), mCardinal (mCard), and CpG-depleted human interferon beta (hIFNβ-CpGdel).

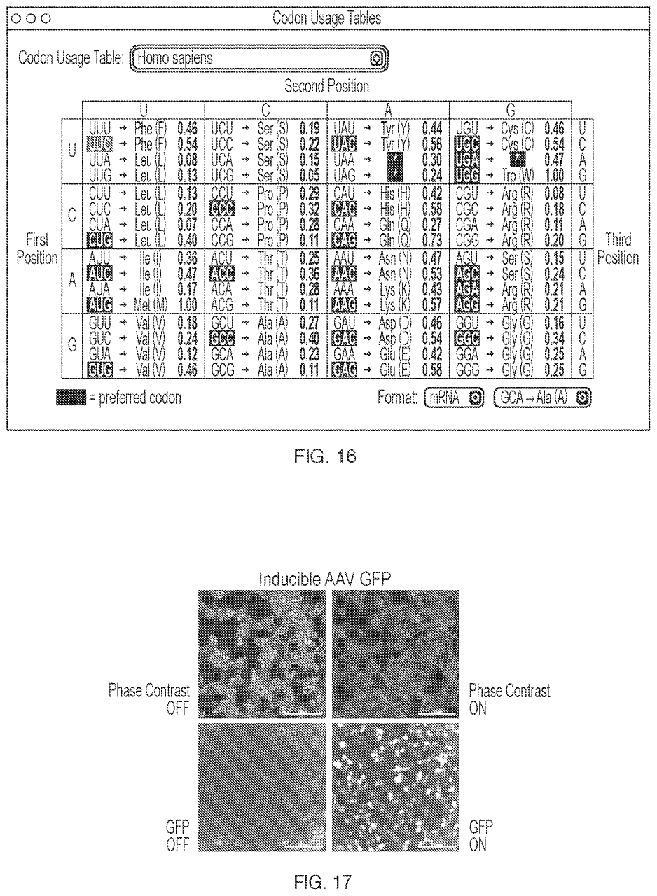

To generate CpG-depleted human interferon beta, every CpG present in the nucleotide sequence of wildtype human interferon beta were identified and each C or G of a CpG duplex in the gene was removed and replaced with the appropriate nucleotide to maintain the amino acid codon sequence, using the human codon usage table depicted in FIG. 16.

Taken together, these successful cloning experiments and viral productions demonstrated that high viral genome titers were obtained (>1E13 vector genomes (vg)/mL) for each AAV construct containing the X^{on} splicing control element, as well as the suitability of the newly designed genomes for AAV packaging and transduction experiments, as well as robust, and clinically relevant levels of viral production.

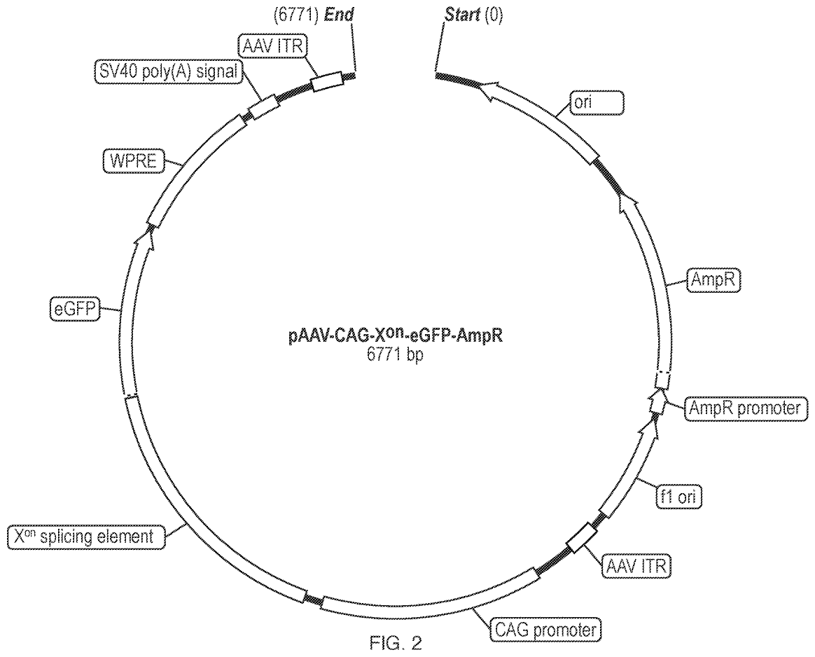

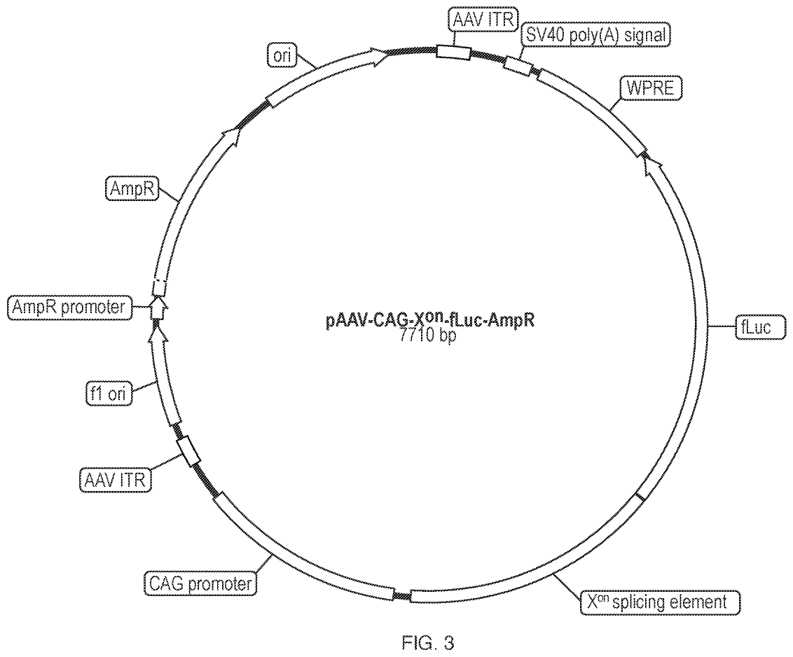

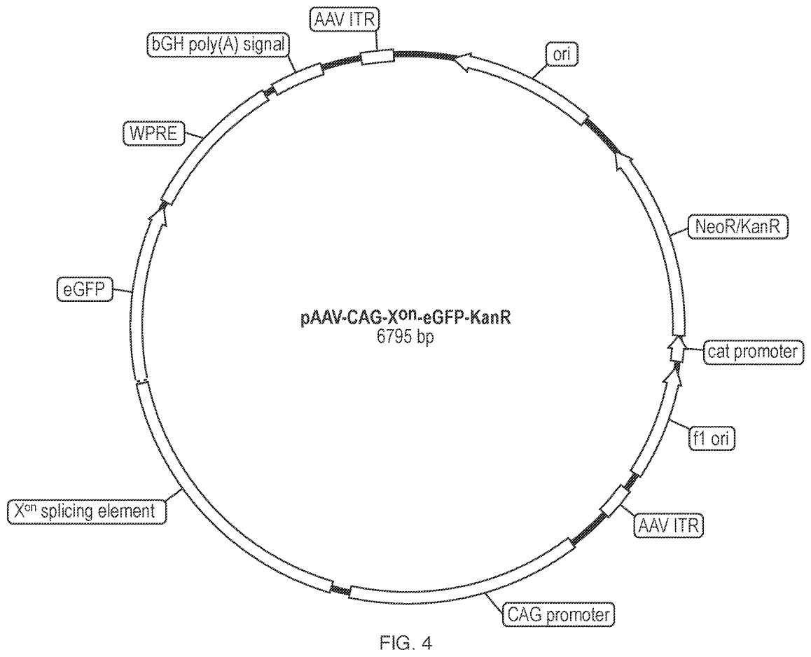

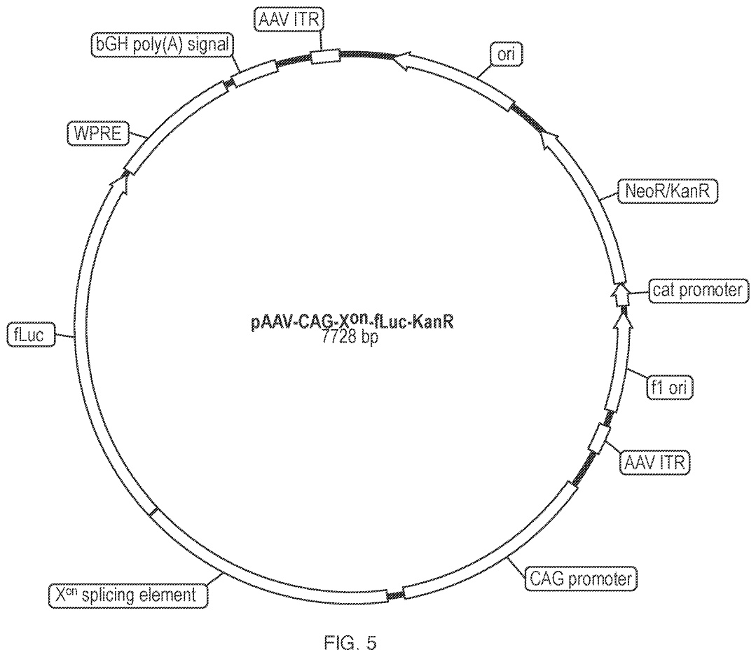

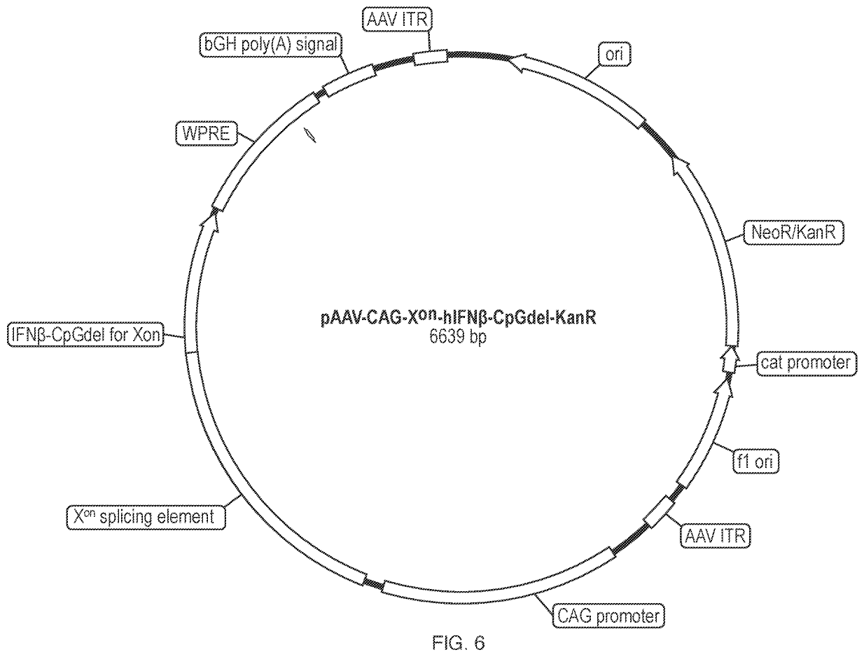

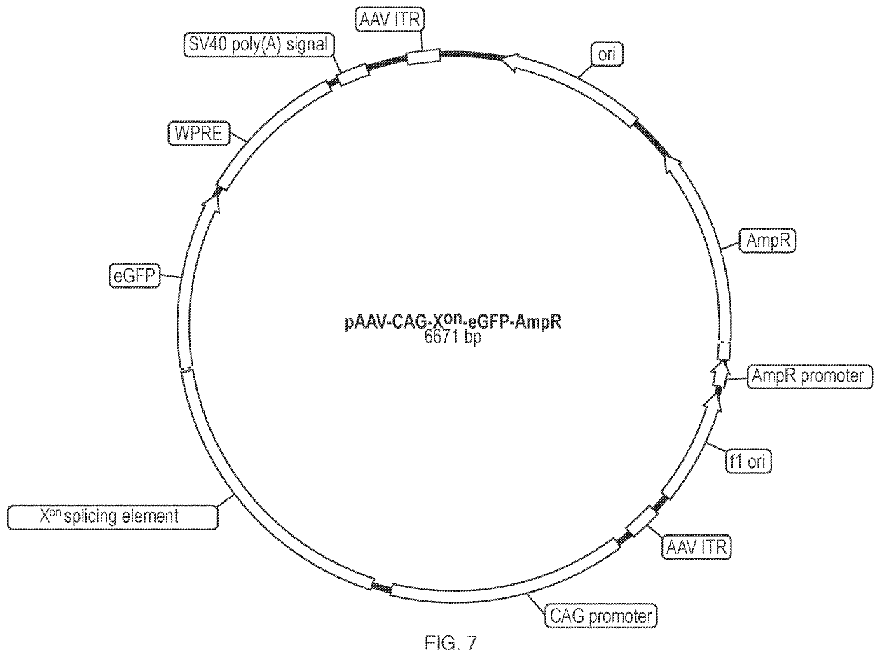

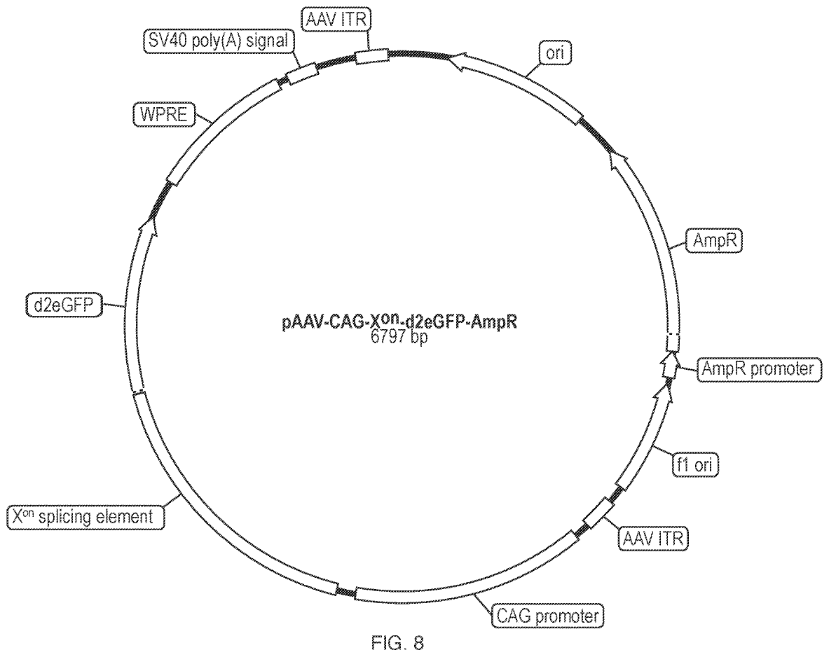

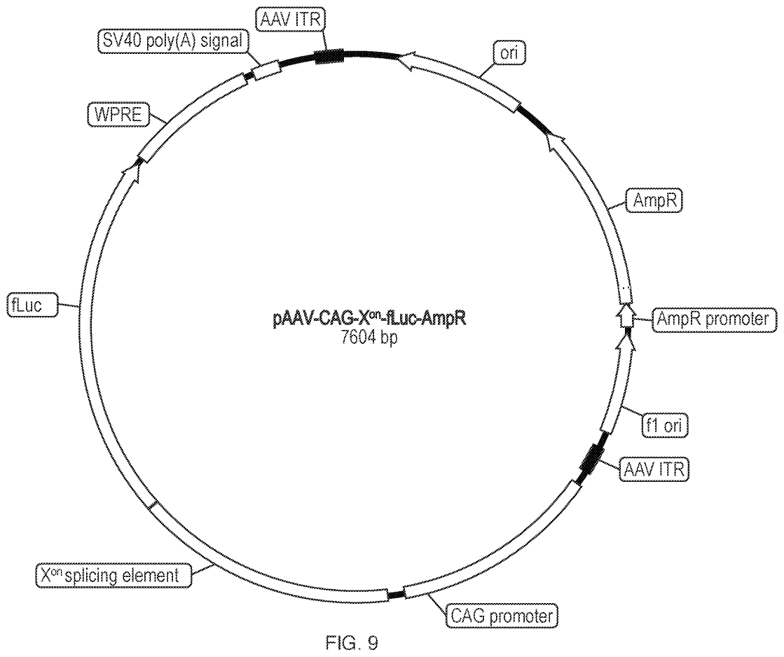

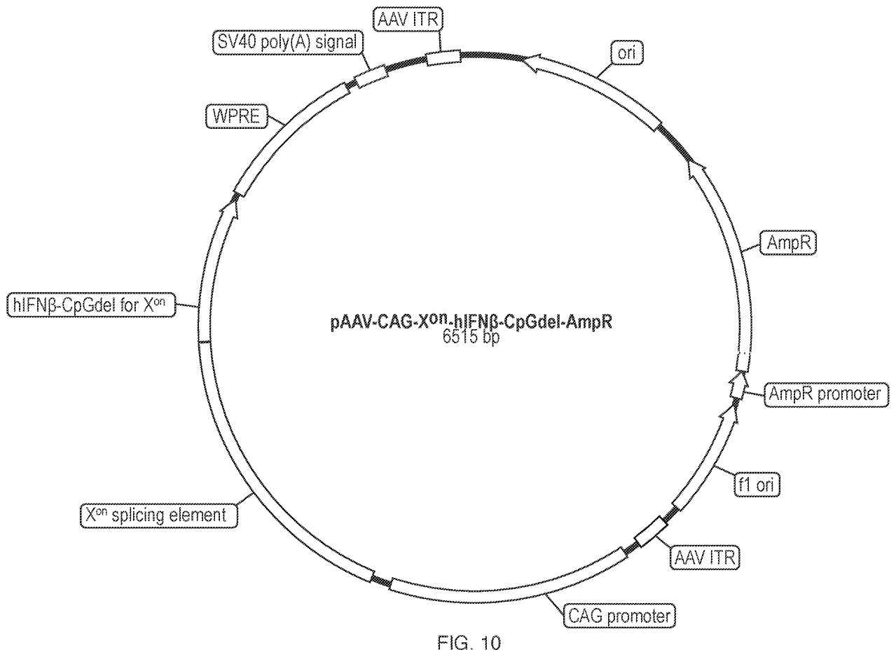

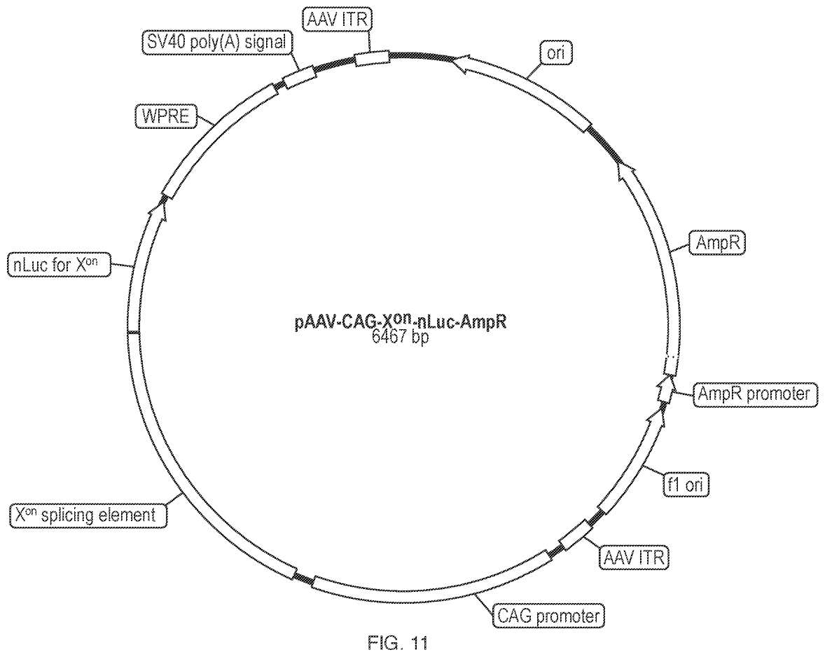

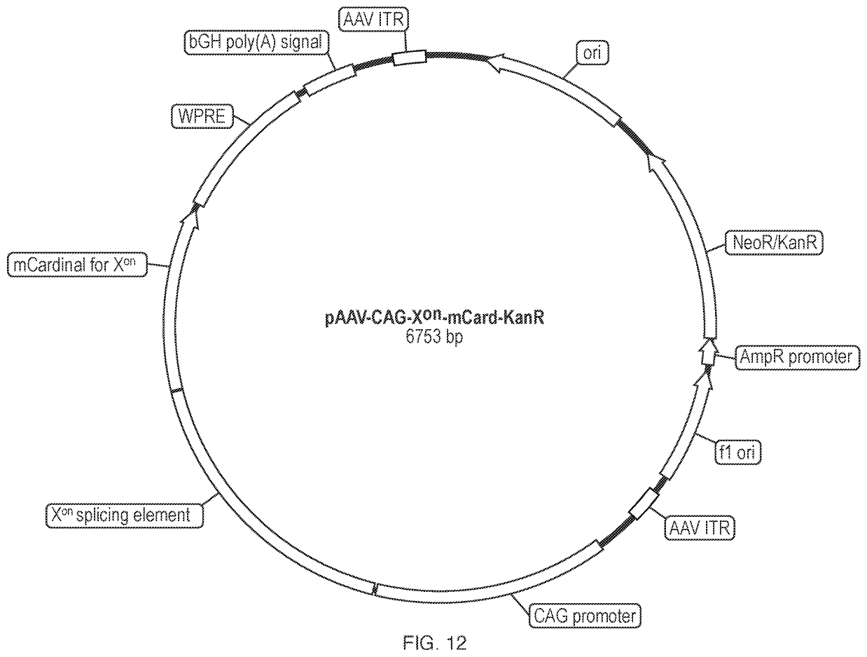

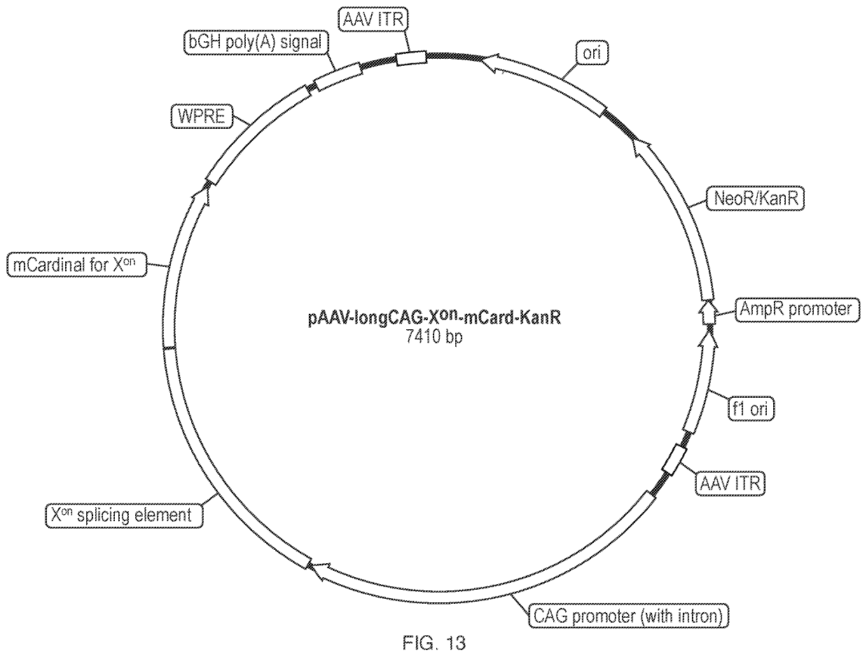

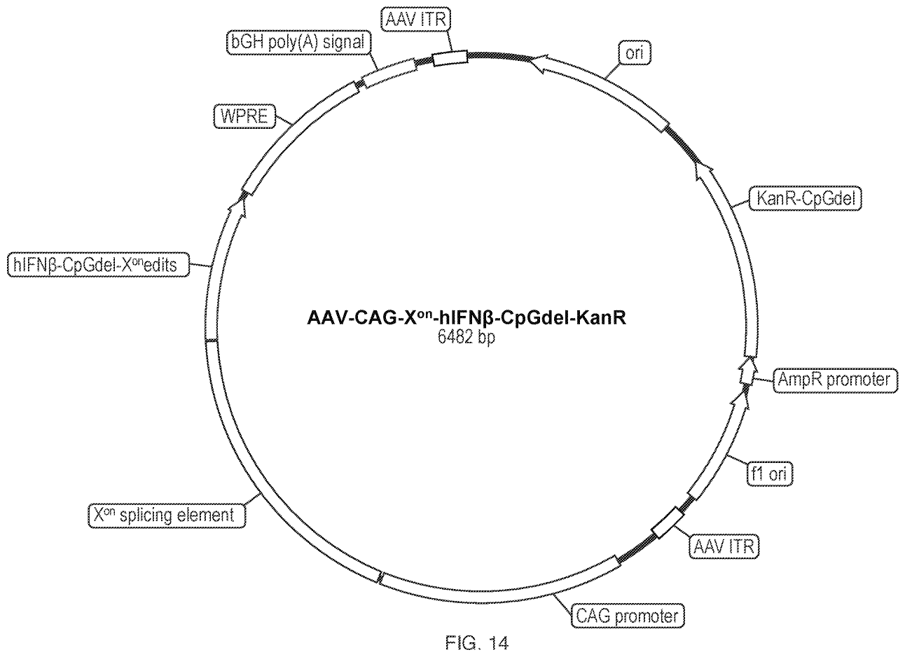

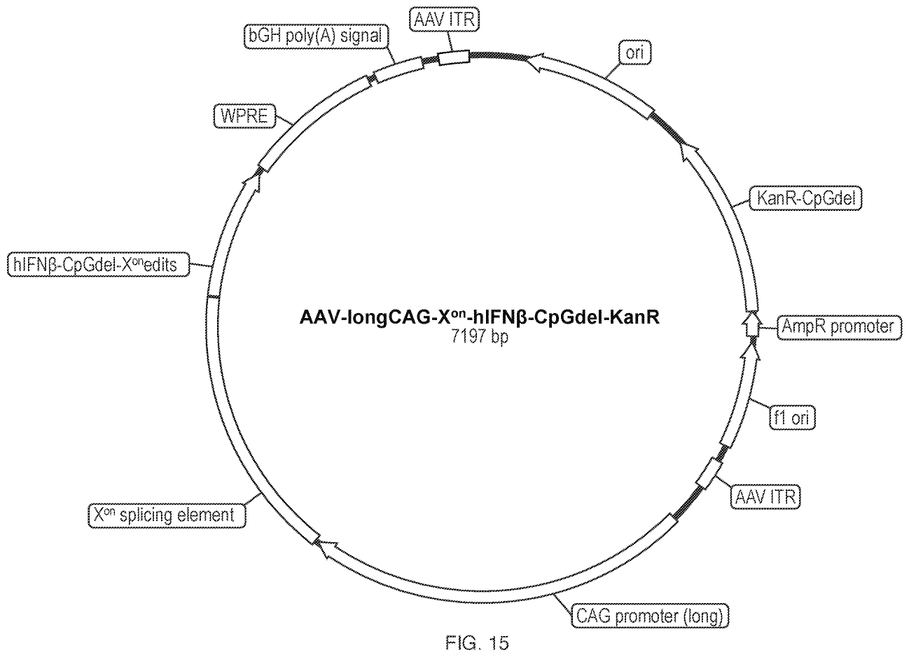

TABLE 1
Plasmids containing AAV genomes with payload
and X^{on} splicing control element

| FIG. Number | Plasmid ID |
|---|---|
| FIG. 2 | pAAV-CAG-X^{on}-eGFP-AmpR |
| FIG. 3 | pAAV-CAG-X^{on}-fLuc-AmpR |
| FIG. 4 | pAAV-CAG-X^{on}-eGFP-KanR |
| FIG. 5 | pAAV-CAG-X^{on}-fLuc-KanR |
| FIG. 6 | pAAV-CAG-X^{on}-hIFNβ-CpGdel-KanR |
| FIG. 7 | pAAV-CAG-X^{on}-eGFP-AmpR |
| FIG. 8 | pAAV-CAG-X^{on}-d2eGFP-AmpR |
| FIG. 9 | pAAV-CAG-X^{on}-fLuc-AmpR |
| FIG. 10 | pAAV-CAG-X^{on}-hIFNβ-CpGdel-AmpR |
| FIG. 11 | pAAV-CAG-X^{on}-nLuc-AmpR |
| FIG. 12 | pAAV-CAG-X^{on}-mCard-KanR |
| FIG. 13 | pAAV-longCAG-X^{on}-mCard-KanR |
| FIG. 14 | pAAV-CAG-X^{on}-hIFNβ-CpGdel-KanR |
| FIG. 15 | pAAV-longCAG-X^{on}-hIFNβ-CpGdel-KanR |

TABLE 2
AAV vector stocks made from the new X^{on}-containing plasmids

| AAV ID | dPCR Titer (vg/mL) |
|---|---|
| AAV-CAG-X^{on}-eGFP-KanR | 2.63E13 |
| AAV-CAG-X^{on}-fLuc-KanR | 1.45E13 |
| AAV-CAG-X^{on}-hIFNβ-CpGdel-KanR | 2.37E13 |
| AAV-CAG-X^{on}-fLuc-AmpR | 3.04E13 |
| AAV-CAG-X^{on}-eGFP-KanR | 3.15E13 |
| AAV-CAG-X^{on}-eGFP-AmpR | 1.1E13 |
| AAV-CAG-X^{on}-nLuc-AmpR | 3.92E13 |

### Example 2. HEK293-T Expression of Inducible AAV-GFP and Inducible AAV-eGFP

This Example describes the validation of inducible payload expression from nucleic acids of the disclosure including a splice modulator binding element.

Adherent human HEK293-T cells were transduced with inducible AAV-GFP, followed by fluorescent imaging for GFP expression pre- and post-administration of an inducer molecule (e.g., branaplam). The data is seen in FIG. 17.

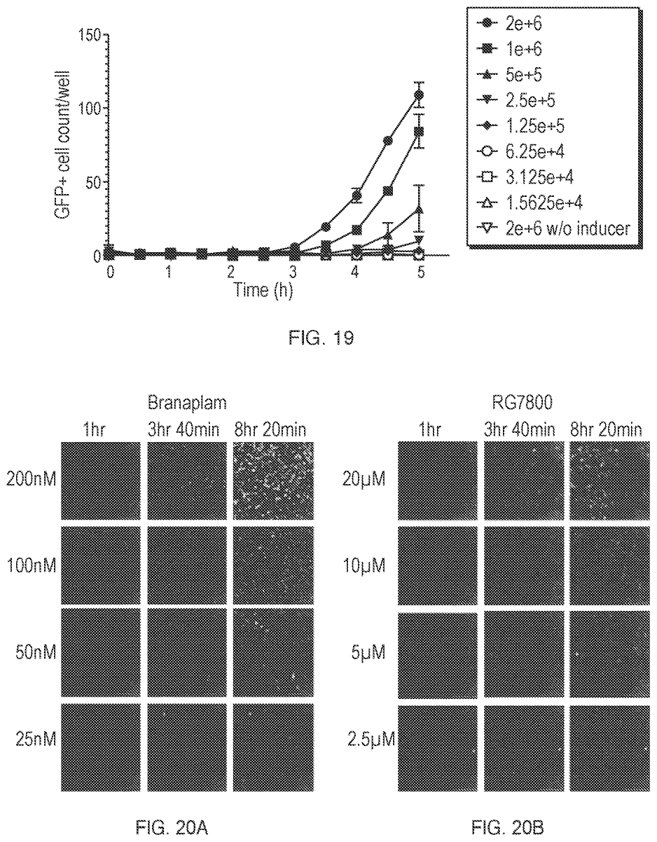

Adherent human HEK293-T cells were transduced with AAV-GFP at varying dosages, followed by administration of an inducer molecule (e.g., branaplam), and monitored for GFP expression over several hours to determine initial expression kinetics at different AAV dosages in vitro. The data is seen in FIG. 19.

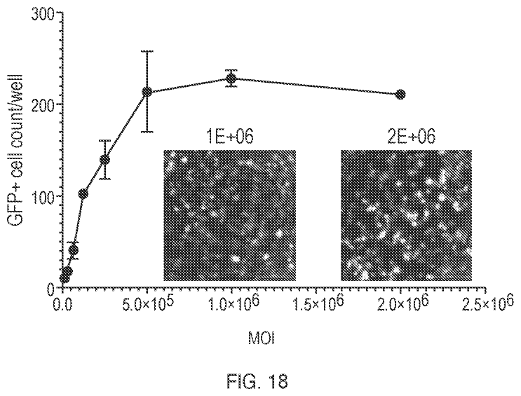

In a similar set of experiments, HEK293-T cells were plated in a 96-well plate for 24 hours and transduced with a broad range of multiplicity of infection (MOI) using a constitutively-expressing payload expression construct, AAV-CAG-eGFP, to determine the necessary MOI for detectable eGFP expression prior to advancing to the inducible constructs described in Example 1. MOIs tested ranged from 3E4 to 4E6 vg/cell. Transduction efficacy was assessed by measuring the eGFP payload expression from transduced cells using a fluorescent microscopy imager (FIG. 18). Once an MOI was selected, the inducible AAV-CAG-X^{on}-eGFP was tested with an inducer molecule, branaplam at 125 nM, 24 hours post-transduction. FIG. 19 shows the eGFP-positive cell count per well obtained by live imaging every 30 minutes and up to 5 hours post-induction. Control cells were transduced with AAV-CAG-X^{on}-eGFP without the addition of an inducer. Branaplam was prepared in DMSO at 10 mM and diluted in the cell media prior to addition to the cell supernatant.

Taken together, the studies showed that MOIs≥1E6 were optimal for transduction of HEK293-T cells using either the constitutive or inducible eGFP constructs described in Example 1. Furthermore, the functionality of the X^{on} splicing element with induction of GFP or eGFP expression, respectively, after addition of the inducer branaplam (also known as LMI070) into the cell media was also demonstrated. These experiments showed the kinetics of induction with detectable GFP and eGFP expression as early as 3 hours post-induction, and with GFP and eGFP signal continuing to increase up to 5 hours post-induction.

Example 3. Dose Studies with Various Inducer Molecules

This Example describes the induction of AAV-eGFP payload expression by four different inducer molecules branaplam version 1, branaplam version 2 from an alternate vendor, RG7800, and RG7800-HCl.

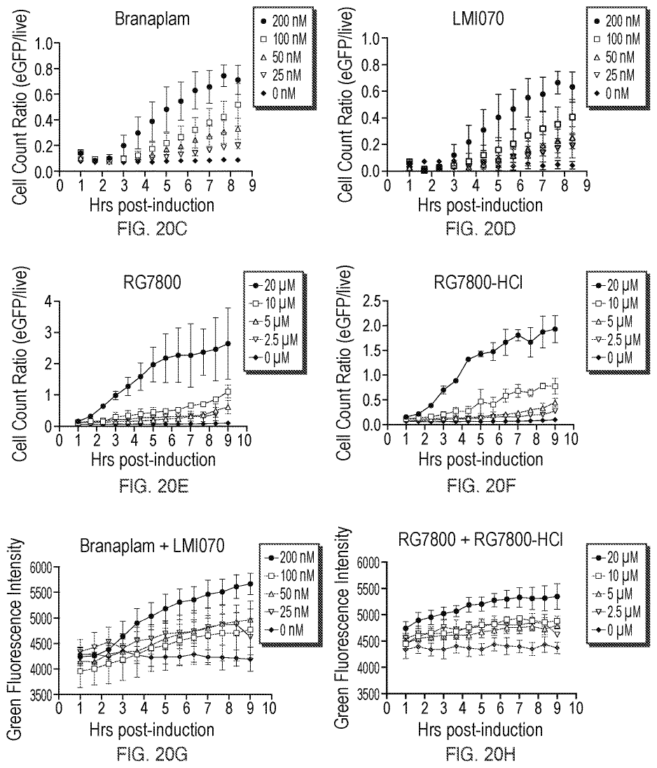

Briefly, cells were seeded in black 96-well plates for 24 hours and transduced with 1e⁶ vg/cell AAV-CAG-X^{on}-eGFP as described in Example 1. After 24 hours, each inducer was added directly into the cell media at 4 different concentrations each. Cells expressing the eGFP payload were visualized by fluorescent live imaging (20× magnification) after induction with branaplam (FIG. 20A) or RG7800 (FIG. 20B). Cells were counter-stained with 20 mM Hoechst to identify nuclei and determine total cell counts for normalization. The number of eGFP-positive cells as well as the green fluorescence intensity was measured for each well and each condition up to 9 hours post-induction to determine the kinetics of the payload expression post-induction (FIGS. 20C-20H).

These experiments demonstrated the functionality of the X^{on} induction system using various small molecule inducers. Further, it was identified that branaplam (also known as LMI070) worked similarly and at the same active concentration of 100 nM to 200 nM, while RG7800 and RG7800-HCl worked similarly and at the same active concentration of 20 μM. This experiment further confirmed that payload expression was detected as early as 3 to 4 hours post-induction and reached a plateau around 9 to 10 hours at the highest dose of each small molecule inducer. Both sets of similarly-performing inducers had a dose-dependent increase in responsiveness.

Example 4. Kinetics of Induction with Branaplam and RG7800-HCl

This Example describes the kinetics of expression in response to the various small molecule inducers.

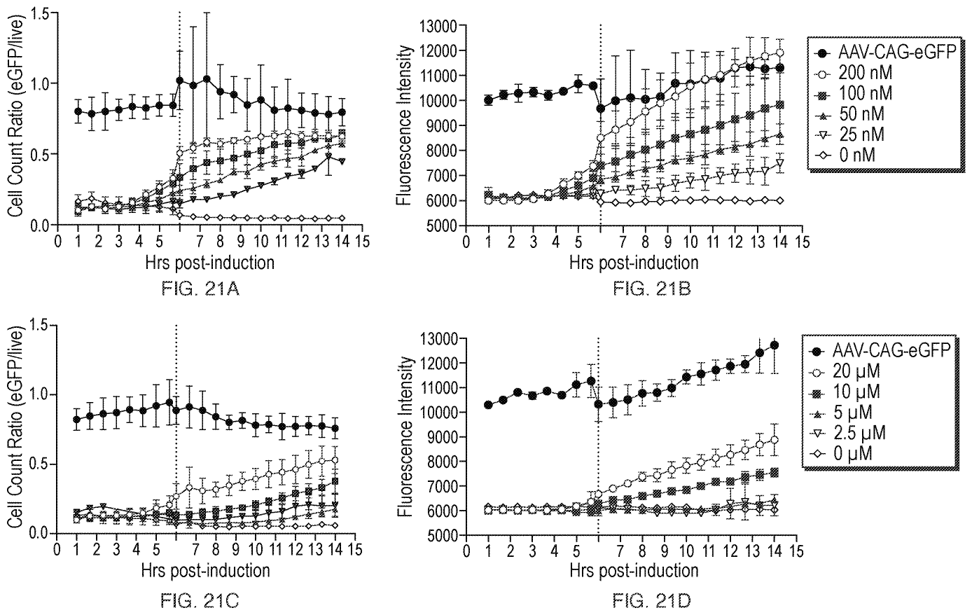

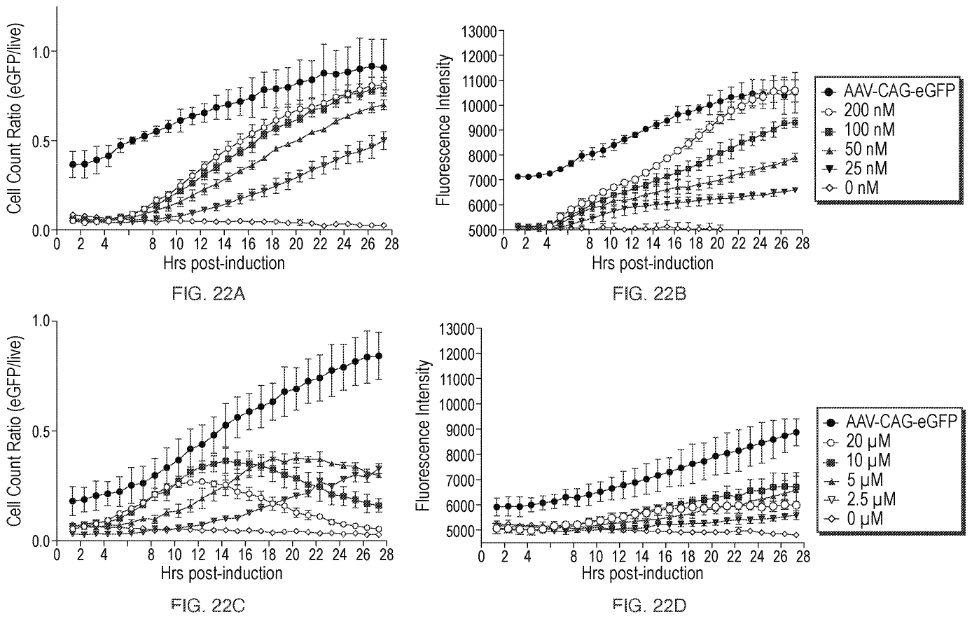

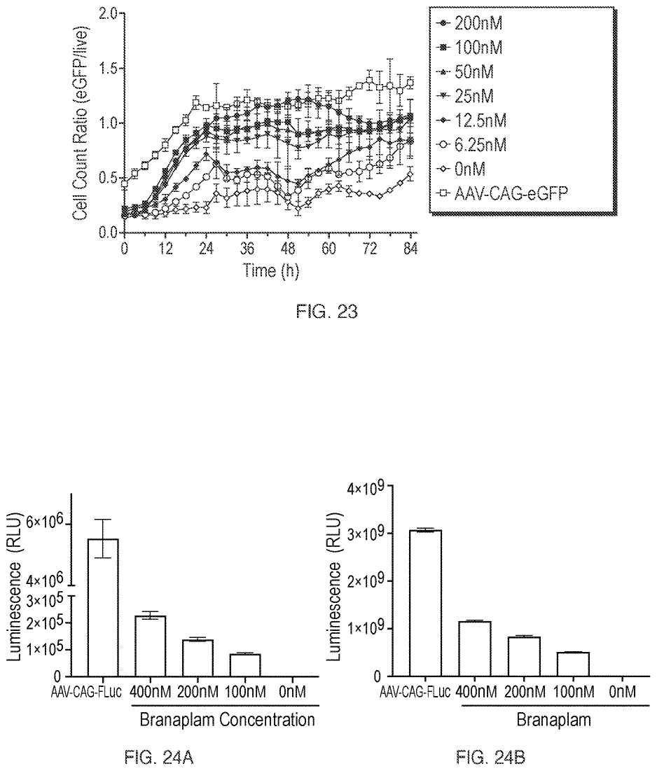

Cells were seeded in 96-well plates for 24 hours and transduced with AAV-CAG-X^{on}-eGFP or constitutive AAV-CAG-eGFP as the positive control, and treated with or without branaplam or RG7800-HCl. Fluorescent eGFP payload expression was monitored for a total of 14 hours (FIGS. 21A-21D) or 28 hours (FIGS. 22A-22D) by live cell imaging. In the experiment with branaplam, eGFP induction was measured a total of 84 hours, as shown in FIG. 23.

Taken together, these experiments showed that the induction of eGFP payload expression led to sustained expression that continued to increase over the first 12 hours, followed by a slower increase to 24 hours. Induced expression levels achieved nearly the same levels of expression as were obtained with the constitutive construct, both with branaplam and RG7800-HCl at optimal concentrations. Because eGFP has a very long half-life, alternative payloads were assessed to identify expression plateaus and alternative kinetics (see Example 5).

Example 5. Induction with Branaplam Using Secreted, High-Sensitivity Luminescent Payloads

This Example describes expression induction using secreted and highly-sensitive luminescent payloads with very short half-lives.

Briefly, HEK293-T cells were transduced at a MOI of 5E5 vg/cell with AAVs including either the firefly Luciferase (fLuc) (FIG. 24A) or the NanoLuc Luciferase (nLuc) (FIG. 24B) genes along with the X^{on} splicing control element. Payload detection was determined by measuring the luminescence on a luminometer 24 hours after induction with branaplam.

Taken together, the results demonstrated that the X^{on} system successfully induced expression of two different secreted luminescent payloads, fLuc and nLuc. Using the AAV-X^{on} constructs, and the matched constitutive versions, approximately 10× lower MOIs were effective to be within the assay detection range than were effective in the eGFP experiments, described in Examples 2-4, given the higher sensitivity obtained with the luciferase expression systems. For these luciferase payloads, the X^{on}-controlled payload expression resulted in lower expression levels than with their respective constitutive version, though still within a highly detectable range.

Example 6. Control of Expression with Branaplam Using Secreted, High-Sensitivity Luminescent Payloads

This Example describes control of payload induction (i.e., turned on and off over time).

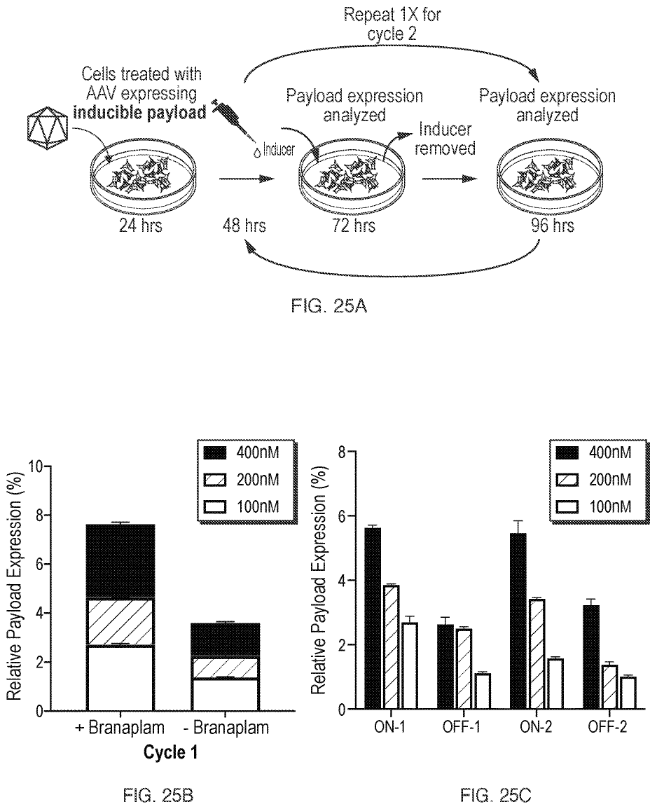

HEK293-T cells were transduced with AAV and then branaplam, and after 24 hours of induction with branaplam, supernatants were harvested and assayed for luciferase expression, and the media was replaced with fresh medium without branaplam. Supernatants for the next timepoint were then harvested after 24 hours in the condition without branaplam, and the cycle was repeated (e.g., one or two or more times; FIG. 25A). Briefly, HEK293-T cells were transduced with an MOI of 2.5E5 vg/cell of AAV-CAG-X^{on}-fLuc (FIG. 25B) for 24 hours, followed by addition of branaplam at 3 different concentrations at hour 24 for an additional 24 hours (“ON”). Supernatants were then harvested for the measurement of fLuc, and media was replaced with fresh media lacking branaplam (“OFF”) for 24 hours. Supernatants were harvested after 24 hours in the OFF state. fLuc luminescent signal was measured for each cycle in sample supernatants at each time point and condition described (FIG. 25C).

Taken together, the results demonstrated that the X^{on} system successfully induced controllable (i.e., on or off) expression of a secreted luminescent payload. The OFF conditions (without branaplam for 24 hours) reliably reduced expression of the fLuc, and re-induction of fLuc expression was achievable and detectable through at least 2 full induction cycles. The higher doses of branaplam (≥200 nM) were needed to mediate reliable payload expression and cycle control.

Example 7. Control of Expression with Branaplam Using a Therapeutic Payload

This Example describes control of therapeutic payload induction (i.e., turned on and off over time).

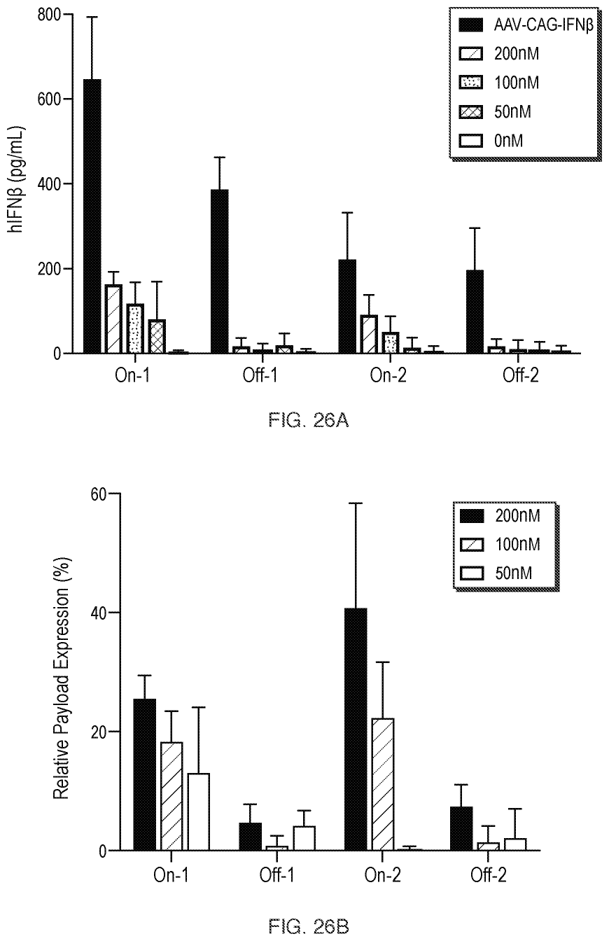

Briefly, HEK293-T cells were transduced with an MOI of 2.5e⁵ vg/cell of AAV-CAG-X^{on}-hIFNβ or constitutive AAV-CAG-hIFNβ for 24 hours. 24 hours post-transduction with AAV, branaplam was added to the cell media to induce human IFNβ (hIFNβ) expression (“ON”). After 24 hours, media supernatants were harvested for payload measurement and fresh media lacking Branaplam (“OFF”). 24 hours later, media supernatants were harvested for payload detection (FIG. 25A). Amounts of hIFNβ were measured in picograms per milliliter (pg/mL) (FIG. 26A) using an ELISA kit for hIFNβ in supernatants before and after induction with branaplam, and after removal of branaplam. FIG. 26B depicts the expression of hIFNβ following transduction with AAV-CAG-X^{on}-hIFNβ normalized to the levels of expression obtained with the constitutive payload.

Taken together, the results demonstrated that branaplam was able to successfully repeat induced expression of a therapeutic payload e.g., hIFNβ within 24 hours of induction. Removal of branaplam successfully stopped hIFNβ expression within 24 hours of removal for up to 2 repeat cycles on the same transduced cells.

### Example 8. Control of Expression In Vivo

Experiment 1: wild-type (WT) non-tumor bearing mice are used to demonstrate feasibility of the X^{on} splicing control element in vivo. To validate payload expression and lack of AAV-CAG-X^{on}-IFNβ toxicity, first, the tolerability of an inducer (e.g., branaplam, LMI070, RG7916, RG7800, RG7800-HCl, or sudemycin) is tested in WT mice in a 5-arm study. In an experiment, branaplam is tested at four doses (5 mg/kg, 25 mg/kg, 50 mg/kg, and 75 mg/kg) against a dimethyl sulfoxide (DMSO) vehicle (Table 3) control arm. Mice are administered branaplam daily by oral gavage for up to 7 days to establish safety or acute toxicity of branaplam (without AAV). Mortality/morbidity are assessed 2 times per day. Body weight is measured on Days −1, 1, 3 and 7. At study termination, animals undergo macroscopic examination, organ weight assessment, hematology, clinical chemistry, and urinalysis.

TABLE 3

| Study Arms in WT Mice | Treatment | Harvest |
|---|---|---|
| Branaplam in DMSO | Oral branaplam 5, 25, 50, and 75 | Day 7 |
| | mg/kg | |
| Vehicle control | DMSO vehicle (same volume, 15 μL) | Day 7 |

Experiment 2: to establish an effective dose range for single dose oral branaplam in WT mice, the optimal 2E11 vg/mouse AAV dose from prior experiments are used and experiments are conducted with the inducible AAV-CAG-X^{on}-fLuc. Mice are transduced with inducible AAV-CAG-X^{on}-fLuc and administered daily oral branaplam 2 days after receiving AAV, up to 7 days. This establishes the minimum and maximum oral doses of branaplam needed to induce fLuc expression. Two control arms are provided constitutive AAV-CAG-fLuc or AAV-CAG-X^{on}-fLuc without branaplam (Table 4). fLuc expression is quantified with daily bioluminescent imaging, and tissues are harvested on Day 7 for analyses, such as immunohistochemistry and RNA sequencing (RNA-seq) to identify splice variants.

TABLE 4

| Study Arms in WT Mice | Treatment | Harvest |
|---|---|---|
| AAV-CAG-X^{on}-fLuc + | Inducible AAV 2E11 + | Day 7 |
| branaplam | branaplam dose 1 | |
| AAV-CAG-X^{on}-fLuc + | Inducible AAV 2E11 + | Day 7 |
| branaplam | branaplam dose 2 | |
| AAV-CAG-X^{on}-fLuc negative | Inducible AAV 2E11 − | Day 7 |
| control | branaplam | |
| AAV-CAG-fLuc positive control | Constitutive AAV 2E11 | Day 7 |

Experiment 3: to demonstrate the ability of branaplam to turn payload expression on and off repeatedly in WT mice, additional experiments are conducted. After establishing branaplam dosing from Experiment 2, described above, branaplam administration cycles are tested in WT mice e.g., for two on/off cycles by providing daily oral branaplam 2 days after AAV administration up to 7 days. For the ‘off’ condition, branaplam is not provided until expression returns to baseline (e.g., for about 24 hours). To begin the next ‘on’ cycle, branaplam is re-administered daily orally until day 14. Two control arms are given constitutive AAV-CAG-fLuc or AAV-CAG-X^{on}-fLuc minus branaplam (Table 5). fLuc expression is quantified with daily bioluminescent imaging. Tissues are harvested on Day 14 for analyses, including immunohistochemistry and RNA-seq for splice variant analysis.

TABLE 5

| Study Arms in WT Mice | Treatment | Harvest |
|---|---|---|
| AAV-CAG-X^{on}-fLuc + | Inducible AAV 2E11 + | Day 14 |
| branaplam 2 cycles | branaplam on/off/on 14 days | |
| AAV-CAG-X^{on}-fLuc negative | Inducible AAV 2E11 − | Day 14 |
| control | branaplam | |
| AAV-CAG-fLuc positive control | Constitutive AAV 2E11 | Day 14 |

Experiment 4: to validate inducible IFNβ cytokine expression in WT mice additional experiments are conducted. Using inducible AAV-CAG-X^{on}-IFNβ (FIGS. 14 and 15), IFNβ expression is validated in WT mice following administration to the brain parenchyma by convection-enhanced delivery (CED). Daily oral branaplam is administered 2 days after AAV administration out to 7 days. Two control arms are given constitutive AAV-CAG-IFNβ or AAV-CAG-X^{on}-IFNβ without branaplam (Table 6). Tissues are harvested on Day 7 for IFNβ immunohistochemistry and RNA-seq for splice variant analysis. This study validates expression at both the protein and transcript levels and confirms fidelity of induction in vivo with a clinically compatible IFNβ payload.

TABLE 6

| Study Arms in WT Mice | Treatment | Harvest |
|---|---|---|
| AAV-CAG-X^{on}-IFNβ + | Inducible AAV 2E11 + | Day 7 |
| branaplam 1 cycle | branaplam on 5 days | |
| AAV-CAG-X^{on}-IFNβ | Inducible AAV 2E11 − | Day 7 |
| negative control | branaplam | |
| AAV-CAG-IFNβ positive control | Constitutive AAV 2E11 | Day 7 |

Experiment 5: to validate the inducible system in vivo in tumor-bearing mice, similar experiments as Experiment 4, described above, are performed in fLuc-positive tumor-bearing xenograft mice and the functional expression of IFNβ is assessed. In a 4-arm study, short-term tumor growth is assessed via bioluminescent imaging of fLuc-positive brain tumors. This study establishes that functional IFNβ expression is observed in a tumor-bearing animal model.

To establish orthotopic tumors of an exemplary cancer, such as glioblastoma (GBM), 5 to 6-week old female athymic mice are injected intraparenchymally with about 300,000 human fLuc-positive GBM cells. Once symptomatic (after approximately 7 to 10 days), xenograft mice are randomized to a treatment group based on tumor size (assessed via bioluminescent imaging of fLuc-positive tumors) and AAV is administered intratumorally using CED. Daily oral branaplam is administered at a dose based upon Experiment 2, described above. Dosing of branaplam is conducted 2 days after AAV administration, out to 14 days, and tissue is harvested from mice at Day 30. Control mice are administered vehicle, constitutive AAV-CAG-IFNβ, or AAV-CAG-X^{on}-IFNβ without branaplam (Table 7). Measurable outcomes include weekly tumor imaging, overall survival, and histopathology on FFPE brain tissues (H&E and hIFNβ) at study end. Brains are also retained for RNA-seq splice variant analysis.

TABLE 7

| Study Arms in Tumor-Bearing | | |
|---|---|---|
| Xenograft Mice | Treatment | Harvest |
| AAV-CAG-X^{on}-IFNβ + | Inducible AAV 2E11 + | Day 30 |
| branaplam 1 cycle | branaplam on 12 days | |
| AAV-CAG-X^{on}-IFNβ | Inducible AAV 2E11 − | Once |
| negative control | branaplam | symptomatic |
| AAV-CAG-IFNβ positive | Constitutive AAV 2E11 | Day 30 |
| control | | |
| Vehicle negative control | Saline (same volume, 15 | Once |
| | μL) | symptomatic |

Experiment 6: to evaluate the efficacy of AAV-CAG-X^{on}-IFNβ in a high-powered preclinical study in tumor-bearing animals, additional studies are conducted. Five to six week old female athymic mice are injected intraparenchymally with about 300,000 human fLuc-positive GBM cells. Once symptomatic (after approximately 7-10 days), xenograft mice are randomized into treatment groups based on tumor size (assessed via bioluminescent imaging of fLuc-positive tumors) and AAV is administered intratumorally using CED. Branaplam is administered daily orally at a dose based upon Experiment 2, described above, two days after AAV administration, out to 14 days. Tissue is harvested from mice 40 days post-AAV. Three control arms are administered vehicle, constitutive AAV-CAG-IFNβ, or AAV-CAG-X^{on}-IFNβ minus branaplam (Table 8).

TABLE 8

| Study Arms in GBM Xenograft | | |
|---|---|---|
| Mice | Treatment | Harvest |
| AAV-CAG-X^{on}-IFNβ + | Inducible AAV 2E11 + | Day 42 |
| branaplam 1 cycle | branaplam on 12 days | |
| AAV-CAG-X^{on}-IFNβ | Inducible AAV 2E11 − | Once |
| negative control | branaplam | symptomatic |
| AAV-CAG-IFNβ positive | Constitutive AAV 2E11 | Day 42 |
| control | | |
| Vehicle control | Saline (same volume, 15 | Once |
| | μL) | symptomatic |

Weekly tumor imaging, overall survival, and histopathology on formalin-fixed paraffin-embedded (FFPE) brain tissues (H&E and hIFNβ) are conducted at the end of the study. Brain tissues are also examined with RNA-seq for splice variant analysis.

1. A viral vector comprising a nucleic acid comprising a first polynucleotide comprising a minigene 5′ of a second polynucleotide encoding an interferon, wherein the minigene comprises a splice modulator binding site.

2. The viral vector of claim 1, further comprising a promoter, an enhancer, an intron, a microRNA, a linker, a splicing element, a regulatory element, or a polyA signal.

3. The viral vector of claim 1, further comprising a promoter.

4. The viral vector of claim 2 or 3, wherein the promoter is selected from the group consisting of: a mini promoter, an inducible promoter, a constitutive promoter, and derivatives thereof.

5. The viral vector of claim 2 or 3, wherein the promoter is selected from the group consisting of: CMV, CBA, EF1a, CAG, PGK, TRE, U6, UAS, T7, Sp6, lac, araBad, trp, Ptac, p5, p19, p40, Synapsin, CaMKII, GRK1, and derivatives thereof.

6. The viral vector of any one of claims 1-5, wherein the first polynucleotide and the second polynucleotide are linked by a cleavable peptide.

7. The viral vector of claim 6, wherein the cleavable peptide is a self-cleaving peptide, a drug-sensitive protease, or a substrate for an endogenous endoprotease.

8. The viral vector of any one of claims 1-7, wherein the minigene is regulated by a small molecule splicing modifier.

9. The viral vector of claim 8, wherein the small molecule splicing modifier is sudemycin, LMI070, RG7916, RG7800, or a salt derivative thereof.

10. The viral vector of claim 9, wherein the salt derivative thereof is RG7800-HCl.

11. The viral vector of claim 8, wherein the small molecule splicing modifier is selected from the group consisting of:

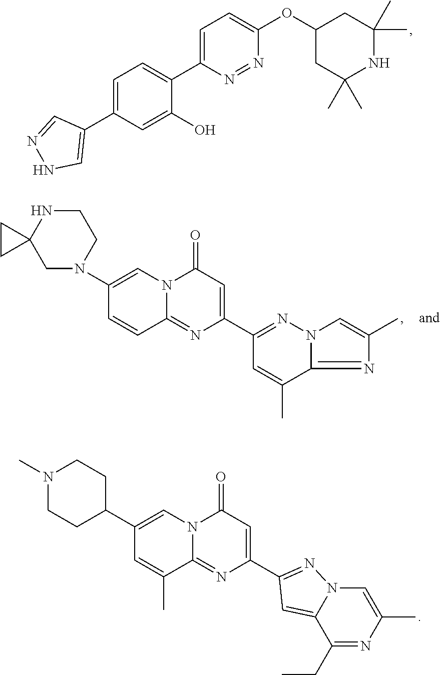

12. The viral vector of any one of claims 1-11, wherein the minigene comprises exons 6, 7, and 8 of SMN2 and the splice modulator binding site is recognized by LMI070.

13. The viral vector of any one of claims 1-11, wherein the minigene comprises at least about 80% sequence identity to the nucleic acid sequence of SEQ ID NO: 32 or SEQ ID NO: 33.

14. The viral vector of any one of claims 1-11, wherein the minigene is regulated by a disease state in a cell.

15. The viral vector of claim 14, wherein the disease state is cancer.

16. The viral vector of claim 15, wherein the cancer is glioblastoma.

17. The viral vector of any one of claims 1-16, wherein the minigene is regulated by a cell type or tissue type.

18. The viral vector of any one of claims 1-17, wherein the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus.

19. The viral vector of claim 18, wherein the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2YF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof.

20. The viral vector of claim 18, wherein the herpesvirus is HSV-1, HSV-2, VZV, EBV, CMV, HHV-6, HHV-7, or HHV-8.

21. The viral vector of any one of claims 1-20, wherein the interferon (IFN) is IFNα, IFNβ, IFNγ, IFNε, IFNκ, IFNω, IFNλ, derivatives thereof, or combinations thereof.

22. The viral vector of any one of claims 1-21, further comprising a third polynucleotide comprising a minigene 5′ of a fourth polynucleotide encoding an interferon.

23. The viral vector of any one of claims 1-22, further comprising a fifth polynucleotide comprising a minigene 5′ of a sixth polynucleotide encoding an interferon.

24. The viral vector of claim 22 or 23, wherein the interferons are the same.

25. The viral vector of claim 22 or 23, wherein the interferons are different.

26. The viral vector of any one of claims 1-25, further comprising a backbone polynucleotide.

27. The viral vector of claim 26, wherein the backbone polynucleotide comprises an antibiotic resistance gene.

28. The viral vector of claim 27, wherein the antibiotic resistance gene encodes an aminoglycoside, a beta-lactam, a macrolide, a tetracycline, or a derivative thereof.

29. The viral vector of claim 27, wherein the antibiotic resistance gene encodes kanamycin, spectinomycin, streptomycin, ampicillin, carbenicillin, bleomycin, erythromycin, polymyxin B, tetracycline, chloramphenicol, neomycin, zeocin, or a derivative thereof.

30. The viral vector of claim 29, wherein the antibiotic resistance gene encodes a kanamycin resistance gene.

31. The viral vector of claim 29, wherein the kanamycin resistance gene comprises a nucleic acid sequence having at least about 80% sequence identity to the nucleic acid sequence of any one of SEQ ID NOs: 20 or 21.

32. The viral vector of claim 29, wherein the antibiotic resistance gene encodes an ampicillin resistance gene.

33. The viral vector of claim 32, wherein the ampicillin resistance gene comprises a nucleic acid sequence having at least about 80% sequence identity to the nucleic acid sequence of SEQ ID NO: 34.

34. The viral vector of any one of claims 26-33, wherein the backbone polynucleotide comprises an origin of replication.

35. The viral vector of claim 34, wherein the origin of replication is selected from the group consisting of: pMB1, pBR322, ColE1, R6K, p15A, pSC101, ColE2, F1, pUC, and combinations or a derivative thereof.

36. The viral vector of any one of claims 1-35, wherein the viral vector comprises the nucleic acid sequence of SEQ ID NO: 31.

37. A viral vector, comprising from 5′ to 3′:
(a) a 5′ AAV inverted terminal repeat (ITR);
(b) a CAG promoter;
(c) a first polynucleotide comprising a minigene, the minigene comprising a splice modulator binding site;
(d) a second polynucleotide encoding an interferon; and
(e) a 3′ AAV ITR.

38. The viral vector of claim 37, wherein the CAG promoter comprises a nucleic acid sequence having at least 80% sequence identity to the nucleic acid sequence of any one of SEQ ID NOs: 22, 23, 44, or 45.

39. The viral vector of any one of claims 37-38, wherein the minigene comprises at least about 80% sequence identity to the nucleic acid sequence of SEQ ID NO: 32 or SEQ ID NO: 33.

40. The viral vector of any one of claims 37-39, wherein the interferon is encoded by a nucleic acid sequence having at least 80% sequence identity to the nucleic acid sequence of any one of SEQ ID NOs: 2-4, 6-8, 10-12, and 46.

41. A viral vector, comprising from 5′ to 3′:
(a) a 5′ AAV inverted terminal repeat (ITR);
(b) a CAG promoter comprising a nucleic acid sequence having at least 80% sequence identity to the nucleic acid sequence of any one of SEQ ID NOs: 22, 23, 44, or 45;
(c) a first polynucleotide comprising a minigene, the minigene comprising a splice modulator binding site and at least about 80% sequence identity to the nucleic acid sequence of SEQ ID NO: 32 or SEQ ID NO: 33;
(d) a second polynucleotide encoding an interferon, the second polynucleotide comprising at least about 80% sequence identity to the nucleic acid sequence of SEQ ID NO: 4; and
(e) a 3′ AAV ITR.

42. The viral vector of any one of claims 37-41, wherein the minigene is regulated by a small molecule splicing modifier.

43. The viral vector of claim 42, wherein the small molecule splicing modifier is sudemycin, LMI070, RG7916, RG7800, or a salt derivative thereof.

44. The viral vector of claim 43, wherein the salt derivative thereof is RG7800-HCl.

45. The viral vector of claim 42, wherein the small molecule splicing modifier is selected from the group consisting of:

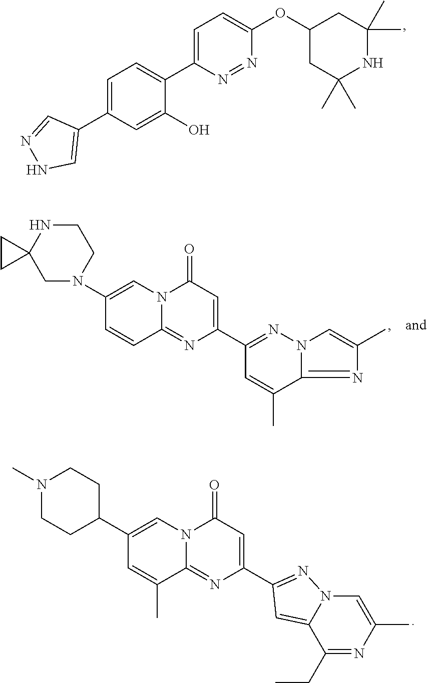

46. The viral vector of any one of claims 37-45, wherein the viral vector further comprises an antibiotic resistance gene sequence.

47. The viral vector of claim 46, wherein the antibiotic resistance gene is a kanamycin resistance gene.

48. The viral vector of claim 47, wherein the kanamycin resistance gene comprises a nucleic acid sequence having at least 80% sequence identity to the nucleic acid sequence of any one of SEQ ID NOs: 20 or 21.

49. The viral vector of claim 46, wherein the antibiotic resistance gene is an ampicillin resistance gene.

50. The viral vector of claim 49, wherein the ampicillin resistance gene comprises a nucleic acid sequence having at least 80% sequence identity to the nucleic acid sequence of any one of SEQ ID NOs: 20 or 21.

51. A method of treating cancer in a subject in need thereof comprising: administering a viral vector of any one of claims 1-50.

52. The method of claim 51, wherein the subject is a human.

53. The method of claim 51, wherein the subject is a mouse, rat, equine, sheep, cow, primate, dog, cat, pig, donkey, or rabbit.

54. The method of claim 51-53, wherein the administration is to the central nervous system.

55. The method of any one of claims 51-54, wherein the administration is to a brain.

56. The method of any one of claims 51-55, wherein the administration is to a brain ventricle.

57. The method of any one of claims 51-56, wherein the administration is by convection enhanced delivery (CED).

58. The method of any one of claims 51-56, wherein the administration is by intratumoral injection, intracranial injection, intracerebral injection, intracerebroventricular, or injection into the cerebrospinal fluid (CSF) via the cerebral ventricular system, cisterna magna, or intrathecal space.
